SEQUENCE LISTING (30 sequences)

SEQ ID NO: 1 - protein, 17 aa, Artificial sequence
  sequence is synthesized
KSSQSLLYTSSQKNYLA

SEQ ID NO: 2 - protein, 7 aa, Artificial sequence
  sequence is synthesized
WASTRES

SEQ ID NO: 3 - protein, 9 aa, Artificial sequence
  sequence is synthesized
QQYYAYPWT

SEQ ID NO: 4 - protein, 10 aa, Artificial sequence
  sequence is synthesized
GYTFTSYWLH

SEQ ID NO: 5 - protein, 18 aa, Artificial sequence
  sequence is synthesized
GMIDPSNSDTRFNPNFKD

SEQ ID NO: 6 - protein, 11 aa, Artificial sequence
  sequence is synthesized
XYGSYVSPLDY

SEQ ID NO: 7 - protein, 11 aa, Artificial sequence
  sequence is synthesized
TYGSYVSPLDY

SEQ ID NO: 8 - protein, 11 aa, Artificial sequence
  sequence is synthesized
SYGSYVSPLDY

SEQ ID NO: 9 - protein, 12 aa, Artificial sequence
  sequence is synthesized
ATYRSYVTPLDY

SEQ ID NO: 10 - protein, 119 aa, Artificial sequence [sequence omitted]

SEQ ID NO: 11 - protein, 114 aa, Artificial sequence
  sequence is synthesized
DIQMTQSPSSLSASVGDRVTITCKSSQSLLYTSSQKNYLAWYQQKPGKAPKLLIYWASTRESGVPSRFSGSGSGTDFTLTISSLQPEDFATYYCQQYYAYPWTFGQGTKVEIKR

SEQ ID NO: 12 - protein, 222 aa, Artificial sequence
  sequence is synthesized
CPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 13 - protein, 222 aa, Artificial sequence
  sequence is synthesized
CPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 14 - protein, 449 aa, Artificial sequence
  sequence is synthesized
EVQLVESGGGLVQPGGSLRLSCAASGYTFTSYWLHWVRQAPGKGLEWVGMIDPSNSDTRFNPNFKDRFTISADTSKNTAYLQMNSLRAEDTAVYYCATYRSYVTPLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 15 - protein, 220 aa, Artificial sequence
  sequence is synthesized
DIQMTQSPSSLSASVGDRVTITCKSSQSLLYTSSQKNYLAWYQQKPGKAPKLLIYWASTRESGVPSRFSGSGSGTDFTLTISSLQPEDFATYYCQQYYAYPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 16 - protein, 23 aa, Artificial sequence
  sequence is synthesized
DIQMTQSPSSLSASVGDRVTITC

SEQ ID NO: 17 - protein, 15 aa, Artificial sequence
  sequence is synthesized
WYQQKPGKAPKLLIY

SEQ ID NO: 18 - protein, 32 aa, Artificial sequence
  sequence is synthesized
GVPSRFSGSGSGTDFTLTISSLQPEDFATYYC

SEQ ID NO: 19 - protein, 11 aa, Artificial sequence
  sequence is synthesized
FGQGTKVEIKR

SEQ ID NO: 20 - protein, 106 aa, Artificial sequence
  sequence is synthesized
TVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 21 - protein, 25 aa, Artificial sequence [sequence omitted]

SEQ ID NO: 22 - protein, 13 aa, Artificial sequence
  sequence is synthesized
WVRQAPGKGLEWV

SEQ ID NO: 23 - protein, 30 aa, Artificial sequence
  sequence is synthesized
RFTISADTSKNTAYLQMNSLRAEDTAVYYC

SEQ ID NO: 24 - protein, 11 aa, Artificial sequence
  sequence is synthesized
WGQGTLVTVSS

SEQ ID NO: 25 - protein, 5 aa, Homo sapiens
LDAQT

SEQ ID NO: 26 - protein, 9 aa, Homo sapiens
LTEKRKKRS

SEQ ID NO: 27 - protein, 8 aa, Homo sapiens
KPDSAEPM

SEQ ID NO: 28 - protein, 8 aa, Homo sapiens
NVRCLQHF

SEQ ID NO: 29 - protein, 11 aa, Artificial sequence
  sequence is synthesized
DICLPRWGCLW

SEQ ID NO: 30 - protein, 108 aa, Homo sapiens
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHT

(19) United States
(12) Patent Application Publication
(10) Pub. No.: US 2011/0262436 A1
(43) Pub. Date: Oct. 27, 2011

(54) TREATMENT METHOD

(75) Inventors: Brendan C. Bender, Uppsala (SE); Steve Eppler, San Mateo, CA (US); Priti Hegde, San Mateo, CA (US); Nelson L. Jumbe, Mountain View, CA (US); Mark Merchant, Belmont, CA (US); Amy C. Peterson, San Francisco, CA (US); Arthur E. Reyes, II, Palo Alto, CA (US); Hong Xiang, Palo Alto, CA (US)

(73) Assignee: Genentech, Inc., San Francisco, CA (US)

(21) Appl. No.: 13/124,486

(22) PCT Filed: Oct. 14, 2009
(86) PCT No.: PCT/US09/60662

§ 371 Date: Jul. 11, 2011

Related U.S. Application Data
(60) Provisional application No. 61/106,495, filed on Oct. 17, 2008.
(60) Provisional application No. 61/152,570, filed on Feb. 13, 2009.

(51) Int. Cl. A61K 39/395; A61P 35/00; C12Q 1/02

(52) U.S. Cl. 424/133.1; 424/138.1; 435/29

(57) ABSTRACT

The present invention relates generally to the fields of molecular biology and growth factor regulation. More specifically, the invention relates to therapies for the treatment of pathological conditions, such as cancer.

### RELATED APPLICATIONS

This application claims priority under 35 USC 119(e) to U.S. provisional patent application No. 61/106,495, filed Oct. 17, 2008, and U.S. provisional patent application No. 61/152,570, filed Feb. 13, 2009, the contents of which are incorporated herein by reference.

### TECHNICAL FIELD

The present invention relates generally to the fields of molecular biology and growth factor regulation. More specifically, the invention relates to combination therapies for the treatment of pathological conditions, such as cancer.

### BACKGROUND

Cancer is one of the most deadly threats to human health. In the U.S. alone, cancer affects nearly 1.3 million new patients each year, and is the second leading cause of death after cardiovascular disease, accounting for approximately 1 in 4 deaths. Solid tumors are responsible for most of those deaths. Although there have been significant advances in the medical treatment of certain cancers, the overall 5-year survival rate for all cancers has improved only by about 10% in the past 20 years. Cancers, or malignant tumors, metastasize and grow rapidly in an uncontrolled manner, making timely detection and treatment extremely difficult.

HGF is a mesenchyme-derived pleiotrophic factor with mitogenic, motogenic and morphogenic activities on a number of different cell types. HGF effects are mediated through a specific tyrosine kinase, c-met, and aberrant HGF and c-met expression are frequently observed in a variety of tumors. See, e.g., Maulik et al., Cytokine & Growth Factor Reviews (2002), 13:41-59; Danilkovitch-Miagkova & Zbar, J. Clin. Invest. (2002), 109(7):863-867. Regulation of the HGF/c-Met signaling pathway is implicated in tumor progression and metastasis. See, e.g., Trusgolino & Comoglio, Nature Rev. (2002), 2:289-300).

HGF binds the extracellular domain of the Met receptor tyrosine kinase (RTK) and regulates diverse biological processes such as cell scattering, proliferation, and survival. HGF-Met signaling is essential for normal embryonic development especially in migration of muscle progenitor cells and development of the liver and nervous system (Bladt et al., Nature (1995), 376, 768-771; Hamanoue et al., Faseb J (2000), 14, 399-406; Maina et al., Cell (1996), 87, 531-542; Schmidt et al., Nature (1995), 373, 699-702; Uehara et al., Nature (1995), 373, 702-705). Developmental phenotypes of Met and HGF knockout mice are very similar suggesting that HGF is the cognate ligand for the Met receptor (Schmidt et al., 1995, supra; Uehara et al., 1995, supra). HGF-Met also plays a role in liver regeneration, angiogenesis, and wound healing (Bussolino et al., J Cell Biol (1992), 119, 629-641; Matsumoto and Nakamura, Exs (1993), 65, 225-249; Nusrat et al., J Clin Invest (1994) 93, 2056-2065). The precursor Met receptor undergoes proteolytic cleavage into an extracellular α subunit and membrane spanning β subunit linked by disulfide bonds (Tempest et al., Br J Cancer (1988), 58, 3-7). The β subunit contains the cytoplasmic kinase domain and harbors a multi-substrate docking site at the C-terminus where adapter proteins bind and initiate signaling (Bardelli et al., Oncogene (1997), 15, 3103-3111; Nguyen et al., J Biol Chem (1997), 272, 20811-20819; Pelicci et al., Oncogene (1995), 10, 1631-1638; Ponzetto et al., Cell (1994), 77, 261-271; Weidner et al., Nature (1996), 384, 173-176). Upon HGF binding, activation of Met leads to tyrosine phosphorylation and downstream signaling through Gab1 and Grb2/Sos mediated PI3-kinase and Ras/MAPK activation respectively, which drives cell motility and proliferation (Furge et al., Oncogene (2000), 19, 5582-5589; Hartmann et al., J Biol Chem (1994), 269, 21936-21939; Ponzetto et al., J Biol Chem (1996), 271, 14119-14123; Royal and Park, J Biol Chem (1995), 270, 27780-27787).

Met was shown to be transforming in a carcinogen-treated osteosarcoma cell line (Cooper et al., Nature (1984), 311, 29-33; Park et al., Cell (1986), 45, 895-904). Met overexpression or gene-amplification has been observed in a variety of human cancers. For example, Met protein is overexpressed at least 5-fold in colorectal cancers and reported to be gene-amplified in liver metastasis (Di Renzo et al., Clin Cancer Res (1995), 1, 147-154; Liu et al., Oncogene (1992), 7, 181-185). Met protein is also reported to be overexpressed in oral squamous cell carcinoma, hepatocellular carcinoma, renal cell carcinoma, breast carcinoma, and lung carcinoma (Jin et al., Cancer (1997), 79, 749-760; Morello et al., J Cell Physiol (2001), 189, 285-290; Natali et al., Int J Cancer (1996), 69, 212-217; Olivero et al., Br J Cancer (1996), 74, 1862-1868; Suzuki et al., Br J Cancer (1996), 74, 1862-1868). In addition, overexpression of mRNA has been observed in hepatocellular carcinoma, gastric carcinoma, and colorectal carcinoma (Boix et al., Hepatology (1994), 19, 88-91; Kuniyasu et al., Int J Cancer (1993), 55, 72-75; Liu et al., Oncogene (1992), 7, 181-185).

A number of mutations in the kinase domain of Met have been found in renal papillary carcinoma which leads to constitutive receptor activation (Olivero et al., Int J Cancer (1999), 82, 640-643; Schmidt et al., Nat Genet (1997), 16, 68-73; Schmidt et al., Oncogene (1999), 18, 2343-2350). These activating mutations confer constitutive Met tyrosine phosphorylation and result in MAPK activation, focus formation, and tumorigenesis (Jeffers et al., Proc Natl Acad Sci USA (1997), 94, 11445-11450). In addition, these mutations enhance cell motility and invasion (Giordano et al., Faseb J (2000), 14, 399-406; Lorenzato et al., Cancer Res (2002), 62, 7025-7030). HGF-dependent Met activation in transformed cells mediates increased motility, scattering, and migration which eventually leads to invasive tumor growth and metastasis (Jeffers et al., Mol Cell Biol (1996), 16, 1115-1125; Meiners et al., Oncogene (1998), 16, 9-20).

Met has been shown to interact with other proteins that drive receptor activation, transformation, and invasion. In neoplastic cells, Met is reported to interact with α6β4 integrin, a receptor for extracellular matrix (ECM) components such as laminins, to promote HGF-dependent invasive growth (Trusolino et al., Cell (2001), 107, 643-654). In addition, the extracellular domain of Met has been shown to interact with a member of the semaphorin family, plexin B1, and to enhance invasive growth (Giordano et al., Nat Cell Biol (2002), 4, 720-724). Furthermore, CD44v6, which has been implicated in tumorigenesis and metastasis, is also reported to form a complex with Met and HGF and result in Met receptor activation (Orian-Rousseau et al., Genes Dev (2002), 16, 3074-3086).

Met is a member of the subfamily of receptor tyrosine kinases (RTKs) which include Ron and Sea (Maulik et al., Cytokine Growth Factor Rev (2002), 13, 41-59). Prediction of the extracellular domain structure of Met suggests shared homology with the semaphorins and plexins. The N-terminus of Met contains a Sema domain of approximately 500 amino acids that is conserved in all semaphorins and plexins. The semaphorins and plexins belong to a large family of secreted and membrane-bound proteins first described for their role in neural development (Van Vactor and Lorenz, Curr Bio (1999), 19, R201-204). However, more recently semaphorin overexpression has been correlated with tumor invasion and metastasis. A cysteine-rich PSI domain (also referred to as a Met Related Sequence domain) found in plexins, semaphorins, and integrins lies adjacent to the Sema domain followed by four IPT repeats that are immunoglobulin-like regions found in plexins and transcription factors. A recent study suggests that the Met Sema domain is sufficient for HGF and heparin binding (Gherardi et al., Proc Natl Acad Sci USA (2003), 100(21):12039-44).

As noted above, the Met receptor tyrosine kinase is activated by its cognate ligand HGF and receptor phosphorylation activates downstream pathways of MAPK, PI-3 kinase and PLC-γ (L. Trusolino and P. M. Comoglio, Nat Rev Cancer 2, 289 (2002); C. Birchmeier et al., Nat Rev Mol Cell Biol 4, 915 (2003)). Phosphorylation of Y1234/Y1235 within the kinase domain is critical for Met kinase activation while Y1349 and Y1356 in the multisubstrate docking site are important for binding of src homology-2 (SH2), phosphotyrosine binding (PTB), and Met binding domain (MBD) proteins (C. Ponzetto et al., Cell 77, 261 (1994); K. M. Weidner et al., Nature 384, 173 (1996); G. Pelicci et al., Oncogene 10, 1631 (1995)) to mediate activation of downstream signaling pathways. An additional juxtamembrane phosphorylation site, Y1003, has been well characterized for its binding to the tyrosine kinase binding (TKB) domain of the Cb1 E3-ligase (P. Peschard et al., Mol Cell 8, 995 (2001); P. Peschard, N. Ishiyama, T. Lin, S. Lipkowitz, M. Park, J Biol Chem 279, 29565 (2004)). Cb1 binding is reported to drive endophilin-mediated receptor endocytosis, ubiquitination, and subsequent receptor degradation (A. Petrelli et al., Nature 416, 187 (2002)). This mechanism of receptor downregulation has been described previously in the EGFR family that also harbor a similar Cb1 binding site (K. Shtiegman, Y. Yarden, Semin Cancer Biol 13, 29 (2003); M. D. Marmor, Y. Yarden, Oncogene 23, 2057 (2004); P. Peschard, M. Park, Cancer Cell 3, 519 (2003)). Dysregulation of Met and HGF have been reported in a variety of tumors. Ligand-driven Met activation has been observed in several cancers. Elevated serum and intra-tumoral HGF is observed in lung, breast cancer, and multiple myeloma (J. M. Siegfried et al., Ann Thorac Surg 66, 1915 (1998); P. C. Ma et al., Anticancer Res 23, 49 (2003); B. E. Elliott et al. Can J Physiol Pharmacol 80, 91 (2002); C. Seidel, et al, Med Oncol 15, 145 (1998)). Overexpression of Met and/or HGF, Met amplification or mutation has been reported in various cancers such as colorectal, lung, gastric, and kidney cancer and is thought to drive ligand-independent receptor activation (C. Birchmeier et al, Nat Rev Mol Cell Biol 4, 915 (2003); G. Maulik et al., Cytokine Growth Factor Rev 13, 41 (2002)). Additionally, inducible overexpression of Met in a liver mouse model gives rise to hepatocellular carcinoma demonstrating that receptor overexpression drives ligand independent tumorigenesis (R. Wang, et al, J Cell Biol 153, 1023 (2001)). The most compelling evidence implicating Met in cancer is reported in familial and sporadic renal papillary carcinoma (RPC) patients. Mutations in the kinase domain of Met that lead to constitutive activation of the receptor were identified as germline and somatic mutations in RPC (L. Schmidt et al., Nat Genet 16, 68 (1997)). Introduction of these mutations in transgenic mouse models leads to tumorigenesis and metastasis. (M. Jeffers et al., Proc Natl Acad Sci USA 94, 11445 (1997)).

The epidermal growth factor receptor (EGFR) family comprises four closely related receptors (HER1/EGFR, HER2, HER3 and HER4) involved in cellular responses such as differentiation and proliferation. Over-expression of the EGFR kinase, or its ligand TGF-alpha, is frequently associated with many cancers, including breast, lung, colorectal, ovarian, renal cell, bladder, head and neck cancers, glioblastomas, and astrocytomas, and is believed to contribute to the malignant growth of these tumors. A specific deletion-mutation in the EGFR gene (EGFRvIII) has also been found to increase cellular tumorigenicity. Activation of EGFR stimulated signaling pathways promote multiple processes that are potentially cancer-promoting, e.g. proliferation, angiogenesis, cell motility and invasion, decreased apoptosis and induction of drug resistance. Increased HER1/EGFR expression is frequently linked to advanced disease, metastases and poor prognosis. For example, in NSCLC and gastric cancer, increased HER1/EGFR expression has been shown to correlate with a high metastatic rate, poor tumor differentiation and increased tumor proliferation.

Mutations which activate the receptor's intrinsic protein tyrosine kinase activity and/or increase downstream signaling have been observed in NSCLC and glioblastoma. However the role of mutations as a principle mechanism in conferring sensitivity to EGF receptor inhibitors, for example erlotinib (TARCEVA®) or gefitinib, has been controversial. Mutant forms of the full length EGF receptor has been reported to predict responsiveness to the EGF receptor tyrosine kinase inhibitor gefitinib (Paez, J. G. et al. (2004) Science 304:1497-1500; Lynch, T. J. et al. (2004) N. Engl. J. Med. 350:2129-2139). Cell culture studies have shown that cell lines which express such mutant forms of the EGF receptor (i.e. H3255) were more sensitive to growth inhibition by the EGF receptor tyrosine kinase inhibitor gefitinib, and that much higher concentrations of gefitinib was required to inhibit the tumor cell lines expressing wild type EGF receptor. These observations suggests that specific mutant forms of the EGF receptor may reflect a greater sensitivity to EGF receptor inhibitors, but do not identify a completely non-responsive phenotype.

The development for use as anti-tumor agents of compounds that directly inhibit the kinase activity of the EGFR, as well as antibodies that reduce EGFR kinase activity by blocking EGFR activation, are areas of intense research effort (de Bono J. S. and Rowinsky, E. K. (2002) Trends in Mol. Medicine. 8:S19-S26; Dancey, J. and Sausville, E. A. (2003) Nature Rev. Drug Discovery 2:92-313). Several studies have demonstrated, disclosed, or suggested that some EGFR kinase inhibitors might improve tumor cell or neoplasia killing when used in combination with certain other anti-cancer or chemotherapeutic agents or treatments (e.g. Herbst, R. S. et al. (2001) Expert Opin. Biol. Ther. 1:719-732; Solomon, B. et al (2003) Int. J. Radiat. Oncol. Biol. Phys. 55:713-723; Krishnan, S. et al. (2003) Frontiers in Bioscience 8, e1-13; Grunwald, V. and Hidalgo, M. (2003) J. Nat. Cancer Inst. 95:851-867; Seymour L. (2003) Current Opin. Investig. Drugs 4(6):658-666; Khalil, M. Y. et al. (2003) Expert Rev. Anticancer Ther. 3:367-380; Bulgaru, A. M. et al. (2003) Expert Rev. Anticancer Ther. 3:269-279; Dancey, J. and Sausville, E. A. (2003) Nature Rev. Drug Discovery 2:92-313; Ciardiello, F. et al. (2000) Clin. Cancer Res. 6:2053-2063; and Patent Publication No: US 2003/0157104).

Erlotinib (e.g. erlotinib HCl, also known as TARCEVA® or OSI-774) is an orally available inhibitor of EGFR kinase. In vitro, erlotinib has demonstrated substantial inhibitory activity against EGFR kinase in a number of human tumor cell lines, including colorectal and breast cancer (Moyer J. D. et al. (1997) Cancer Res. 57:4838), and preclinical evaluation has demonstrated activity against a number of EGFR-expressing human tumor xenografts (Pollack, V. A. et al (1999) J. Pharmacol. Exp. Ther. 291:739). Erlotinib has demonstrated activity in clinical trials in a number of indications, including head and neck cancer (Soulieres, D., et al. (2004) J. Clin. Oncol. 22:77), NSCLC (Perez-Soler R, et al. (2001) Proc. Am. Soc. Clin. Oncol. 20:310a, abstract 1235), CRC (Oza, M., et al. (2003) Proc. Am. Soc. Clin. Oncol. 22:196a, abstract 785) and MBC (Winer, E., et al. (2002) Breast Cancer Res. Treat. 76:5115a, abstract 445; Jones, R. J., et al. (2003) Proc. Am. Soc. Clin. Oncol. 22:45a, abstract 180). In a phase III trial, erlotinib monotherapy significantly prolonged survival, delayed disease progression and delayed worsening of lung cancer-related symptoms in patients with advanced, treatment-refractory NSCLC (Shepherd, F. et al. (2004) J. Clin. Oncology, 22:14 S (July 15 Supplement), Abstract 7022). In November 2004 the U.S. Food and Drug Administration (FDA) approved TARCEVA® for the treatment of patients with locally advanced or metastatic non-small cell lung cancer (NSCLC) after failure of at least one prior chemotherapy regimen.

Despite the significant advancement in the treatment of cancer, improved therapies are still being sought.

All references cited herein, including patent applications and publications, are incorporated by reference in their entirety.

### SUMMARY OF THE INVENTION

The present invention provides therapies for treating a pathological condition, such as cancer, wherein an anti-c-met antibody provides significant anti-tumor activity. The present invention also provides combination therapies for treating a pathological condition, such as cancer, wherein an anti-c-met antibody is combined with an EGFR antagonist, thereby providing significant anti-tumor activity.

In one aspect, the invention provides methods of treating cancer in a subject, comprising administering to the subject an anti-c-met antibody at a dose of about 15 mg/kg every three weeks.

In another aspect, the invention provides methods of treating cancer in a subject, comprising administering to the subject (a) an anti-c-met antibody at a dose of about 15 mg/kg every three weeks; and (b) an EGFR antagonist.

In one aspect, the invention provides methods for extending time to disease progression (TTP) or survival in a subject with non-small cell lung cancer, the method comprising administering to the subject (a) an anti-c-met antibody at a dose of about 15 mg/kg every three weeks; and (b) an EGFR antagonist.

In some embodiments, the anti-c-met antibody is administered in an amount sufficient to achieve a serum trough concentration at or above 15 micrograms/ml. In some embodiments, the anti-c-met antibody is administered at a total dose of about 15 mg/kg over a three week period.

In one embodiment, the EGFR antagonist is erlotinib. In certain embodiments, erlotinib is administered at a dose of 150 mg, each day of a three week cycle. In certain embodiments, erlotinib is administered at a dose of 100 mg, each day of a three week cycle. In certain embodiments, erlotinib is administered at a dose of 50 mg, each day of a three week cycle.

In one embodiment, the invention provides methods for extending time to disease progression (TTP), progression-free survival, or survival in a subject with non-small cell lung cancer, the method comprising administering to the subject (a) an anti-c-met antibody (such as MetMAb) at a dose of about 15 mg/kg every three weeks; and (b) erlotinib (N-(3-ethynylphenyl)-6,7-bis(2-methoxyethoxy)-4-quinazolinamine) at a dose of 150 mg, each day of a three week cycle.

The present application discloses administration in humans for the first time of a monovalent one-armed antibody comprising a Fc region that increases stability of said antibody fragment compared to a Fab molecule comprising said antigen binding arm. See, e.g., WO2005/063816. A full length antibody may in some cases exhibit agonistic effects (which may be undesirable) upon binding to a target antigen even though it is an antagonistic antibody as a Fab fragment. See, e.g., U.S. Pat. No. 6,468,529. This phenomenon is unfortunate where the antagonistic effect is the desired therapeutic function. The monovalent trait of a one-armed antibody (i.e., an antibody comprising a single antigen binding arm) results in and/or ensures an antagonistic function upon binding of the antibody to a target molecule, suitable for treatment of pathological conditions requiring an antagonistic function and where bivalency of an antibody results in an undesirable agonistic effect. Furthermore, the one-armed antibody comprising the Fc region as described herein is characterized by superior pharmacokinetic attributes (such as an enhanced half life and/or reduced clearance rate in vivo) compared to Fab forms having similar/substantially identical antigen binding characteristics, thus overcoming a major drawback in the use of conventional monovalent Fab antibodies.

Accordingly, in some embodiment, the anti-c-met antibody is a one-armed antibody (i.e., the heavy chain variable domain and the light chain variable domain form a single antigen binding arm) comprising an Fc region, wherein the Fc region comprises a first and a second Fc polypeptide, wherein the first and second Fc polypeptides are present in a complex and form a Fc region that increases stability of said antibody fragment compared to a Fab molecule comprising said antigen binding arm.

In some embodiments, the anti-c-met antibody comprises (a) a first polypeptide comprising a heavy chain variable domain having the sequence: EVQLVESGGGLVQPGGSLRLSCAASGYTFTSYWLHWVRQAPGKGLEWVGMIDPSNSDTRFN PNFKDRFTISADTSKNTAYLQMNSLRAEDTAVYYCATYRSYVTPLDYWGQGTLVTVSS (SEQ ID NO:10), CH1 sequence, and a first Fc polypeptide; (b) a second polypeptide comprising a light chain variable domain having the sequence: DIQMTQSPSSLSASVGDRVTITCKSSQSLLYTSSQKNYLAWYQQKPGKAPKLLIYWASTR ESGVPSRFSGSGSGTDFTLTISSLQPEDFATYYCQQYYAYPWTFGQGTKVEIKR (SEQ ID NO:11), and CL1 sequence; and (c) a third polypeptide comprising a second Fc polypeptide, wherein the heavy chain variable domain and the light chain variable domain are present as a complex and form a single antigen binding arm, wherein the first and second Fc polypeptides are present in a complex and form a Fc region that increases stability of said antibody fragment compared to a Fab molecule comprising said antigen binding arm. In some embodiments, the first polypeptide comprises the Fc sequence depicted in FIG. 1 (SEQ ID NO: 12) and the second polypeptide comprises the Fc sequence depicted in FIG. 2 (SEQ ID NO: 13). In some embodiments, the first polypeptide comprises the Fc sequence depicted in FIG. 2 (SEQ ID NO: 13) and the second polypeptide comprises the Fc sequence depicted in FIG. 1 (SEQ ID NO: 12).

In some embodiments, the anti-c-met antibody comprises (a) a first polypeptide comprising a heavy chain variable domain, said polypeptide comprising the sequence: EVQLVESGGGLVQPGGSLRLSCAASGYTFTSYWLHWVRQAPGKGLEWVGMIDPSNSDTRFN PNFKDRFTISADTSKNTAYLQMNSLRAEDTAVYYCATYRSYVTPLDYWGQGTLVTVS SAST KGPSVFPLAPSSKSTSGGTAALGCLVKDYFPEPVTVSWNSGALTSGVHTFPAVLQSSGLYSLS SVVTVPSSSLGTQTYICNVNHKPSNTKVDKKVEPKSCDKTHTCPPCPAPELLGGPSVFLFPPKP KDTLMISRTPEVTCVVVDVSHEDPEVKFNWYVDGVEVHNAKTKPREEQYNSTYRVVSVLTV LHQDWLNGKEYKCKVSNKALPAPIEKTISKAKGQPREPQVYTLPPSREEMTKNQVSLSCAVK GFYPSDIAVEWESNGQPENNYKTTPPVLDSDGSFFLVSKLTVDKSRWQQGNVFSCSVMHEAL HNHYTQKSLSLSPGK (SEQ ID NO: 14); (b) a second polypeptide comprising a light chain variable domain, the polypeptide comprising the sequence DIQMTQSPSSLSASVGDRVTITCKSSQSLLYTSSQKNYLAWYQQKPGKAPKLLIYWASTRESG VPSRFSGSGSGTDFTLTISSLQPEDFATYYCQQYYAYPWTFGQGTKVEIKRTVAAPSVFIFPPS DEQLKSGTASVVCLLNNFYPREAKVQWKVDNALQSGNSQESVTEQDSKDSTYSLSSTLTLSK ADYEKHKVYACEVTHQGLSSPVTKSFNRGEC (SEQ ID NO:15); and a third polypeptide comprising a FC sequence, the polypeptide comprising the sequence CPPCPAPELLGGPSVFLFPPKPKDTLMISRTPEVTCVVVDVSHEDPEVKFNWYVDGVEVHNA KTKPREEQYNSTYRVVSVLTVLHQDWLNGKEYKCKVSNKALPAPIEKTISKAKGQPREPQV YTLPPSREEMTKNQVSLWCLVKGFYPSDIAVEWESNGQPENNYKTTPPVLDSDGSFFLYSKL TVDKSRWQQGNVFSCSVMHEALHNHYTQKSLSLSPGK (SEQ ID NO: 13), wherein the heavy chain variable domain and the light chain variable domain are present as a complex and form a single antigen binding arm, wherein the first and second Fc polypeptides are present in a complex and form a Fc region that increases stability of said antibody fragment compared to a Fab molecule comprising said antigen binding arm.

In one embodiment, the anti-c-met antibody comprises a heavy chain variable domain comprising one or more of CDR1-HC, CDR2-HC and CDR3-HC sequence depicted in FIG. 1 (SEQ ID NO: 4, 5, and/or 9). In some embodiments, the antibody comprises a light chain variable domain comprising one or more of CDR1-LC, CDR2-LC and CDR3-LC sequence depicted in FIG. 1 (SEQ ID NO: 1, 2, and/or 3). In some embodiments, the heavy chain variable domain comprises FR1-HC, FR2-HC, FR3-HC and FR4-HC sequence depicted in FIG. 1 (SEQ ID NO: 21-24). In some embodiments, the light chain variable domain comprises FR1-LC, FR2-LC, FR3-LC and FR4-LC sequence depicted in FIG. 1 (SEQ ID NO: 16-19).

Other anti-c-met antibodies suitable for use in the methods of the invention are described herein and known in the art.

In one aspect, the anti-c-met antibody comprises at least one characteristic that promotes heterodimerization, while minimizing homodimerization, of the Fc sequences within the antibody fragment. Such characteristic(s) improves yield and/or purity and/or homogeneity of the immunoglobulin populations. In one embodiment, the antibody comprises Fc mutations constituting “knobs” and “holes” as described in WO2005/063816. For example, a hole mutation can be one or more of T366A, L368A and/or Y407V in an Fc polypeptide, and a cavity mutation can be T366W.

Methods of the invention can be used to affect any suitable pathological state. For example, methods of the invention can be used for treating different cancers, both solid tumors and soft-tissue tumors alike. Non-limiting examples of cancers amendable to the treatment of the invention include breast cancer, colorectal cancer, rectal cancer, non-small cell lung cancer, non-Hodgkins lymphoma (NHL), renal cell cancer, prostate cancer, liver cancer, pancreatic cancer, soft-tissue sarcoma, kaposi's sarcoma, carcinoid carcinoma, head and neck cancer, melanoma, ovarian cancer, gastric cancer, mesothelioma, and multiple myeloma. In certain aspects, the cancers are metastatic. In other aspects, the cancers are non-metastatic.

In some embodiments, the cancer is non-small cell lung cancer, renal cell cancer, pancreatic cancer, gastric carcinoma, bladder cancer, esophageal cancer, mesothelioma, melanoma, breast cancer, thyroid cancer, colorectal cancer, head and neck cancer, osteosarcoma, prostate cancer, or glioblastoma.

In some embodiments, the subject's cancer expresses c-met. In some embodiments, the subject's cancer expresses EGFR. In some embodiments, the subject's cancer displays c-met and/or EGFR expression, amplification, or activation.

In some embodiments, serum from a subject expresses high levels of IL8 (displays high levels of IL8 expression, such as IL8 protein expression). In some embodiments, serum from a subject expresses greater than about 150 pg/ml of IL8, or in some embodiments, greater than about 50 pg/ml IL8. In some embodiments, serum from a subject expresses greater than about 10 pg/ml, 20 pg/ml, 30 pg/ml or more of IL8. Methods for determining IL8 serum concentration are known in the art and one method is described in the present Examples.

In some embodiments, serum from a subject expresses high levels of HGF (displays high level of HGF expression, such as HGF protein expression). In some embodiments, serum from a subject expresses greater than about 5,000, 10,000, or 50,000 pg/ml of HGF.

The anti-c-met antibody can be administered serially or in combination with the EGFR antagonist, either in the same composition or as separate compositions. The administration of the anti-c-met antibody and the EGFR antagonist can be done simultaneously, e.g., as a single composition or as two or more distinct compositions, using the same or different administration routes. Alternatively, or additionally, the administration can be done sequentially, in any order. Alternatively, or additionally, the steps can be performed as a combination of both sequentially and simultaneously, in any order. In certain embodiments, intervals ranging from minutes to days, to weeks to months, can be present between the administrations of the two or more compositions. For example, the EGFR antagonist may be administered first, followed by the anti-c-met antibody. However, simultaneous administration or administration of the anti-c-met antibody first is also contemplated.

Depending on the specific cancer indication to be treated, the combination therapy of the invention can be combined with additional therapeutic agents, such as chemotherapeutic agents, VEGF antagonists, or additional therapies such as radiotherapy or surgery. Many known chemotherapeutic agents can be used in the combination therapy of the invention. Preferably those chemotherapeutic agents that are standard for the treatment of the specific indications will be used. Dosage or frequency of each therapeutic agent to be used in the combination is preferably the same as, or less than, the dosage or frequency of the corresponding agent when used without the other agent(s).

The invention also provides prognostic methods. Therefore, the disclosed methods can provide for convenient, efficient, and potentially cost-effective means to obtain data and information useful in assessing future course of the disorder, including selection of appropriate therapies for treating patients.

In another aspect, the invention provides methods for evaluation of a patient having or suspected of having cancer, the method comprising: predicting cancer prognosis of the patient based on a comparison of expression of IL8 in a biological sample from the patient with expression of IL8 in a control sample; wherein IL8 expression in the patient biological sample relative to a control sample is prognostic for cancer in the patient. In some embodiments, the method further comprises (a) obtaining biological sample from the patient (e.g., prior to and/or during treatment); and (b) detecting IL8 expression in the biological sample(s). In some embodiments, increased IL8 expression in the patient biological sample relative to the control sample is prognostic for cancer in the patient. In some embodiments, decreased IL8 expression in the patient biological sample relative to the control sample is prognostic for cancer in the patient.

In another aspect, the invention provides methods for evaluation of a patient undergoing treatment for cancer, the method comprising: predicting cancer prognosis of the patient based on a comparison of expression of IL8 in a biological sample (e.g., serum) from the patient with expression of IL8 in the patient biological sample taken prior to treatment, wherein decreased IL8 expression in the serum of the patient undergoing treatment relative to expression in the pre-treatment sample is prognostic for cancer in the patient.

In some embodiments, prognostic for cancer comprises providing the forecast or prediction of (prognostic for) any one or more of the following: response to treatment (e.g., with c-met antagonist (such as an anti-c-met antibody) or with c-met antagonist and EGFR antagonist), activity of c-met antagonist (such as an anti-c-met antibody) or c-met antagonist and EGFR antagonist, response to treatment (e.g., with a c-met antagonist or with a c-met antagonist and an EGFR antagonist), activity of treatment (e.g., with a c-met antagonist or with a c-met antagonist and an EGFR antagonist), duration of survival of a patient susceptible to or diagnosed with a cancer, duration of recurrence-free survival, duration of progression free survival of a patient susceptible to or diagnosed with a cancer, response rate in a group of patients susceptible to or diagnosed with a cancer, duration of response in a patient or a group of patients susceptible to or diagnosed with a cancer, and/or likelihood of metastasis in a patient susceptible to or diagnosed with a cancer. In some embodiments, duration of survival is forecast or predicted to be increased. In some embodiment, duration of survival is forecast or predicted to be decreased. In some embodiments, duration of recurrence-free survival is forecast or predicted to be increased. In some embodiment, duration of recurrence-free survival is forecast or predicted to be decreased. In some embodiments, response rate is forecast or predicted to be increased. In some embodiments, response rate is forecast or predicted to be decreased. In some embodiments, duration of response is predicted or forecast to be increased. In some embodiments, duration of response is predicted or forecast to be decreased. In some embodiments, likelihood of metastasis is predicted or forecast to be increased. In some embodiments, likelihood of metastasis is predicted or forecast to be decreased.

In another aspect, the invention provides methods for selection of treatment for a patient having or suspected of having cancer, the methods comprising: (a) predicting cancer prognosis of the patient based on a comparison of expression of IL8 in a biological sample from the patient with expression of IL8 in a control sample, wherein IL8 expression in the patient biological sample relative to the control sample is prognostic for cancer in the patient, and (b) subsequent to step (a), selecting cancer treatment for the patient, wherein the selection of treatment is based on the patient prognosis determined in step (a). In some embodiments, the methods further comprise (c) obtaining a patient biological sample; (d) detecting IL8 expression in the biological sample, wherein IL8 expression in the patient biological sample is prognostic of cancer. In some embodiments, increased IL8 expression in the patient biological sample relative to the control sample is prognostic for cancer in the patient. In some embodiments, decreased IL8 expression in the patient biological sample relative to the control sample is prognostic for cancer in the patient.

In another aspect, the invention provides methods for selection of treatment for a patient undergoing treatment for cancer, the methods comprising: (a) predicting cancer prognosis of the patient based on a comparison of expression of IL8 in a biological sample (e.g., serum) from the patient with expression of IL8 in the patient biological sample taken prior to treatment, wherein IL8 expression in the serum of a patient undergoing treatment relative to expression in the pre-treatment sample is prognostic for cancer in the patient is prognostic for cancer in the patient, and (b) subsequent to step (a), selecting cancer treatment for the patient, wherein the selection of treatment is based on the patient prognosis determined in step (a). In some embodiments, the methods further comprise (c) obtaining a patient biological sample; (d) detecting IL8 expression in the biological sample, wherein IL8 expression in the patient biological sample is prognostic of cancer. In some embodiments, increased IL8 expression in the patient biological sample relative to the control sample is prognostic for cancer in the patient. In some embodiments, decreased IL8 expression in the patient biological sample relative to the control sample is prognostic for cancer in the patient.

### BRIEF DESCRIPTION OF THE DRAWINGS

FIG. 1: depicts amino acid sequences of the framework (FR), CDR, first constant domain (CL or CH1) and Fc region (Fc) of MetMAb (OA5D5v2). The Fc sequence depicted comprises “hole” (cavity) mutations T366S, L368A and Y407V, as described in WO 2005/063816.

FIG. 2: depicts sequence of an Fc polypeptide comprising “knob” (protuberance) mutation T366W, as described in WO 2005/063816. In one embodiment, an Fc polypeptide comprising this sequence forms a complex with an Fc polypeptide comprising the Fc sequence of FIG. 1 to generate an Fc region.

FIG. 3: Mean serum MetMAb concentration-time profiles following a single IV or IP bolus dose of MetMAb in mice, rats, and cynomolgus monkeys. MetMAb was dosed on day 0 as indicated.

FIG. 4: Mean tumor volume-time profiles following a single IV bolus dose of MetMAb at multiple dose levels in KP4 pancreatic cancer xenograft model. MetMAb was dosed on day 0 as indicated.

FIG. 5: Mean tumor volume-dose profile. Group mean tumor volume on Day 21=Σ(tumor volume Day 21−the same animal's tumor volume Day 0)/n.

FIG. 6: Mean tumor volume-time profiles following an IV bolus dose of MetMAb with different dosing regimens in KP4 xenograft model. Arrows indicate the time of dosing for dose groups as follows (from top row to bottom row): top arrow: MetMAb 0.825 mg/kg once per week (Q1W); middle arrow: MetMAb 1.25 mg/kg once every two weeks (Q2W); bottom arrow: MetMAb 2.5 mg/kg once every three weeks (Q3W). “OA5D5” refers to MetMAb in this figure. Diamond shape indicates PBS control.

FIG. 7: Mean tumor volume-time profiles following a single IV bolus dose or IV infusion of MetMAb in KP4 xenograft model. MetMAb was dosed as indicated.

FIG. 8: Mean tumor volume-time profiles following an IV bolus dose in H596 non-small cell lung cancer tumor-bearing huHGF-SCID transgenic mice. MetMAb was dosed as indicated.

FIG. 9: Illustration of a theoretical human population PK/PD model of tumor progression for MetMAb comprised of a two-compartment nonlinear PK model with direct KP4 tumor growth inhibition. CL=non-saturable clearance component of total clearance; CLd=inter-compartmental clearance; Kim₁₀=MetMAb serum concentration at 50% Vₘₐₓ₁₀; Vₘₐₓ₁₀=maximum drug removal for the saturable clearance component of total clearance; V1=Apparent central volume of distribution; V2=Apparent peripheral volume of distribution; IC₅₀=Michaelis-Menten constant representing the MetMAb serum concentration leading to 50% cell growth inhibition; IMax=maximal MetMAb tumor growth inhibition effect constant; KGN=in vivo net growth rate of the KP4 tumor cell line; C=MetMAb serum concentration.

FIG. 10: Representative PK profiles and MTC values from 15 mg/kg Q3W MetMAb simulations. PK=pharmacokinetic profile; MTC=minimum tumorostatic MetMAb concentration.

FIG. 11: Tumor mass simulations corresponding to the PK profiles and MTC values shown in FIG. 10. AUC=MetMAb serum area under the curve; MTC=minimum tumorostatic MetMAb concentration.

FIG. 12: Phase I dose escalation study design.

FIG. 13: Patient diagnosis, treatment cohort and administered cycles in the Phase I dose escalation study. Cycles of MetMAb exposure for each patient in the dose-escalation stage. Unless otherwise noted, all patients came off study for progressive disease.

FIG. 14: MetMAb serum concentrations at each pharmacokinetic timepoint were averaged across all patients in each dose group. The mean (±SD) MetMAb concentrations are plotted versus time for each cohort.

FIG. 15: PK/PD modeling was used to determine the median MTC in humans, based on preclinical tumor xenograft studies and interspecies scaling. The MTC of MetMAb in serum in humans was determined to be 15 ug/mL. Simulations based on observed PK data from this Phase 1 study were used to identify the 15 mg/kg Q3W dose (arrow) that achieves steady-state trough concentrations greater than or equal to MTC in 90% of patients. MTC=minimum tumorostatic concentration, PK=pharmacokinetics, PD=pharmacodynamic; SS=steady state, Q3W=once every 3 weeks.

FIG. 16: Inhibition of Met may affect circulating ligand HGF levels. Serum HGF levels were determined by an ELISA based method. Data are presented in descending order of baseline HGF expression. In general, there appears to be little or no increase in HGF expression with MetMAb treatment. However, two patients who exhibited the highest levels of baseline HGF expression showed significantly decreased HGF expression. For patient 11009, HGF levels decreased by 70% post-drug treatment and remained low. C=cycle, D=day, HGF=hepatocyte growth factor, M=male, F=female, C1D1, C2D1, C3D1: pre-dose; C1D2: 24 h post-dose.

FIG. 17: Evaluation of serum IL8 levels. Data are presented in descending order of baseline IL8 expression. In general, most patients with baseline serum IL8 levels above normal controls had a decrease in serum IL8 following MetMAb infusions. Intrasubject variability in IL8 in healthy volunteers over a period of 4 weeks was ˜3-10 pg/ml. IL8=Interleukin 8, MSD=meso scale discovery, C=cycle, D=day, M=male, F=female, C1D1, C2D1, C3D1: pre-dose; C1D2: 24 h post-dose.

FIG. 18: Best tumor response of all the patients who participated in the dose escalation stage. One patient was not assessed as the patient progressed before the first evaluation timepoint; another patient's CT evaluation was not available at the time these data were collected. Patient number and type of tumor are indicated. SLD=sum of the longest diameter.

FIG. 19: CT and MRI scans of patient 11009. Upper left panel: CT scan of pt11009 in August 2007. Upper right panel: CT scan of pt11009, which qualified her for enrollment into the MetMAb Phase I trial. Lower left panel: CT scan showing complete response. Lower right panel: MRI scan confirming complete response. The circle indicates the site of tumor.

FIG. 20: Immunohistochemical staining of archival tissue from patient 11009. Immunohistochemical staining of an archival gastric adenocarcinoma specimen from pt 11009 revealed moderate membraneous and cytoplasmic c-met expression and cytoplasmic and peri-membraneous HGF expression in tumor cells

### DETAILED DESCRIPTION

### I. Definitions

The term “hepatocyte growth factor” or “HGF”, as used herein, refers, unless indicated otherwise, to any native or variant (whether native or synthetic) HGF polypeptide that is capable of activating the HGF/c-met signaling pathway under conditions that permit such process to occur. The term “wild type HGF” generally refers to a polypeptide comprising the amino acid sequence of a naturally occurring HGF protein. The term “wild type HGF sequence” generally refers to an amino acid sequence found in a naturally occurring HGF. C-met is a known receptor for HGF through which HGF intracellular signaling is biologically effectuated.

The term “HGF variant” as used herein refers to a HGF polypeptide which includes one or more amino acid mutations in the native HGF sequence. Optionally, the one or more amino acid mutations include amino acid substitution(s).

A “native sequence” polypeptide comprises a polypeptide having the same amino acid sequence as a polypeptide derived from nature. Thus, a native sequence polypeptide can have the amino acid sequence of naturally-occurring polypeptide from any mammal. Such native sequence polypeptide can be isolated from nature or can be produced by recombinant or synthetic means. The term “native sequence” polypeptide specifically encompasses naturally-occurring truncated or secreted forms of the polypeptide (e.g., an extracellular domain sequence), naturally-occurring variant forms (e.g., alternatively spliced forms) and naturally-occurring allelic variants of the polypeptide.

A polypeptide “variant” means a biologically active polypeptide having at least about 80% amino acid sequence identity with the native sequence polypeptide. Such variants include, for instance, polypeptides wherein one or more amino acid residues are added, or deleted, at the N- or C-terminus of the polypeptide. Ordinarily, a variant will have at least about 80% amino acid sequence identity, more preferably at least about 90% amino acid sequence identity, and even more preferably at least about 95% amino acid sequence identity with the native sequence polypeptide.

By “EGFR” is meant the receptor tyrosine kinase polypeptide Epidermal Growth Factor Receptor which is described in Ullrich et al, Nature (1984) 309:418425, alternatively referred to as Her-1 and the c-erbB gene product, as well as variants thereof such as EGFRvIII. Variants of EGFR also include deletional, substitutional and insertional variants, for example those described in Lynch et al (New England Journal of Medicine 2004, 350:2129), Paez et al (Science 2004, 304:1497), Pao et al (PNAS 2004, 101:13306).

A “biological sample” (interchangeably termed “sample” or “tissue or cell sample”) encompasses a variety of sample types obtained from an individual and can be used in a diagnostic or monitoring assay. The definition encompasses blood and other liquid samples of biological origin, solid tissue samples such as a biopsy specimen or tissue cultures or cells derived therefrom, and the progeny thereof. The definition also includes samples that have been manipulated in any way after their procurement, such as by treatment with reagents, solubilization, or enrichment for certain components, such as proteins or polynucleotides, or embedding in a semi-solid or solid matrix for sectioning purposes. The term “biological sample” encompasses a clinical sample, and also includes cells in culture, cell supernatants, cell lysates, serum, plasma, biological fluid, and tissue samples. The source of the biological sample may be solid tissue as from a fresh, frozen and/or preserved organ or tissue sample or biopsy or aspirate; blood or any blood constituents; bodily fluids such as cerebral spinal fluid, amniotic fluid, peritoneal fluid, or interstitial fluid; cells from any time in gestation or development of the subject. In some embodiments, the biological sample is obtained from a primary or metastatic tumor. The biological sample may contain compounds which are not naturally intermixed with the tissue in nature such as preservatives, anticoagulants, buffers, fixatives, nutrients, antibiotics, or the like.

An “anti-c-met antibody” is an antibody that binds to c-met with sufficient affinity and specificity. The antibody selected will normally have a sufficiently strong binding affinity for c-met, for example, the antibody may bind human c-met with a K_{d} value of between 100 nM-1 pM. Antibody affinities may be determined by a surface plasmon resonance based assay (such as the BIAcore assay as described in PCT Application Publication No. WO2005/012359); enzyme-linked immunoabsorbent assay (ELISA); and competition assays (e.g. RIA's), for example. In certain embodiments, the anti-c-met antibody can be used as a therapeutic agent in targeting and interfering with diseases or conditions wherein c-met activity is involved. Also, the antibody may be subjected to other biological activity assays, e.g., in order to evaluate its effectiveness as a therapeutic. Such assays are known in the art and depend on the target antigen and intended use for the antibody.

“C-met activation” refers to activation, or phosphorylation, of the c-met receptor. Generally, c-met activation results in signal transduction (e.g. that caused by an intracellular kinase domain of a c-met receptor phosphorylating tyrosine residues in c-met or a substrate polypeptide). C-met activation may be mediated by c-met ligand (HGF) binding to a c-met receptor of interest. HGF binding to c-met may activate a kinase domain of c-met and thereby result in phosphorylation of tyrosine residues in the c-met and/or phosphorylation of tyrosine residues in additional substrate polypeptides(s).

An “EGFR antagonist” (interchangeably termed “EGFR inhibitor”) is an agent that interferes with EGFR activation or function. Examples of EGFR inhibitors include EGFR antibodies; EGFR ligand antibodies; small molecule EGFR antagonists; EGFR tyrosine kinase inhibitors; antisense and inhibitory RNA (e.g., shRNA) molecules (see, for example, WO2004/87207). Preferably, the EGFR inhibitor is an antibody or small molecule which binds to EGFR. In some embodiments, the EGFR inhibitor is an EGFR-targeted drug. In a particular embodiment, an EGFR inhibitor has a binding affinity (dissociation constant) to EGFR of about 1,000 nM or less. In another embodiment, an EGFR inhibitor has a binding affinity to EGFR of about 100 nM or less. In another embodiment, an EGFR inhibitor has a binding affinity to EGFR of about 50 nM or less. In a particular embodiment, an EGFR inhibitor is covalently bound to EGFR. In a particular embodiment, an EGFR inhibitor inhibits EGFR signaling with an IC50 of 1,000 nM or less. In another embodiment, an EGFR inhibitor inhibits EGFR signaling with an IC50 of 500 nM or less. In another embodiment, an EGFR inhibitor inhibits EGFR signaling with an IC50 of 50 nM or less. In certain embodiments, the EGFR antagonist reduces or inhibits, by at least 10%, 20%, 30%, 40%, 50%, 60%, 70%, 80%, 90% or more, the expression level or biological activity of EGFR.

“EGFR activation” refers to activation, or phosphorylation, of EGFR. Generally, EGFR activation results in signal transduction (e.g. that caused by an intracellular kinase domain of EGFR receptor phosphorylating tyrosine residues in EGFR or a substrate polypeptide). EGFR activation may be mediated by EGFR ligand binding to a EGFR dimer comprising EGFR. EGFR ligand binding to a EGFR dimer may activate a kinase domain of one or more of the EGFR in the dimer and thereby results in phosphorylation of tyrosine residues in one or more of the EGFR and/or phosphorylation of tyrosine residues in additional substrate polypeptides(s).

As used herein, the term “EGFR-targeted drug” refers to a therapeutic agent that binds to EGFR and inhibits EGFR activation. Examples of such agents include antibodies and small molecules that bind to EGFR. Examples of antibodies which bind to EGFR include MAb 579 (ATCC CRL HB 8506), MAb 455 (ATCC CRL HB8507), MAb 225 (ATCC CRL 8508), MAb 528 (ATCC CRL 8509) (see, U.S. Pat. No. 4,943,533, Mendelsohn et al.) and variants thereof, such as chimerized 225 (C225 or Cetuximab; ERBUTIX®) and reshaped human 225 (H225) (see, WO 96/40210, Imclone Systems Inc.); IMC-11F8, a fully human, EGFR-targeted antibody (Imclone); antibodies that bind type II mutant EGFR (U.S. Pat. No. 5,212,290); humanized and chimeric antibodies that bind EGFR as described in U.S. Pat. No. 5,891,996; and human antibodies that bind EGFR, such as ABX-EGF (see WO98/50433, Abgenix); EMD 55900 (Stragliotto et al. Eur. J. Cancer 32A:636-640 (1996)); EMD7200 (matuzumab) a humanized EGFR antibody directed against EGFR that competes with both EGF and TGF-alpha for EGFR binding; and mAb 806 or humanized mAb 806 (Johns et al., J. Biol. Chem. 279(29):30375-30384 (2004)). The anti-EGFR antibody may be conjugated with a cytotoxic agent, thus generating an immunoconjugate (see, e.g., EP659,439A2, Merck patent GmbH). Examples of small molecules that bind to EGFR include ZD1839 or Gefitinib (IRESSA; Astra Zeneca); CP-358774 or Erlotinib (TARCEVA™; Genentech/OSI); and AG1478, AG1571 (SU 5271; Sugen); EMD-7200.

The phrase “gene amplification” refers to a process by which multiple copies of a gene or gene fragment are formed in a particular cell or cell line. The duplicated region (a stretch of amplified DNA) is often referred to as “amplicon.” Usually, the amount of the messenger RNA (mRNA) produced, i.e., the level of gene expression, also increases in the proportion of the number of copies made of the particular gene expressed.

A “tyrosine kinase inhibitor” is a molecule which inhibits to some extent tyrosine kinase activity of a tyrosine kinase such as a c-met receptor.

A cancer or biological sample which “displays c-met and/or EGFR expression, amplification, or activation” is one which, in a diagnostic test, expresses (including overexpresses) c-met and/or EGFR, has amplified c-met and/or EGFR gene, and/or otherwise demonstrates activation or phosphorylation of a c-met and/or EGFR.

A cancer or biological sample which “does not display c-met and/or EGFR expression, amplification, or activation” is one which, in a diagnostic test, does not express (including overexpress) c-met and/or EGFR, does not have amplified c-met and/or EGFR gene, and/or otherwise does not demonstrate activation or phosphorylation of a c-met and/or EGFR.

A cancer or biological sample which “displays c-met and/or EGFR activation” is one which, in a diagnostic test, demonstrates activation or phosphorylation of c-met and/or EGFR. Such activation can be determined directly (e.g. by measuring c-met and/or EGFR phosphorylation by ELISA) or indirectly.

A cancer or biological sample which “does not display c-met and/or EGFR activation” is one which, in a diagnostic test, does not demonstrate activation or phosphorylation of a c-met and/or EGFR. Such activation can be determined directly (e.g. by measuring c-met and/or EGFR phosphorylation by ELISA) or indirectly.

A cancer or biological sample which “displays c-met and/or EGFR amplification” is one which, in a diagnostic test, has amplified c-met and/or EGFR gene.

A cancer or biological sample which “does not display c-met and/or EGFR amplification” is one which, in a diagnostic test, does not have amplified c-met and/or EGFR gene.

A “phospho-ELISA assay” herein is an assay in which phosphorylation of one or more c-met and/or EGFR is evaluated in an enzyme-linked immunosorbent assay (ELISA) using a reagent, usually an antibody, to detect phosphorylated c-met and/or EGFR, substrate, or downstream signaling molecule. Preferably, an antibody which detects phosphorylated c-met and/or EGFR is used. The assay may be performed on cell lysates, preferably from fresh or frozen biological samples.

A cancer cell with “c-met and/or EGFR overexpression or amplification” is one which has significantly higher levels of a c-met and/or EGFR protein or gene compared to a noncancerous cell of the same tissue type. Such overexpression may be caused by gene amplification or by increased transcription or translation. c-met and/or EGFR overexpression or amplification may be determined in a diagnostic or prognostic assay by evaluating increased levels of the c-met and/or EGFR protein present on the surface of a cell (e.g. via an immunohistochemistry assay; IHC). Alternatively, or additionally, one may measure levels of c-met and/or EGFR-encoding nucleic acid in the cell, e.g. via fluorescent in situ hybridization (FISH; see WO98/45479 published October, 1998), southern blotting, or polymerase chain reaction (PCR) techniques, such as quantitative real time PCR (qRT-PCR). Aside from the above assays, various in vivo assays are available to the skilled practitioner. For example, one may expose cells within the body of the patient to an antibody which is optionally labeled with a detectable label, e.g. a radioactive isotope, and binding of the antibody to cells in the patient can be evaluated, e.g. by external scanning for radioactivity or by analyzing a biopsy taken from a patient previously exposed to the antibody.

A cancer cell which “does not overexpress or amplify c-met and/or EGFR” is one which does not have higher than normal levels of c-met and/or EGFR protein or gene compared to a noncancerous cell of the same tissue type.

The term “mutation”, as used herein, means a difference in the amino acid or nucleic acid sequence of a particular protein or nucleic acid (gene, RNA) relative to the wild-type protein or nucleic acid, respectively. A mutated protein or nucleic acid can be expressed from or found on one allele (heterozygous) or both alleles (homozygous) of a gene, and may be somatic or germ line. In the instant invention, mutations are generally somatic. Mutations include sequence rearrangements such as insertions, deletions, and point mutations (including single nucleotide/amino acid polymorphisms).

Protein “expression” refers to conversion of the information encoded in a gene into messenger RNA (mRNA) and then to the protein.

Herein, a sample or cell that “expresses” a protein of interest (such as a HER receptor or HER ligand) is one in which mRNA encoding the protein, or the protein, including fragments thereof, is determined to be present in the sample or cell.

The term “interleukin 8” or “IL8” or “IL-8”, as used herein, refers, unless indicated otherwise, to any native or variant (whether native or synthetic) IL8 polypeptide that is capable of activating the IL8 signaling pathway under conditions that permit such process to occur. The term “wild type IL8” generally refers to a polypeptide comprising the amino acid sequence of a naturally occurring IL8 protein. The term “wild type IL8 sequence” generally refers to an amino acid sequence found in a naturally occurring IL8.

The term “VEGF” or “VEGF-A” is used to refer to the 165-amino acid human vascular endothelial cell growth factor and related 121-, 189-, and 206-amino acid human vascular endothelial cell growth factors, as described by Leung et al. Science, 246:1306 (1989), and Houck et al. Mol. Endocrin., 5:1806 (1991), together with the naturally occurring allelic and processed forms thereof VEGF-A is part of a gene family including VEGF-B, VEGF-C, VEGF-D, VEGF-E, VEGF-F, and P1GF. VEGF-A primarily binds to two high affinity receptor tyrosine kinases, VEGFR-1 (Flt-1) and VEGFR-2 (Flk-1/KDR), the latter being the major transmitter of vascular endothelial cell mitogenic signals of VEGF-A. Additionally, neuropilin-1 has been identified as a receptor for heparin-binding VEGF-A isoforms, and may play a role in vascular development. The term “VEGF” or “VEGF-A” also refers to VEGFs from non-human species such as mouse, rat, or primate. Sometimes the VEGF from a specific species is indicated by terms such as hVEGF for human VEGF or mVEGF for murine VEGF. The term “VEGF” is also used to refer to truncated forms or fragments of the polypeptide comprising amino acids 8 to 109 or 1 to 109 of the 165-amino acid human vascular endothelial cell growth factor. Reference to any such forms of VEGF may be identified in the present application, e.g., by “VEGF (8-109),” “VEGF (1-109)” or “VEGF₁₆₅.” The amino acid positions for a “truncated” native VEGF are numbered as indicated in the native VEGF sequence. For example, amino acid position 17 (methionine) in truncated native VEGF is also position 17 (methionine) in native VEGF. The truncated native VEGF has binding affinity for the KDR and Flt-1 receptors comparable to native VEGF.

The term “VEGF variant” as used herein refers to a VEGF polypeptide which includes one or more amino acid mutations in the native VEGF sequence. Optionally, the one or more amino acid mutations include amino acid substitution(s). For purposes of shorthand designation of VEGF variants described herein, it is noted that numbers refer to the amino acid residue position along the amino acid sequence of the putative native VEGF (provided in Leung et al., supra and Houck et al., supra.).

“VEGF biological activity” includes binding to any VEGF receptor or any VEGF signaling activity such as regulation of both normal and abnormal angiogenesis and vasculogenesis (Ferrara and Davis-Smyth (1997) Endocrine Rev. 18:4-25; Ferrara (1999) J. Mol. Med. 77:527-543); promoting embryonic vasculogenesis and angiogenesis (Carmeliet et al. (1996) Nature 380:435-439; Ferrara et al. (1996) Nature 380:439-442); and modulating the cyclical blood vessel proliferation in the female reproductive tract and for bone growth and cartilage formation (Ferrara et al. (1998) Nature Med. 4:336-340; Gerber et al. (1999) Nature Med. 5:623-628). In addition to being an angiogenic factor in angiogenesis and vasculogenesis, VEGF, as a pleiotropic growth factor, exhibits multiple biological effects in other physiological processes, such as endothelial cell survival, vessel permeability and vasodilation, monocyte chemotaxis and calcium influx (Ferrara and Davis-Smyth (1997), supra and Cebe-Suarez et al. Cell. Mol. Life. Sci. 63:601-615 (2006)). Moreover, recent studies have reported mitogenic effects of VEGF on a few non-endothelial cell types, such as retinal pigment epithelial cells, pancreatic duct cells, and Schwann cells. Guerrin et al. (1995) J. Cell Physiol. 164:385-394; Oberg-Welsh et al. (1997) Mol. Cell. Endocrinol. 126:125-132; Sondell et al. (1999) J. Neurosci. 19:5731-5740.

An “angiogenesis inhibitor” or “anti-angiogenesis agent” refers to a small molecular weight substance, a polynucleotide, a polypeptide, an isolated protein, a recombinant protein, an antibody, or conjugates or fusion proteins thereof, that inhibits angiogenesis, vasculogenesis, or undesirable vascular permeability, either directly or indirectly. It should be understood that the anti-angiogenesis agent includes those agents that bind and block the angiogenic activity of the angiogenic factor or its receptor. For example, an anti-angiogenesis agent is an antibody or other antagonist to an angiogenic agent as defined above, e.g., antibodies to VEGF-A or to the VEGF-A receptor (e.g., KDR receptor or Flt-1 receptor), anti-PDGFR inhibitors such as GLEEVEC® (Imatinib Mesylate). Anti-angiogensis agents also include native angiogenesis inhibitors, e.g., angiostatin, endostatin, etc. See, e.g., Klagsbrun and D'Amore, Annu. Rev. Physiol., 53:217-39 (1991); Streit and Detmar, Oncogene, 22:3172-3179 (2003) (e.g., Table 3 listing anti-angiogenic therapy in malignant melanoma); Ferrara & Alitalo, Nature Medicine 5:1359-1364 (1999); Tonini et al., Oncogene, 22:6549-6556 (2003) (e.g., Table 2 listing known antiangiogenic factors); and Sato. Int. J. Clin. Oncol., 8:200-206 (2003) (e.g., Table 1 lists anti-angiogenic agents used in clinical trials.

A “VEGF antagonist” refers to a molecule (peptidyl or non-peptidyl) capable of neutralizing, blocking, inhibiting, abrogating, reducing, or interfering with VEGF activities including its binding to one or more VEGF receptors. In certain embodiments, the VEGF antagonist reduces or inhibits, by at least 10%, 20%, 30%, 40%, 50%, 60%, 70%, 80%, 90% or more, the expression level or biological activity of VEGF. In one embodiment, the VEGF inhibited by the VEGF antagonist is VEGF (8-109), VEGF (1-109), or VEGF₁₆₅. VEGF antagonists useful in the methods of the invention include peptidyl or non-peptidyl compounds that specifically bind VEGF, such as anti-VEGF antibodies and antigen-binding fragments thereof, polypeptides, or fragments thereof that specifically bind to VEGF, and receptor molecules and derivatives that bind specifically to VEGF thereby sequestering its binding to one or more receptors (e.g., soluble VEGF receptor proteins, or VEGF binding fragments thereof, or chimeric VEGF receptor proteins); antisense nucleobase oligomers complementary to at least a fragment of a nucleic acid molecule encoding a VEGF polypeptide; small RNAs complementary to at least a fragment of a nucleic acid molecule encoding a VEGF polypeptide; ribozymes that target VEGF; peptibodies to VEGF; and VEGF aptamers.

An “anti-VEGF antibody” is an antibody that binds to VEGF with sufficient affinity and specificity. The antibody selected will normally have a sufficiently strong binding affinity for VEGF, for example, the antibody may bind hVEGF with a K_{d} value of between 100 nM-1 pM. Antibody affinities may be determined by a surface plasmon resonance based assay (such as the BIAcore assay as described in PCT Application Publication No. WO2005/012359); enzyme-linked immunoabsorbent assay (ELISA); and competition assays (e.g. RIA's), for example. In certain embodiments, the anti-VEGF antibody of the invention can be used as a therapeutic agent in targeting and interfering with diseases or conditions wherein the VEGF activity is involved. Also, the antibody may be subjected to other biological activity assays, e.g., in order to evaluate its effectiveness as a therapeutic. Such assays are known in the art and depend on the target antigen and intended use for the antibody. Examples include the HUVEC inhibition assay (as described in the Examples below); tumor cell growth inhibition assays (as described in WO 89/06692, for example); antibody-dependent cellular cytotoxicity (ADCC) and complement-mediated cytotoxicity (CDC) assays (U.S. Pat. No. 5,500,362); and agonistic activity or hematopoiesis assays (see WO 95/27062). An anti-VEGF antibody will usually not bind to other VEGF homologues such as VEGF-B or VEGF-C, nor other growth factors such as P1GF, PDGF or bFGF.

In certain embodiments, anti-VEGF antibodies include a monoclonal antibody that binds to the same epitope as the monoclonal anti-VEGF antibody A4.6.1 produced by hybridoma ATCC HB 10709; a recombinant humanized anti-VEGF monoclonal antibody generated according to Presta et al. Cancer Res. 57:4593-4599 (1997). In one embodiment, the anti-VEGF antibody is “Bevacizumab (BV)”, also known as “rhuMAb VEGF” or “AVASTIN®”. It comprises mutated human IgG1 framework regions and antigen-binding complementarity-determining regions from the murine anti-hVEGF monoclonal antibody A.4.6.1 that blocks binding of human VEGF to its receptors. Approximately 93% of the amino acid sequence of Bevacizumab, including most of the framework regions, is derived from human IgG1, and about 7% of the sequence is derived from the murine antibody A4.6.1. Bevacizumab has a molecular mass of about 149,000 daltons and is glycosylated. Bevacizumab has been approved by the FDA for use in combination with chemotherapy regimens to treat metastatic colorectal cancer (CRC) and non-samll cell lung cancer (NSCLC). Hurwitz et al., N. Engl. J. Med. 350:2335-42 (2004); Sandler et al., N. Engl. J. Med. 355:2542-50 (2006). Currently, bevacizumab is being investigated in many ongoing clinical trials for treating various cancer indications. Kerbel, J. Clin. Oncol. 19:45 S-51S (2001); De Vore et al, Proc. Am. Soc. Clin. Oncol. 19:485a. (2000); Hurwitz et al., Clin. Colorectal Cancer 6:66-69 (2006); Johnson et al., Proc. Am. Soc. Clin. Oncol. 20:315a (2001); Kabbinavar et al. J. Clin. Oncol. 21:60-65 (2003); Miller et al., Breast Can. Res. Treat. 94:Suppl 1:S6 (2005).

Bevacizumab and other humanized anti-VEGF antibodies are further described in U.S. Pat. No. 6,884,879 issued Feb. 26, 2005. Additional antibodies include the G6 or B20 series antibodies (e.g., G6-31, B20-4.1), as described in PCT Publication No. WO2005/012359, PCT Publication No. WO2005/044853, and U.S. Patent Application 60/991,302, the content of these patent applications are expressly incorporated herein by reference. For additional antibodies see U.S. Pat. Nos. 7,060,269, 6,582,959, 6,703,020; 6,054,297; WO98/45332; WO 96/30046; WO94/10202; EP 0666868B1; U.S. Patent Application Publication Nos. 2006009360, 20050186208, 20030206899, 20030190317, 20030203409, and 20050112126; and Popkov et al., Journal of Immunological Methods 288:149-164 (2004). Other antibodies include those that bind to a functional epitope on human VEGF comprising of residues F17, M18, D19, Y21, Y25, Q89, I91, K101, E103, and C104 or, alternatively, comprising residues F17, Y21, Q22, Y25, D63, I83 and Q89.

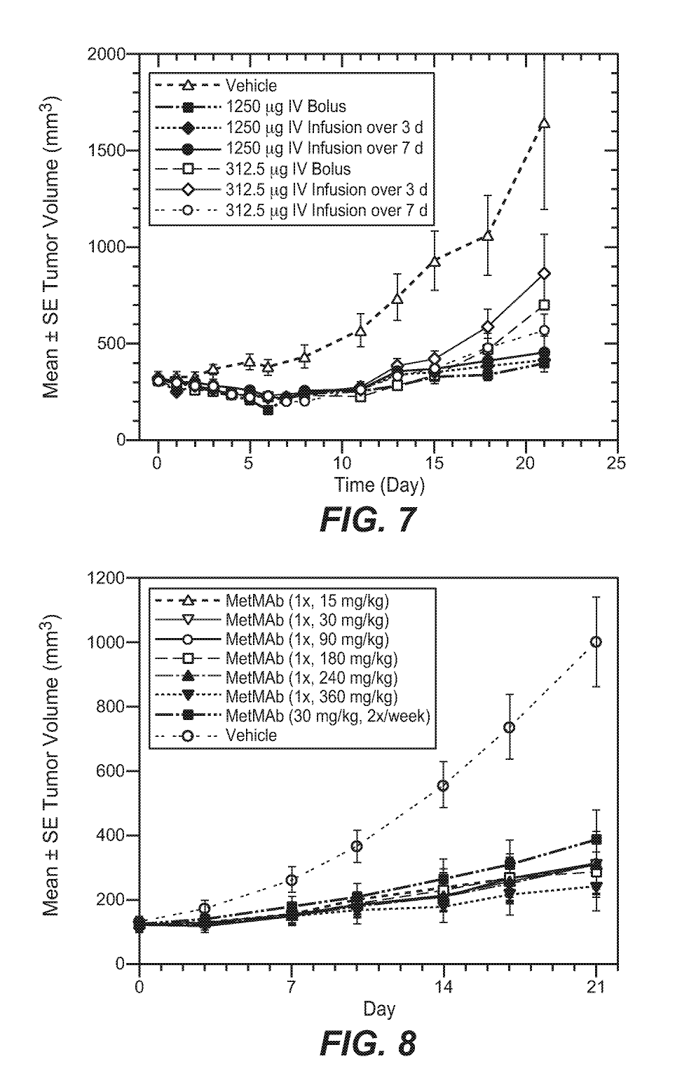

A “G6 series antibody” according to this invention, is an anti-VEGF antibody that is derived from a sequence of a G6 antibody or G6-derived antibody according to any one of FIGS. 7, 24-26, and 34-35 of PCT Publication No. WO2005/012359, the entire disclosure of which is expressly incorporated herein by reference. See also PCT Publication No. WO2005/044853, the entire disclosure of which is expressly incorporated herein by reference. In one embodiment, the G6 series antibody binds to a functional epitope on human VEGF comprising residues F17, Y21, Q22, Y25, D63, I83 and Q89.

A “B20 series antibody” according to this invention is an anti-VEGF antibody that is derived from a sequence of the B20 antibody or a B20-derived antibody according to any one of FIGS. 27-29 of PCT Publication No. WO2005/012359, the entire disclosure of which is expressly incorporated herein by reference. See also PCT Publication No. WO2005/044853, and U.S. Patent Application 60/991,302, the content of these patent applications are expressly incorporated herein by reference. In one embodiment, the B20 series antibody binds to a functional epitope on human VEGF comprising residues F17, M18, D19, Y21, Y25, Q89, I91, K101, E103, and C104.

A “functional epitope” according to this invention refers to amino acid residues of an antigen that contribute energetically to the binding of an antibody. Mutation of any one of the energetically contributing residues of the antigen (for example, mutation of wild-type VEGF by alanine or homolog mutation) will disrupt the binding of the antibody such that the relative affinity ratio (IC50mutant VEGF/IC50wild-type VEGF) of the antibody will be greater than 5 (see Example 2 of WO2005/012359). In one embodiment, the relative affinity ratio is determined by a solution binding phage displaying ELISA. Briefly, 96-well Maxisorp immunoplates (NUNC) are coated overnight at 4° C. with an Fab form of the antibody to be tested at a concentration of 2 ug/ml in PBS, and blocked with PBS, 0.5% BSA, and 0.05% Tween20 (PBT) for 2 h at room temperature. Serial dilutions of phage displaying hVEGF alanine point mutants (residues 8-109 form) or wild type hVEGF (8-109) in PBT are first incubated on the Fab-coated plates for 15 min at room temperature, and the plates are washed with PBS, 0.05% Tween20 (PBST). The bound phage is detected with an anti-M13 monoclonal antibody horseradish peroxidase (Amersham Pharmacia) conjugate diluted 1:5000 in PBT, developed with 3,3′,5,5′-tetramethylbenzidine (TMB, Kirkegaard & Perry Labs, Gaithersburg, Md.) substrate for approximately 5 min, quenched with 1.0 M H3PO4, and read spectrophotometrically at 450 nm. The ratio of IC50 values (IC50, ala/IC50, wt) represents the fold of reduction in binding affinity (the relative binding affinity).

An “immunoconjugate” (interchangeably referred to as “antibody-drug conjugate,” or “ADC”) means an antibody conjugated to one or more cytotoxic agents, such as a chemotherapeutic agent, a drug, a growth inhibitory agent, a toxin (e.g., a protein toxin, an enzymatically active toxin of bacterial, fungal, plant, or animal origin, or fragments thereof), or a radioactive isotope (i.e., a radioconjugate).

Throughout the present specification and claims, the numbering of the residues in an immunoglobulin heavy chain is that of the EU index as in Kabat et al., Sequences of Proteins of Immunological Interest, 5th Ed. Public Health Service, National Institutes of Health, Bethesda, Md. (1991), expressly incorporated herein by reference. The “EU index as in Kabat” refers to the residue numbering of the human IgG1 EU antibody.

The term “antibody” is used in the broadest sense and specifically covers monoclonal antibodies (including full length monoclonal antibodies), polyclonal antibodies, multispecific antibodies (e.g., bispecific antibodies), monovalent antibodies, multivalent antibodies, and antibody fragments so long as they exhibit the desired biological activity.

“Antibody fragments” comprise only a portion of an intact antibody, wherein the portion preferably retains at least one, preferably most or all, of the functions normally associated with that portion when present in an intact antibody. In one embodiment, an antibody fragment comprises an antigen binding site of the intact antibody and thus retains the ability to bind antigen. In another embodiment, an antibody fragment, for example one that comprises the Fc region, retains at least one of the biological functions normally associated with the Fc region when present in an intact antibody, such as FcRn binding, antibody half life modulation, ADCC function and complement binding. In one embodiment, an antibody fragment is a monovalent antibody that has an in vivo half life substantially similar to an intact antibody. For example, such an antibody fragment may comprise on antigen binding arm linked to an Fc sequence capable of conferring in vivo stability to the fragment. In one embodiment, an antibody of the invention is a one-armed antibody as described in WO2005/063816. In one embodiment, the one-armed antibody comprises Fc mutations constituting “knobs” and “holes” as described in WO2005/063816. For example, a hole mutation can be one or more of T366A, L368A and/or Y407V in an Fc polypeptide, and a cavity mutation can be T366W.

A “blocking” antibody or an antibody “antagonist” is one which inhibits or reduces biological activity of the antigen it binds. In some embodiments, blocking antibodies or antagonist antibodies completely inhibit the biological activity of the antigen.

Unless indicated otherwise, the expression “multivalent antibody” is used throughout this specification to denote an antibody comprising three or more antigen binding sites. The multivalent antibody is preferably engineered to have the three or more antigen binding sites and is generally not a native sequence IgM or IgA antibody.

An “Fv” fragment is an antibody fragment which contains a complete antigen recognition and binding site. This region consists of a dimer of one heavy and one light chain variable domain in tight association, which can be covalent in nature, for example in scFv. It is in this configuration that the three CDRs of each variable domain interact to define an antigen binding site on the surface of the V_{H}-V_{L} dimer. Collectively, the six CDRs or a subset thereof confer antigen binding specificity to the antibody. However, even a single variable domain (or half of an Fv comprising only three CDRs specific for an antigen) has the ability to recognize and bind antigen, although usually at a lower affinity than the entire binding site.

As used herein, “antibody variable domain” refers to the portions of the light and heavy chains of antibody molecules that include amino acid sequences of Complementarity Determining Regions (CDRs; ie., CDR1, CDR2, and CDR3), and Framework Regions (FRs). V_{H} refers to the variable domain of the heavy chain. V_{L} refers to the variable domain of the light chain. According to the methods used in this invention, the amino acid positions assigned to CDRs and FRs may be defined according to Kabat (Sequences of Proteins of Immunological Interest (National Institutes of Health, Bethesda, Md., 1987 and 1991)). Amino acid numbering of antibodies or antigen binding fragments is also according to that of Kabat.

As used herein, the term “Complementarity Determining Regions” (CDRs; i.e., CDR1, CDR2, and CDR3) refers to the amino acid residues of an antibody variable domain the presence of which are necessary for antigen binding. Each variable domain typically has three CDR regions identified as CDR1, CDR2 and CDR3. Each complementarity determining region may comprise amino acid residues from a “complementarity determining region” as defined by Kabat (i.e. about residues 24-34 (L1), 50-56 (L2) and 89-97 (L3) in the light chain variable domain and 31-35 (H1), 50-65 (H2) and 95-102 (H3) in the heavy chain variable domain; Kabat et al., Sequences of Proteins of Immunological Interest, 5th Ed. Public Health Service, National Institutes of Health, Bethesda, Md. (1991)) and/or those residues from a “hypervariable loop” (i.e. about residues 26-32 (L1), 50-52 (L2) and 91-96 (L3) in the light chain variable domain and 26-32 (H1), 53-55 (H2) and 96-101 (H3) in the heavy chain variable domain; Chothia and Lesk J. Mol. Biol. 196:901-917 (1987)). In some instances, a complementarity determining region can include amino acids from both a CDR region defined according to Kabat and a hypervariable loop. For example, the CDRH1 of the heavy chain of antibody 4D5 includes amino acids 26 to 35.

“Framework regions” (hereinafter FR) are those variable domain residues other than the CDR residues. Each variable domain typically has four FRs identified as FR1, FR2, FR3 and FR4. If the CDRs are defined according to Kabat, the light chain FR residues are positioned at about residues 1-23 (LCFR1), 35-49 (LCFR2), 57-88 (LCFR3), and 98-107 (LCFR4) and the heavy chain FR residues are positioned about at residues 1-30 (HCFR1), 36-49 (HCFR2), 66-94 (HCFR3), and 103-113 (HCFR4) in the heavy chain residues. If the CDRs comprise amino acid residues from hypervariable loops, the light chain FR residues are positioned about at residues 1-25 (LCFR1), 33-49 (LCFR2), 53-90 (LCFR3), and 97-107 (LCFR4) in the light chain and the heavy chain FR residues are positioned about at residues 1-25 (HCFR1), 33-52 (HCFR2), 56-95 (HCFR3), and 102-113 (HCFR4) in the heavy chain residues. In some instances, when the CDR comprises amino acids from both a CDR as defined by Kabat and those of a hypervariable loop, the FR residues will be adjusted accordingly. For example, when CDRH1 includes amino acids H26-H35, the heavy chain FR1 residues are at positions 1-25 and the FR2 residues are at positions 36-49.

The “Fab” fragment contains a variable and constant domain of the light chain and a variable domain and the first constant domain (CH1) of the heavy chain. F(ab′)₂ antibody fragments comprise a pair of Fab fragments which are generally covalently linked near their carboxy termini by hinge cysteines between them. Other chemical couplings of antibody fragments are also known in the art.

The phrase “antigen binding arm”, as used herein, refers to a component part of an antibody fragment of the invention that has an ability to specifically bind a target molecule of interest. Generally and preferably, the antigen binding arm is a complex of immunoglobulin polypeptide sequences, e.g., CDR and/or variable domain sequences of an immunoglobulin light and heavy chain.

The phrase “N-terminally truncated heavy chain”, as used herein, refers to a polypeptide comprising parts but not all of a full length immunoglobulin heavy chain, wherein the missing parts are those normally located on the N terminal region of the heavy chain. Missing parts may include, but are not limited to, the variable domain, CH1, and part or all of a hinge sequence. Generally, if the wild type hinge sequence is not present, the remaining constant domain(s) in the N-terminally truncated heavy chain would comprise a component that is capable of linkage to another Fc sequence (i.e., the “first” Fc polypeptide as described herein). For example, said component can be a modified residue or an added cysteine residue capable of forming a disulfide linkage.

The term “Fc region”, as used herein, generally refers to a dimer complex comprising the C-terminal polypeptide sequences of an immunoglobulin heavy chain, wherein a C-terminal polypeptide sequence is that which is obtainable by papain digestion of an intact antibody. The Fc region may comprise native or variant Fc sequences. Although the boundaries of the Fc sequence of an immunoglobulin heavy chain might vary, the human IgG heavy chain Fc sequence is usually defined to stretch from an amino acid residue at about position Cys226, or from about position Pro230, to the carboxyl terminus of the Fc sequence. The Fc sequence of an immunoglobulin generally comprises two constant domains, a CH2 domain and a CH3 domain, and optionally comprises a CH4 domain. By “Fc polypeptide” herein is meant one of the polypeptides that make up an Fc region. An Fc polypeptide may be obtained from any suitable immunoglobulin, such as IgG1, IgG2, IgG3, or IgG4 subtypes, IgA, IgE, IgD or IgM. In some embodiments, an Fc polypeptide comprises part or all of a wild type hinge sequence (generally at its N terminus). In some embodiments, an Fc polypeptide does not comprise a functional or wild type hinge sequence.

The terms “Fc receptor” and “FcR” are used to describe a receptor that binds to the Fc region of an antibody. For example, an FcR can be a native sequence human FcR. Generally, an FcR is one which binds an IgG antibody (a gamma receptor) and includes receptors of the FcγRI, FcγRII, and FcγRIII subclasses, including allelic variants and alternatively spliced forms of these receptors. FcγRII receptors include FcγRIIA (an “activating receptor”) and FcγRIIB (an “inhibiting receptor”), which have similar amino acid sequences that differ primarily in the cytoplasmic domains thereof. Immunoglobulins of other isotypes can also be bound by certain FcRs (see, e.g., Janeway et al., Immuno Biology: the immune system in health and disease, (Elsevier Science Ltd., NY) (4th ed., 1999)). Activating receptor FcγRIIA contains an immunoreceptor tyrosine-based activation motif (ITAM) in its cytoplasmic domain Inhibiting receptor FcγRIIB contains an immunoreceptor tyrosine-based inhibition motif (ITIM) in its cytoplasmic domain (reviewed in Daëron, Annu. Rev. Immunol. 15:203-234 (1997)). FcRs are reviewed in Ravetch and Kinet, Annu. Rev. Immunol 9:457-92 (1991); Capel et al., Immunomethods 4:25-34 (1994); and de Haas et al., J. Lab. Clin. Med. 126:330-41 (1995). Other FcRs, including those to be identified in the future, are encompassed by the term “FcR” herein. The term also includes the neonatal receptor, FcRn, which is responsible for the transfer of maternal IgGs to the fetus (Guyer et al., J. Immunol. 117:587 (1976); and Kim et al., J. Immunol. 24:249 (1994)).

The “hinge region,” “hinge sequence”, and variations thereof, as used herein, includes the meaning known in the art, which is illustrated in, for example, Janeway et al., Immuno Biology: the immune system in health and disease, (Elsevier Science Ltd., NY) (4th ed., 1999); Bloom et al., Protein Science (1997), 6:407-415; Humphreys et al., J. Immunol. Methods (1997), 209:193-202.

An “agonist antibody”, as used herein, is an antibody which mimics at least one of the functional activities of a polypeptide of interest (e.g., HGF).

“Single-chain Fv” or “scFv” antibody fragments comprise the V_{H} and V_{L} domains of antibody, wherein these domains are present in a single polypeptide chain. Generally the Fv polypeptide further comprises a polypeptide linker between the V_{H} and V_{L} domains, which enables the scFv to form the desired structure for antigen binding. For a review of scFv, see Pluckthun in The Pharmacology of Monoclonal Antibodies, Vol 113, Rosenburg and Moore eds. Springer-Verlag, New York, pp. 269-315 (1994).

The term “diabodies” refers to small antibody fragments with two antigen-binding sites, which fragments comprise a heavy chain variable domain (V_{H}) connected to a light chain variable domain (V_{L}) in the same polypeptide chain (V_{H} and V_{L}). By using a linker that is too short to allow pairing between the two domains on the same chain, the domains are forced to pair with the complementary domains of another chain and create two antigen-binding sites. Diabodies are described more fully in, for example, EP 404,097; WO 93/11161; and Hollinger et al., Proc. Natl. Acad. Sci. USA, 90:6444-6448 (1993).

The expression “linear antibodies” refers to the antibodies described in Zapata et al., Protein Eng., 8(10):1057-1062 (1995). Briefly, these antibodies comprise a pair of tandem Fd segments (V_{H}-C_{H}1-V_{H}-C_{H}1) which, together with complementary light chain polypeptides, form a pair of antigen binding regions. Linear antibodies can be bispecific or monospecific.

The modifier “monoclonal” indicates the character of the antibody as being obtained from a substantially homogeneous population of antibodies, and is not to be construed as requiring production of the antibody by any particular method. For example, the monoclonal antibodies to be used in accordance with the present invention may be made by a variety of techniques, including, for example, the hybridoma method (e.g., Kohler and Milstein, Nature, 256:495-97 (1975); Hongo et al., Hybridoma, 14 (3): 253-260 (1995), Harlow et al., Antibodies: A Laboratory Manual, (Cold Spring Harbor Laboratory Press, 2nd ed. 1988); Hammerling et al., in: Monoclonal Antibodies and T-Cell Hybridomas 563-681 (Elsevier, N.Y., 1981)), recombinant DNA methods (see, e.g., U.S. Pat. No. 4,816,567), phage-display technologies (see, e.g., Clackson et al., Nature, 352: 624-628 (1991); Marks et al., J. Mol. Biol. 222: 581-597 (1992); Sidhu et al., J. Mol. Biol. 338(2): 299-310 (2004); Lee et al., J. Mol. Biol. 340(5): 1073-1093 (2004); Fellouse, Proc. Natl. Acad. Sci. USA 101(34): 12467-12472 (2004); and Lee et al., J. Immunol. Methods 284(1-2): 119-132 (2004), and technologies for producing human or human-like antibodies in animals that have parts or all of the human immunoglobulin loci or genes encoding human immunoglobulin sequences (see, e.g., WO 1998/24893; WO 1996/34096; WO 1996/33735; WO 1991/10741; Jakobovits et al., Proc. Natl. Acad. Sci. USA 90: 2551 (1993); Jakobovits et al., Nature 362: 255-258 (1993); Bruggemann et al., Year in Immunol. 7:33 (1993); U.S. Pat. Nos. 5,545,807; 5,545,806; 5,569,825; 5,625,126; 5,633,425; and 5,661,016; Marks et al., Bio/Technology 10: 779-783 (1992); Lonberg et al., Nature 368: 856-859 (1994); Morrison, Nature 368: 812-813 (1994); Fishwild et al., Nature Biotechnol. 14: 845-851 (1996); Neuberger, Nature Biotechnol. 14: 826 (1996); and Lonberg and Huszar, Intern. Rev. Immunol. 13: 65-93 (1995).

The monoclonal antibodies herein specifically include “chimeric” antibodies in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological activity (see, e.g., U.S. Pat. No. 4,816,567; and Morrison et al., Proc. Natl. Acad. Sci. USA 81:6851-6855 (1984)). Chimeric antibodies include PRIMATIZED® antibodies wherein the antigen-binding region of the antibody is derived from an antibody produced by, e.g., immunizing macaque monkeys with the antigen of interest.

“Humanized” forms of non-human (e.g., murine) antibodies are chimeric antibodies which contain minimal sequence derived from non-human immunoglobulin. For the most part, humanized antibodies are human immunoglobulins (recipient antibody) in which residues from a hypervariable region of the recipient are replaced by residues from a hypervariable region of a non-human species (donor antibody) such as mouse, rat, rabbit or nonhuman primate having the desired specificity, affinity, and capacity. In some instances, Fv framework region (FR) residues of the human immunoglobulin are replaced by corresponding non-human residues. Furthermore, humanized antibodies may comprise residues which are not found in the recipient antibody or in the donor antibody. These modifications are made to further refine antibody performance. In general, the humanized antibody will comprise substantially all of at least one, and typically two, variable domains, in which all or substantially all of the hypervariable loops correspond to those of a non-human immunoglobulin and all or substantially all of the FR regions are those of a human immunoglobulin sequence. The humanized antibody optionally also will comprise at least a portion of an immunoglobulin constant region (Fc), typically that of a human immunoglobulin. For further details, see Jones et al., Nature 321:522-525 (1986); Riechmann et al., Nature 332:323-329 (1988); and Presta, Curr. Op. Struct. Biol. 2:593-596 (1992).

A “human antibody” is one which possesses an amino acid sequence which corresponds to that of an antibody produced by a human and/or has been made using any of the techniques for making human antibodies as disclosed herein. This definition of a human antibody specifically excludes a humanized antibody comprising non-human antigen-binding residues. Human antibodies can be produced using various techniques known in the art. In one embodiment, the human antibody is selected from a phage library, where that phage library expresses human antibodies (Vaughan et al. Nature Biotechnology 14:309-314 (1996): Sheets et al. Proc. Natl. Acad. Sci. 95:6157-6162 (1998)); Hoogenboom and Winter, J. Mol. Biol., 227:381 (1991); Marks et al., J. Mol. Biol., 222:581 (1991)). Human antibodies can also be made by introducing human immunoglobulin loci into transgenic animals, e.g., mice in which the endogenous immunoglobulin genes have been partially or completely inactivated. Upon challenge, human antibody production is observed, which closely resembles that seen in humans in all respects, including gene rearrangement, assembly, and antibody repertoire. This approach is described, for example, in U.S. Pat. Nos. 5,545,807; 5,545,806; 5,569,825; 5,625,126; 5,633,425; 5,661,016, and in the following scientific publications: Marks et al., Bio/Technology 10: 779-783 (1992); Lonberg et al., Nature 368: 856-859 (1994); Morrison, Nature 368:812-13 (1994); Fishwild et al., Nature Biotechnology 14: 845-51 (1996); Neuberger, Nature Biotechnology 14: 826 (1996); Lonberg and Huszar, Intern. Rev. Immunol. 13:65-93 (1995). Alternatively, the human antibody may be prepared via immortalization of human B lymphocytes producing an antibody directed against a target antigen (such B lymphocytes may be recovered from an individual or may have been immunized in vitro). See, e.g., Cole et al., Monoclonal Antibodies and Cancer Therapy, Alan R. Liss, p. 77 (1985); Boerner et al., J. Immunol., 147 (1):86-95 (1991); and U.S. Pat. No. 5,750,373.

A “naked antibody” is an antibody that is not conjugated to a heterologous molecule, such as a cytotoxic moiety or radiolabel.

An “affinity matured” antibody is one with one or more alterations in one or more CDRs thereof which result an improvement in the affinity of the antibody for antigen, compared to a parent antibody which does not possess those alteration(s). Preferred affinity matured antibodies will have nanomolar or even picomolar affinities for the target antigen. Affinity matured antibodies are produced by procedures known in the art. Marks et al. Bio/Technology 10:779-783 (1992) describes affinity maturation by VH and VL domain shuffling. Random mutagenesis of CDR and/or framework residues is described by: Barbas et al. Proc Nat. Acad. Sci, USA 91:3809-3813 (1994); Schier et al. Gene 169:147-155 (1995); Yelton et al. J. Immunol. 155:1994-2004 (1995); Jackson et al., J. Immunol. 154(7):3310-9 (1995); and Hawkins et al., J. Mol. Biol. 226:889-896 (1992).

An antibody having a “biological characteristic” of a designated antibody is one which possesses one or more of the biological characteristics of that antibody which distinguish it from other antibodies that bind to the same antigen.

In order to screen for antibodies which bind to an epitope on an antigen bound by an antibody of interest, a routine cross-blocking assay such as that described in Antibodies, A Laboratory Manual, Cold Spring Harbor Laboratory, Ed Harlow and David Lane (1988), can be performed.

A “functional antigen binding site” of an antibody is one which is capable of binding a target antigen. The antigen binding affinity of the antigen binding site is not necessarily as strong as the parent antibody from which the antigen binding site is derived, but the ability to bind antigen must be measurable using any one of a variety of methods known for evaluating antibody binding to an antigen. Moreover, the antigen binding affinity of each of the antigen binding sites of a multivalent antibody herein need not be quantitatively the same. For the multimeric antibodies herein, the number of functional antigen binding sites can be evaluated using ultracentrifugation analysis as described in Example 2 of U.S. Patent Application Publication No. 20050186208. According to this method of analysis, different ratios of target antigen to multimeric antibody are combined and the average molecular weight of the complexes is calculated assuming differing numbers of functional binding sites. These theoretical values are compared to the actual experimental values obtained in order to evaluate the number of functional binding sites.

A “species-dependent antibody” is one which has a stronger binding affinity for an antigen from a first mammalian species than it has for a homologue of that antigen from a second mammalian species. Normally, the species-dependent antibody “binds specifically” to a human antigen (i.e. has a binding affinity (K_{d}) value of no more than about 1×10⁻⁷ M, preferably no more than about 1×10⁻⁸ M and most preferably no more than about 1×10⁻⁹ M) but has a binding affinity for a homologue of the antigen from a second nonhuman mammalian species which is at least about 50 fold, or at least about 500 fold, or at least about 1000 fold, weaker than its binding affinity for the human antigen. The species-dependent antibody can be any of the various types of antibodies as defined above. In one embodiment, the species-dependent antibody is a humanized or human antibody.

As used herein, “antibody mutant” or “antibody variant” refers to an amino acid sequence variant of the species-dependent antibody wherein one or more of the amino acid residues of the species-dependent antibody have been modified. Such mutants necessarily have less than 100% sequence identity or similarity with the species-dependent antibody. In one embodiment, the antibody mutant will have an amino acid sequence having at least 75% amino acid sequence identity or similarity with the amino acid sequence of either the heavy or light chain variable domain of the species-dependent antibody, more preferably at least 80%, more preferably at least 85%, more preferably at least 90%, and most preferably at least 95%. Identity or similarity with respect to this sequence is defined herein as the percentage of amino acid residues in the candidate sequence that are identical (i.e same residue) or similar (i.e. amino acid residue from the same group based on common side-chain properties, see below) with the species-dependent antibody residues, after aligning the sequences and introducing gaps, if necessary, to achieve the maximum percent sequence identity. None of N-terminal, C-terminal, or internal extensions, deletions, or insertions into the antibody sequence outside of the variable domain shall be construed as affecting sequence identity or similarity.

A “chimeric VEGF receptor protein” is a VEGF receptor molecule having amino acid sequences derived from at least two different proteins, at least one of which is as VEGF receptor protein. In certain embodiments, the chimeric VEGF receptor protein is capable of binding to and inhibiting the biological activity of VEGF.

To increase the half-life of the antibodies or polypeptide containing the amino acid sequences of this invention, one can attach a salvage receptor binding epitope to the antibody (especially an antibody fragment), as described, e.g., in U.S. Pat. No. 5,739,277. For example, a nucleic acid molecule encoding the salvage receptor binding epitope can be linked in frame to a nucleic acid encoding a polypeptide sequence of this invention so that the fusion protein expressed by the engineered nucleic acid molecule comprises the salvage receptor binding epitope and a polypeptide sequence of this invention. As used herein, the term “salvage receptor binding epitope” refers to an epitope of the Fc region of an IgG molecule (e.g., IgG₁, IgG₂, IgG₃, or IgG₄) that is responsible for increasing the in vivo serum half-life of the IgG molecule (e.g., Ghetie et al., Ann. Rev. Immunol. 18:739-766 (2000), Table 1). Antibodies with substitutions in an Fc region thereof and increased serum half-lives are also described in WO00/42072, WO 02/060919; Shields et al., J. Biol. Chem. 276:6591-6604 (2001); Hinton, J. Biol. Chem. 279:6213-6216 (2004)). In another embodiment, the serum half-life can also be increased, for example, by attaching other polypeptide sequences. For example, antibodies or other polypeptides useful in the methods of the invention can be attached to serum albumin or a portion of serum albumin that binds to the FcRn receptor or a serum albumin binding peptide so that serum albumin binds to the antibody or polypeptide, e.g., such polypeptide sequences are disclosed in WO01/45746. In one preferred embodiment, the serum albumin peptide to be attached comprises an amino acid sequence of DICLPRWGCLW (SEQ ID NO:29). In another embodiment, the half-life of a Fab is increased by these methods. See also, Dennis et al. J. Biol. Chem. 277:35035-35043 (2002) for serum albumin binding peptide sequences.

An “isolated” polypeptide or “isolated” antibody is one that has been identified and separated and/or recovered from a component of its natural environment. Contaminant components of its natural environment are materials that would interfere with diagnostic or therapeutic uses for the polypeptide or antibody, and may include enzymes, hormones, and other proteinaceous or nonproteinaceous solutes. In preferred embodiments, the polypeptide or antibody will be purified (1) to greater than 95% by weight of polypeptide or antibody as determined by the Lowry method, and most preferably more than 99% by weight, (2) to a degree sufficient to obtain at least 15 residues of N-terminal or internal amino acid sequence by use of a spinning cup sequenator, or (3) to homogeneity by SDS-PAGE under reducing or nonreducing conditions using Coomassie blue or, preferably, silver stain. Isolated polypeptide or antibody includes the polypeptide or antibody in situ within recombinant cells since at least one component of the polypeptide's natural environment will not be present. Ordinarily, however, isolated polypeptide or antibody will be prepared by at least one purification step.

“Treatment” refers to both therapeutic treatment and prophylactic or preventative measures. Those in need of treatment include those already having a benign, pre-cancerous, or non-metastatic tumor as well as those in which the occurrence or recurrence of cancer is to be prevented.

The term “therapeutically effective amount” refers to an amount of a therapeutic agent to treat or prevent a disease or disorder in a mammal. In the case of cancers, the therapeutically effective amount of the therapeutic agent may reduce the number of cancer cells; reduce the primary tumor size; inhibit (i.e., slow to some extent and preferably stop) cancer cell infiltration into peripheral organs; inhibit (i.e., slow to some extent and preferably stop) tumor metastasis; inhibit, to some extent, tumor growth; and/or relieve to some extent one or more of the symptoms associated with the disorder. To the extent the drug may prevent growth and/or kill existing cancer cells, it may be cytostatic and/or cytotoxic. For cancer therapy, efficacy in vivo can, for example, be measured by assessing the duration of survival, time to disease progression (TTP), the response rates (RR), duration of response, and/or quality of life.

The terms “cancer” and “cancerous” refer to or describe the physiological condition in mammals that is typically characterized by unregulated cell growth. Included in this definition are benign and malignant cancers. By “early stage cancer” or “early stage tumor” is meant a cancer that is not invasive or metastatic or is classified as a Stage 0, I, or II cancer. Examples of cancer include, but are not limited to, carcinoma, lymphoma, blastoma (including medulloblastoma and retinoblastoma), sarcoma (including liposarcoma and synovial cell sarcoma), neuroendocrine tumors (including carcinoid tumors, gastrinoma, and islet cell cancer), mesothelioma, schwannoma (including acoustic neuroma), meningioma, adenocarcinoma, melanoma, and leukemia or lymphoid malignancies. More particular examples of such cancers include squamous cell cancer (e.g. epithelial squamous cell cancer), lung cancer including small-cell lung cancer (SCLC), non-small cell lung cancer (NSCLC), adenocarcinoma of the lung and squamous carcinoma of the lung, cancer of the peritoneum, hepatocellular cancer, gastric or stomach cancer including gastrointestinal cancer, pancreatic cancer, glioblastoma, cervical cancer, ovarian cancer, liver cancer, bladder cancer, hepatoma, breast cancer (including metastatic breast cancer), colon cancer, rectal cancer, colorectal cancer, endometrial or uterine carcinoma, salivary gland carcinoma, kidney or renal cancer, prostate cancer, vulval cancer, thyroid cancer, hepatic carcinoma, anal carcinoma, penile carcinoma, testicular cancer, esophagael cancer, tumors of the biliary tract, as well as head and neck cancer.

Herein “time to disease progression” or “TTP” refer to the time, generally measured in weeks or months, from the time of initial treatment (e.g. with a anti-cmet antibody, such as MetMAb), until the cancer progresses or worsens. Such progression can be evaluated by the skilled clinician. In the case of non-small cell lung cancer, for instance, progression can be evaluated by RECIST.

By “extending TTP” is meant increasing the time to disease progression in a treated patient relative to an untreated patient (i.e. relative to a patient not treated with a anti-cmet antibody, such as metMAb), and/or relative to a patient treated with an approved anti-tumor agent.

“Survival” refers to the patient remaining alive, and includes overall survival as well as progression free survival.

“Overall survival” refers to the patient remaining alive for a defined period of time, such as 1 year, 5 years, etc from the time of diagnosis or treatment.

“Progression free survival” refers to the patient remaining alive, without the cancer progressing or getting worse.

By “extending survival” is meant increasing overall or progression free survival in a treated patient relative to an untreated patient (i.e. relative to a patient not treated with anti-cmet antibody, such as MetMAb), and/or relative to a patient treated with an approved anti-tumor agent.

An “objective response” refers to a measurable response, including complete response (CR) or partial response (PR).

By “complete response” or “CR” is intended the disappearance of all signs of cancer in response to treatment. This does not always mean the cancer has been cured.

“Partial response” or “PR” refers to a decrease in the size of one or more tumors or lesions, or in the extent of cancer in the body, in response to treatment.

The term “pre-cancerous” refers to a condition or a growth that typically precedes or develops into a cancer. A “pre-cancerous” growth will have cells that are characterized by abnormal cell cycle regulation, proliferation, or differentiation, which can be determined by markers of cell cycle regulation, cellular proliferation, or differentiation.

By “dysplasia” is meant any abnormal growth or development of tissue, organ, or cells. Preferably, the dysplasia is high grade or precancerous.

By “metastasis” is meant the spread of cancer from its primary site to other places in the body. Cancer cells can break away from a primary tumor, penetrate into lymphatic and blood vessels, circulate through the bloodstream, and grow in a distant focus (metastasize) in normal tissues elsewhere in the body. Metastasis can be local or distant. Metastasis is a sequential process, contingent on tumor cells breaking off from the primary tumor, traveling through the bloodstream, and stopping at a distant site. At the new site, the cells establish a blood supply and can grow to form a life-threatening mass.

Both stimulatory and inhibitory molecular pathways within the tumor cell regulate this behavior, and interactions between the tumor cell and host cells in the distant site are also significant.

By “non-metastatic” is meant a cancer that is benign or that remains at the primary site and has not penetrated into the lymphatic or blood vessel system or to tissues other than the primary site. Generally, a non-metastatic cancer is any cancer that is a Stage 0, I, or II cancer, and occasionally a Stage III cancer.

By “primary tumor” or “primary cancer” is meant the original cancer and not a metastatic lesion located in another tissue, organ, or location in the subject's body.

By “benign tumor” or “benign cancer” is meant a tumor that remains localized at the site of origin and does not have the capacity to infiltrate, invade, or metastasize to a distant site.

By “tumor burden” is meant the number of cancer cells, the size of a tumor, or the amount of cancer in the body. Tumor burden is also referred to as tumor load.

By “tumor number” is meant the number of tumors.

By “subject” is meant a mammal, including, but not limited to, a human or non-human mammal, such as a bovine, equine, canine, ovine, or feline. Preferably, the subject is a human.

The term “anti-cancer therapy” refers to a therapy useful in treating cancer. Examples of anti-cancer therapeutic agents include, but are limited to, e.g., chemotherapeutic agents, growth inhibitory agents, cytotoxic agents, agents used in radiation therapy, anti-angiogenesis agents, apoptotic agents, anti-tubulin agents, and other agents to treat cancer, anti-CD20 antibodies, platelet derived growth factor inhibitors (e.g., Gleevec™ (Imatinib Mesylate)), a COX-2 inhibitor (e.g., celecoxib), interferons, cytokines, antagonists (e.g., neutralizing antibodies) that bind to one or more of the following targets ErbB2, ErbB3, ErbB4, PDGFR-beta, BlyS, APRIL, BCMA or VEGF receptor(s), TRAIL/Apo2, and other bioactive and organic chemical agents, etc. Combinations thereof are also included in the invention.

The term “cytotoxic agent” as used herein refers to a substance that inhibits or prevents the function of cells and/or causes destruction of cells. The term is intended to include radioactive isotopes (e.g., I¹³¹, I¹²⁵, Y⁹⁰ and Re¹⁸⁶), chemotherapeutic agents, and toxins such as enzymatically active toxins of bacterial, fungal, plant or animal origin, or fragments thereof.

A “chemotherapeutic agent” is a chemical compound useful in the treatment of cancer. Examples of chemotherapeutic agents include is a chemical compound useful in the treatment of cancer. Examples of chemotherapeutic agents include alkylating agents such as thiotepa and CYTOXAN® cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethiylenethiophosphoramide and trimethylolomelamine; acetogenins (especially bullatacin and bullatacinone); a camptothecin (including the synthetic analogue topotecan); bryostatin; callystatin; CC-1065 (including its adozelesin, carzelesin and bizelesin synthetic analogues); cryptophycins (particularly cryptophycin 1 and cryptophycin 8); dolastatin; duocarmycin (including the synthetic analogues, KW-2189 and CB1-TM1); eleutherobin; pancratistatin; a sarcodictyin; spongistatin; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, and ranimnustine; antibiotics such as the enediyne antibiotics (e.g., calicheamicin, especially calicheamicin gamma1I and calicheamicin omegaI1 (see, e.g., Agnew, Chem. Intl. Ed. Engl., 33: 183-186 (1994)); dynemicin, including dynemicin A; bisphosphonates, such as clodronate; an esperamicin; as well as neocarzinostatin chromophore and related chromoprotein enediyne antiobiotic chromophores), aclacinomysins, actinomycin, authramycin, azaserine, bleomycins, cactinomycin, carabicin, caminomycin, carzinophilin, chromomycinis, dactinomycin, daunorubicin, detorubicin, 6-diazo-5-oxo-L-norleucine, ADRIAMYCIN® doxorubicin (including morpholino-doxorubicin, cyanomorpholino-doxorubicin, 2-pyrrolino-doxorubicin and deoxydoxorubicin), epirubicin, esorubicin, idarubicin, marcellomycin, mitomycins such as mitomycin C, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elformithine; elliptinium acetate; an epothilone; etoglucid; gallium nitrate; hydroxyurea; lentinan; lonidainine; maytansinoids such as maytansine and ansamitocins; mitoguazone; mitoxantrone; mopidanmol; nitraerine; pentostatin; phenamet; pirarubicin; losoxantrone; podophyllinic acid; 2-ethylhydrazide; procarbazine; PSK® polysaccharide complex (JHS Natural Products, Eugene, Oreg.); razoxane; rhizoxin; sizofuran; spirogermanium; tenuazonic acid; triaziquone; 2,2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; thiotepa; taxoids, e.g., TAXOL® paclitaxel (Bristol-Myers Squibb Oncology, Princeton, N.J.), ABRAXANE™ Cremophor-free, albumin-engineered nanoparticle formulation of paclitaxel (American Pharmaceutical Partners, Schaumberg, Ill.), and TAXOTERE® doxetaxel (Rhone-Poulenc Rorer, Antony, France); chloranbucil; GEMZAR® gemcitabine; 6-thioguanine; mercaptopurine; methotrexate; platinum analogs such as cisplatin and carboplatin; vinblastine; platinum; etoposide (VP-16); ifosfamide; mitoxantrone; vincristine; NAVELBINE® vinorelbine; novantrone; teniposide; edatrexate; daunomycin; aminopterin; xeloda; ibandronate; irinotecan (Camptosar, CPT-11) (including the treatment regimen of irinotecan with 5-FU and leucovorin); topoisomerase inhibitor RFS 2000; difluoromethylornithine (DMFO); retinoids such as retinoic acid; capecitabine; combretastatin; leucovorin (LV); oxaliplatin, including the oxaliplatin treatment regimen (FOLFOX); inhibitors of PKC-alpha, Raf, H-Ras, EGFR (e.g., erlotinib (Tarceva™)) and VEGF-A that reduce cell proliferation and pharmaceutically acceptable salts, acids or derivatives of any of the above.

Also included in this definition are anti-hormonal agents that act to regulate or inhibit hormone action on tumors such as anti-estrogens and selective estrogen receptor modulators (SERMs), including, for example, tamoxifen (including NOLVADEX® tamoxifen), raloxifene, droloxifene, 4-hydroxytamoxifen, trioxifene, keoxifene, LY117018, onapristone, and FARESTON•toremifene; aromatase inhibitors that inhibit the enzyme aromatase, which regulates estrogen production in the adrenal glands, such as, for example, 4(5)-imidazoles, aminoglutethimide, MEGASE® megestrol acetate, AROMASIN® exemestane, formestanie, fadrozole, RIVISOR® vorozole, FEMARA® letrozole, and ARIMIDEX® anastrozole; and anti-androgens such as flutamide, nilutamide, bicalutamide, leuprolide, and goserelin; as well as troxacitabine (a 1,3-dioxolane nucleoside cytosine analog); antisense oligonucleotides, particularly those which inhibit expression of genes in signaling pathways implicated in abherant cell proliferation, such as, for example, PKC-alpha, Raf and H-Ras; ribozymes such as a VEGF expression inhibitor (e.g., ANGIOZYME® ribozyme) and a HER2 expression inhibitor; vaccines such as gene therapy vaccines, for example, ALLOVECTIN® vaccine, LEUVECTIN® vaccine, and VAXID® vaccine; PROLEUKIN® rIL-2; LURTOTECAN® topoisomerase 1 inhibitor; ABARELIX® rmRH; Vinorelbine and Esperamicins (see U.S. Pat. No. 4,675,187), and pharmaceutically acceptable salts, acids or derivatives of any of the above.

The term “prodrug” as used in this application refers to a precursor or derivative form of a pharmaceutically active substance that is less cytotoxic to tumor cells compared to the parent drug and is capable of being enzymatically activated or converted into the more active parent form. See, e.g., Wilman, “Prodrugs in Cancer Chemotherapy” Biochemical Society Transactions, 14, pp. 375-382, 615th Meeting Belfast (1986) and Stella et al., “Prodrugs: A Chemical Approach to Targeted Drug Delivery,” Directed Drug Delivery, Borchardt et al., (ed.), pp. 247-267, Humana Press (1985). The prodrugs of this invention include, but are not limited to, phosphate-containing prodrugs, thiophosphate-containing prodrugs, sulfate-containing prodrugs, peptide-containing prodrugs, D-amino acid-modified prodrugs, glycosylated prodrugs, β-lactam-containing prodrugs, optionally substituted phenoxyacetamide-containing prodrugs or optionally substituted phenylacetamide-containing prodrugs, 5-fluorocytosine and other 5-fluorouridine prodrugs which can be converted into the more active cytotoxic free drug. Examples of cytotoxic drugs that can be derivatized into a prodrug form for use in this invention include, but are not limited to, those chemotherapeutic agents described above.

By “radiation therapy” is meant the use of directed gamma rays or beta rays to induce sufficient damage to a cell so as to limit its ability to function normally or to destroy the cell altogether. It will be appreciated that there will be many ways known in the art to determine the dosage and duration of treatment. Typical treatments are given as a one time administration and typical dosages range from 10 to 200 units (Grays) per day.

By “reduce or inhibit” is meant the ability to cause an overall decrease of 20%, 30%, 40%, 50%, 60%, 70%, 75%, 80%, 85%, 90%, 95%, or greater. Reduce or inhibit can refer to the symptoms of the disorder being treated, the presence or size of metastases, or the size of the primary tumor.

### Therapeutic Agents

The present invention features the use of anti-c-met antagonist antibodies, such as MetMAb, in therapy to treat a pathological condition, such as tumor, in a subject. The present invention also features the use of anti-c-met antibodies and EGFR antagonists in combination therapy to treat a pathological condition, such as tumor, in a subject.

### C-Met Antagonist Antibodies

Anti-c-met antibodies that are useful in the methods of the invention include any antibody that binds with sufficient affinity and specificity to c-met and can reduce or inhibit one or more c-met activities. Anti-c-met antibodies can be used to modulate one or more aspects of HGF/c-met-associated effects, including but not limited to c-met activation, downstream molecular signaling (e.g., mitogen activated protein kinase (MAPK) phosphorylation), cell proliferation, cell migration, cell survival, cell morphogenesis and angiogenesis. These effects can be modulated by any biologically relevant mechanism, including disruption of ligand (e.g., HGF) binding to c-met, c-met phosphorylation and/or c-met multimerization.

The antibody selected will normally have a sufficiently strong binding affinity for c-met, for example, the antibody may bind human c-met with a Kd value of between 100 nM-1 pM. Antibody affinities may be determined by a surface plasmon resonance based assay (such as the BIAcore assay as described in PCT Application Publication No. WO2005/012359); enzyme-linked immunoabsorbent assay (ELISA); and competition assays (e.g. RIA's), for example. Preferably, the anti-c-met antibody of the invention can be used as a therapeutic agent in targeting and interfering with diseases or conditions wherein c-met/HGF activity is involved. Also, the antibody may be subjected to other biological activity assays, e.g., in order to evaluate its effectiveness as a therapeutic. Such assays are known in the art and depend on the target antigen and intended use for the antibody.

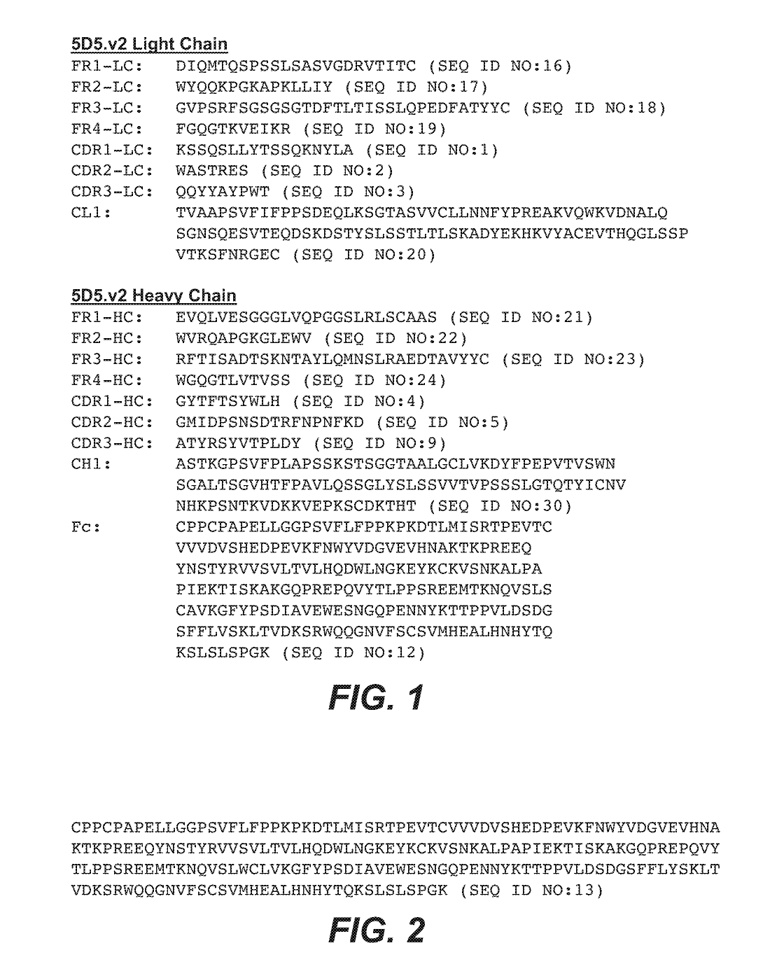

The present application discloses administration of MetMAb, a one-armed antibody comprising a Fc region, in humans for the first time. The sequence of MetMAb is shown in FIGS. 1 and 2. MetMAb (also termed OA5D5v2) is also described in, e.g., WO2006/015371; Jin et al, Cancer Res (2008) 68:4360.

Thus, the invention provides for use of anti-c-met antibodies described herein or known in the art, in the one-armed format. Accordingly, in one aspect, the anti-c-met antibody is a one-armed antibody (i.e., the heavy chain variable domain and the light chain variable domain form a single antigen binding arm) comprising an Fc region, wherein the Fc region comprises a first and a second Fc polypeptide, wherein the first and second Fc polypeptides are present in a complex and form a Fc region that increases stability of said antibody fragment compared to a Fab molecule comprising said antigen binding arm. For treatment of pathological conditions requiring an antagonistic function, and where bivalency of an antibody results in an undesirable agonistic effect, the monovalent trait of a one-armed antibody (i.e., an antibody comprising a single antigen binding arm) results in and/or ensures an antagonistic function upon binding of the antibody to a target molecule. Furthermore, the one-armed antibody comprising a Fc region is characterized by superior pharmacokinetic attributes (such as an enhanced half life and/or reduced clearance rate in vivo) compared to Fab forms having similar/substantially identical antigen binding characteristics, thus overcoming a major drawback in the use of conventional monovalent Fab antibodies. One-armed antibodies are disclosed in, for example, WO2005/063816; Martens et al, Clin Cancer Res (2006), 12: 6144.

In some embodiments, the anti-c-met antibody comprises (a) a first polypeptide comprising a heavy chain variable domain having the sequence: EVQLVESGGGLVQPGGSLRLSCAASGYTFTSYWLHWVRQAPGKGLEWVGMIDPSNSDTRFN PNFKDRFTISADTSKNTAYLQMNSLRAEDTAVYYCATYRSYVTPLDYWGQGTLVTVSS (SEQ ID NO:10), CH1 sequence, and a first Fc polypeptide; (b) a second polypeptide comprising a light chain variable domain having the sequence: DIQMTQSPSSLSASVGDRVTITCKSSQSLLYTSSQKNYLAWYQQKPGKAPKLLIYWASTR ESGVPSRFSGSGSGTDFTLTISSLQPEDFATYYCQQYYAYPWTFGQGTKVEIKR (SEQ ID NO:11), and CL1 sequence; and (c) a third polypeptide comprising a second Fc polypeptide, wherein the heavy chain variable domain and the light chain variable domain are present as a complex and form a single antigen binding arm, wherein the first and second Fc polypeptides are present in a complex and form a Fc region that increases stability of said antibody fragment compared to a Fab molecule comprising said antigen binding arm. In some embodiments, the first polypeptide comprises the Fc sequence depicted in FIG. 1 (SEQ ID NO: 12) and the second polypeptide comprises the Fc sequence depicted in FIG. 2 (SEQ ID NO: 13). In some embodiments, the first polypeptide comprises the Fc sequence depicted in FIG. 2 (SEQ ID NO: 13) and the second polypeptide comprises the Fc sequence depicted in FIG. 1 (SEQ ID NO: 12).

In some embodiments, the anti-c-met antibody comprises (a) a first polypeptide comprising a heavy chain variable domain, said polypeptide comprising the sequence: EVQLVESGGGLVQPGGSLRLSCAASGYTFTSYWLHWVRQAPGKGLEWVGMIDPSNSDTRFN PNFKDRFTISADTSKNTAYLQMNSLRAEDTAVYYCATYRSYVTPLDYWGQGTLVTVS SAST KGPSVFPLAPSSKSTSGGTAALGCLVKDYFPEPVTVSWNSGALTSGVHTFPAVLQSSGLYSLS SVVTVPSSSLGTQTYICNVNHKPSNTKVDKKVEPKSCDKTHTCPPCPAPELLGGPSVFLFPPKP KDTLMISRTPEVTCVVVDVSHEDPEVKFNWYVDGVEVHNAKTKPREEQYNSTYRVVSVLTV LHQDWLNGKEYKCKVSNKALPAPIEKTISKAKGQPREPQVYTLPPSREEMTKNQVSLSCAVK GFYPSDIAVEWESNGQPENNYKTTPPVLDSDGSFFLVSKLTVDKSRWQQGNVFSCSVMHEAL HNHYTQKSLSLSPGK (SEQ ID NO: 14); (b) a second polypeptide comprising a light chain variable domain, the polypeptide comprising the sequence DIQMTQSPSSLSASVGDRVTITCKSSQSLLYTSSQKNYLAWYQQKPGKAPKLLIYWASTRESG VPSRFSGSGSGTDFTLTISSLQPEDFATYYCQQYYAYPWTFGQGTKVEIKRTVAAPSVFIFPPS DEQLKSGTASVVCLLNNFYPREAKVQWKVDNALQSGNSQESVTEQDSKDSTYSLSSTLTLSK ADYEKHKVYACEVTHQGLSSPVTKSFNRGEC (SEQ ID NO:15); and a third polypeptide comprising a FC sequence, the polypeptide comprising the sequence CPPCPAPELLGGPSVFLFPPKPKDTLMISRTPEVTCVVVDVSHEDPEVKFNWYVDGVEVHNA KTKPREEQYNSTYRVVSVLTVLHQDWLNGKEYKCKVSNKALPAPIEKTISKAKGQPREPQV YTLPPSREEMTKNQVSLWCLVKGFYPSDIAVEWESNGQPENNYKTTPPVLDSDGSFFLYSKL TVDKSRWQQGNVFSCSVMHEALHNHYTQKSLSLSPGK (SEQ ID NO: 13), wherein the heavy chain variable domain and the light chain variable domain are present as a complex and form a single antigen binding arm, wherein the first and second Fc polypeptides are present in a complex and form a Fc region that increases stability of said antibody fragment compared to a Fab molecule comprising said antigen binding arm.

Anti-c-met antibodies (which may provided as one-armed antibodies) are known in the art (see, e.g., Martens, T, et al (2006) Clin Cancer Res 12(20 Pt 1):6144; U.S. Pat. No. 6,468,529; WO2006/015371; WO2007/063816. In one embodiment, the anti-c-met antibody comprises a heavy chain variable domain comprising one or more of CDR1-HC, CDR2-HC and CDR3-HC sequence depicted in FIG. 1 (SEQ ID NO: 4, 5, and/or 9). In some embodiments, the antibody comprises a light chain variable domain comprising one or more of CDR1-LC, CDR2-LC and CDR3-LC sequence depicted in FIG. 1 (SEQ ID NO: 1, 2, and/or 3). In some embodiments, the heavy chain variable domain comprises FR1-HC, FR2-HC, FR3-HC and FR4-HC sequence depicted in FIG. 1 (SEQ ID NO: 21-24). In some embodiments, the light chain variable domain comprises FR1-LC, FR2-LC, FR3-LC and FR4-LC sequence depicted in FIG. 1 (SEQ ID NO: 16-19).

In other embodiments, the antibody comprises one or more of the CDR sequences of the monoclonal antibody produced by the hybridoma cell line deposited under American Type Culture Collection Accession Number ATCC HB-11894 (hybridoma 1A3.3.13) or HB-11895 (hybridoma 5D5.11.6).

In one aspect, the anti-c-met antibody comprises:

(a) at least one, two, three, four or five hypervariable region (CDR) sequences selected from the group consisting of:

(i) CDR-L1 comprising sequence A1-A17, wherein A1-A17 is KSSQSLLYTSSQKNYLA (SEQ ID NO:1)

(ii) CDR-L2 comprising sequence B1-B7, wherein B1-B7 is WASTRES (SEQ ID NO:2)

(iii) CDR-L3 comprising sequence C1-C9, wherein C1-C9 is QQYYAYPWT (SEQ ID NO:3)

(iv) CDR-H1 comprising sequence D1-D10, wherein D1-D10 is GYTFTSYWLH (SEQ ID NO:4)

(v) CDR-H2 comprising sequence E1-E18, wherein E1-E18 is GMIDPSNSDTRFNPNFKD (SEQ ID NO:5) and

(vi) CDR-H3 comprising sequence F1-F11, wherein F1-F11 is XYGSYVSPLDY (SEQ ID NO:6) and X is not R;

and (b) at least one variant CDR, wherein the variant CDR sequence comprises modification of at least one residue of the sequence depicted in SEQ ID NOs:1, 2, 3, 4, 5 or 6. In one embodiment, CDR-L1 of an antibody of the invention comprises the sequence of SEQ ID NO:1. In one embodiment, CDR-L2 of an antibody of the invention comprises the sequence of SEQ ID NO:2. In one embodiment, CDR-L3 of an antibody of the invention comprises the sequence of SEQ ID NO:3. In one embodiment, CDR-H1 of an antibody of the invention comprises the sequence of SEQ ID NO:4. In one embodiment, CDR-H2 of an antibody of the invention comprises the sequence of SEQ ID NO:5. In one embodiment, CDR-H3 of an antibody of the invention comprises the sequence of SEQ ID NO:6. In one embodiment, CDR-H3 comprises TYGSYVSPLDY (SEQ ID NO: 7). In one embodiment, CDR-H3 comprises SYGSYVSPLDY (SEQ ID NO: 8). In one embodiment, an antibody of the invention comprising these sequences (in combination as described herein) is humanized or human.

In one aspect, the invention provides an antibody comprising one, two, three, four, five or six CDRs, wherein each CDR comprises, consists or consists essentially of a sequence selected from the group consisting of SEQ ID NOs: 1, 2, 3, 4, 5, 6, 7, and 8, and wherein SEQ ID NO:1 corresponds to an CDR-L1, SEQ ID NO:2 corresponds to an CDR-L2, SEQ ID NO:3 corresponds to an CDR-L3, SEQ ID NO:4 corresponds to an CDR-H1, SEQ ID NO:5 corresponds to an CDR-H2, and SEQ ID NOs:6, 7 or 8 corresponds to an CDR-H3. In one embodiment, an antibody of the invention comprises CDR-L1, CDR-L2, CDR-L3, CDR-H1, CDR-H2, and CDR-H3, wherein each, in order, comprises SEQ ID NO:1, 2, 3, 4, 5 and 7. In one embodiment, an antibody of the invention comprises CDR-L1, CDR-L2, CDR-L3, CDR-H1, CDR-H2, and CDR-H3, wherein each, in order, comprises SEQ ID NO:1, 2, 3, 4, 5 and 8.

Variant CDRs in an antibody of the invention can have modifications of one or more residues within the CDR. In one embodiment, a CDR-L2 variant comprises 1-5 (1, 2, 3, 4 or 5) substitutions in any combination of the following positions: B1 (M or L), B2 (P, T, G or S), B3 (N, G, R or T), B4 (I, N or F), B5 (P, I, L or G), B6 (A, D, T or V) and B7 (R, I, M or G). In one embodiment, a CDR-H1 variant comprises 1-5 (1, 2, 3, 4 or 5) substitutions in any combination of the following positions: D3 (N, P, L, S, A, I), D5 (I, S or Y), D6 (G, D, T, K, R), D7 (F, H, R, S, T or V) and D9 (M or V). In one embodiment, a CDR-H2 variant comprises 1-4 (1, 2, 3 or 4) substitutions in any combination of the following positions: E7 (Y), E9 (I), E10 (I), E14 (T or Q), E15 (D, K, S, T or V), E16 (L), E17 (E, H, N or D) and E18 (Y, E or H). In one embodiment, a CDR-H3 variant comprises 1-5 (1, 2, 3, 4 or 5) substitutions in any combination of the following positions: F1 (T, S), F3 (R, S, H, T, A, K), F4 (G), F6 (R, F, M, T, E, K, A, L, W), F7 (L, I, T, R, K, V), F8 (S, A), F10 (Y, N) and F11 (Q, S, H, F). Letter(s) in parenthesis following each position indicates an illustrative substitution (i.e., replacement) amino acid; as would be evident to one skilled in the art, suitability of other amino acids as substitution amino acids in the context described herein can be routinely assessed using techniques known in the art and/or described herein. In one embodiment, a CDR-L1 comprises the sequence of SEQ ID NO:1. In one embodiment, F1 in a variant CDR-H3 is T. In one embodiment, F1 in a variant CDR-H3 is S. In one embodiment, F3 in a variant CDR-H3 is R. In one embodiment, F3 in a variant CDR-H3 is S. In one embodiment, F7 in a variant CDR-H3 is T. In one embodiment, an antibody of the invention comprises a variant CDR-H3 wherein F1 is T or S, F3 is R or S, and F7 is T.

In one embodiment, an antibody of the invention comprises a variant CDR-H3 wherein F1 is T, F3 is R and F7 is T. In one embodiment, an antibody of the invention comprises a variant CDR-H3 wherein F1 is S. In one embodiment, an antibody of the invention comprises a variant CDR-H3 wherein F1 is T, and F3 is R. In one embodiment, an antibody of the invention comprises a variant CDR-H3 wherein F1 is S, F3 is R and F7 is T. In one embodiment, an antibody of the invention comprises a variant CDR-H3 wherein F1 is T, F3 is S, F7 is T, and F8 is S. In one embodiment, an antibody of the invention comprises a variant CDR-H3 wherein F1 is T, F3 is S, F7 is T, and F8 is A. In some embodiments, said variant CDR-H3 antibody further comprises CDR-L1, CDR-L2, CDR-L3, CDR-H1 and CDR-H2 wherein each comprises, in order, the sequence depicted in SEQ ID NOs:1, 2, 3, 4 and 5. In some embodiments, these antibodies further comprise a human subgroup III heavy chain framework consensus sequence. In one embodiment of these antibodies, the framework consensus sequence comprises substitution at position 71, 73 and/or 78. In some embodiments of these antibodies, position 71 is A, 73 is T and/or 78 is A. In one embodiment of these antibodies, these antibodies further comprise a human κI light chain framework consensus sequence.

In one embodiment, an antibody of the invention comprises a variant CDR-L2 wherein B6 is V. In some embodiments, said variant CDR-L2 antibody further comprises CDR-L1, CDR-L3, CDR-H1, CDR-H2 and CDR-H3, wherein each comprises, in order, the sequence depicted in SEQ ID NOs:1, 3, 4, 5 and 6. In some embodiments, said variant CDR-L2 antibody further comprises CDR-L1, CDR-L3, CDR-H1, CDR-H2 and CDR-H3, wherein each comprises, in order, the sequence depicted in SEQ ID NOs:1, 3, 4, 5 and 7. In some embodiments, said variant CDR-L2 antibody further comprises CDR-L1, CDR-L3, CDR-H1, CDR-H2 and CDR-H3, wherein each comprises, in order, the sequence depicted in SEQ ID NOs:1, 3, 4, 5 and 8. In some embodiments, these antibodies further comprise a human subgroup III heavy chain framework consensus sequence. In one embodiment of these antibodies, the framework consensus sequence comprises substitution at position 71, 73 and/or 78. In some embodiments of these antibodies, position 71 is A, 73 is T and/or 78 is A. In one embodiment of these antibodies, these antibodies further comprise a human κI light chain framework consensus sequence.

In one embodiment, an antibody of the invention comprises a variant CDR-H2 wherein E14 is T, E15 is K and E17 is E. In one embodiment, an antibody of the invention comprises a variant CDR-H2 wherein E17 is E. In some embodiments, said variant CDR-H3 antibody further comprises CDR-L1, CDR-L2, CDR-L3, CDR-H1, and CDR-H3 wherein each comprises, in order, the sequence depicted in SEQ ID NOs:1, 2, 3, 4 and 6. In some embodiments, said variant CDR-H2 antibody further comprises CDR-L1, CDR-L2, CDR-L3, CDR-H1, and CDR-H3, wherein each comprises, in order, the sequence depicted in SEQ ID NOs:1, 2, 3, 4, and 7. In some embodiments, said variant CDR-H2 antibody further comprises CDR-L1, CDR-L2, CDR-L3, CDR-H1, and CDR-H3, wherein each comprises, in order, the sequence depicted in SEQ ID NOs:1, 2, 3, 4, and 8. In some embodiments, these antibodies further comprise a human subgroup III heavy chain framework consensus sequence. In one embodiment of these antibodies, the framework consensus sequence comprises substitution at position 71, 73 and/or 78. In some embodiments of these antibodies, position 71 is A, 73 is T and/or 78 is A. In one embodiment of these antibodies, these antibodies further comprise a human κI light chain framework consensus sequence.

In other embodiments, a c-met antibody of the invention specifically binds at least a portion of c-met Sema domain or variant thereof. In one example, an antagonist antibody of the invention specifically binds at least one of the sequences selected from the group consisting of LDAQT (SEQ ID NO: 25) (e.g., residues 269-273 of c-met), LTEKRKKRS (SEQ ID NO: 26) (e.g., residues 300-308 of c-met), KPDSAEPM (SEQ ID NO: 27) (e.g., residues 350-357 of c-met) and NVRCLQHF (SEQ ID NO: 28) (e.g., residues 381-388 of c-met). In one embodiment, an antagonist antibody of the invention specifically binds a conformational epitope formed by part or all of at least one of the sequences selected from the group consisting of LDAQT (SEQ ID NO: 25) (e.g., residues 269-273 of c-met), LTEKRKKRS (SEQ ID NO: 26) (e.g., residues 300-308 of c-met), KPDSAEPM (SEQ ID NO: 27) (e.g., residues 350-357 of c-met) and NVRCLQHF (SEQ ID NO: 28) (e.g., residues 381-388 of c-met). In one embodiment, an antagonist antibody of the invention specifically binds an amino acid sequence having at least 50%, 60%, 70%, 80%, 90%, 95%, 98% sequence identity or similarity with the sequence LDAQT (SEQ ID NO: 25), LTEKRKKRS (SEQ ID NO: 26), KPDSAEPM (SEQ ID NO: 27) and/or NVRCLQHF (SEQ ID NO:28).

In one aspect, the anti-c-met antibody comprises at least one characteristic that promotes heterodimerization, while minimizing homodimerization, of the Fc sequences within the antibody fragment. Such characteristic(s) improves yield and/or purity and/or homogeneity of the immunoglobulin populations. In one embodiment, the antibody comprises Fc mutations constituting “knobs” and “holes” as described in WO2005/063816; Ridgeway, J et al, Prot Eng (1996) 9:617-21; Zhu Z et al. Prot Sci (1997) 6:781-8. For example, a hole mutation can be one or more of T366A, L368A and/or Y407V in an Fc polypeptide, and a cavity mutation can be T366W.

### EGFR Antagonists

EGFR antagonists include antibodies such as humanized monoclonal antibody known as nimotuzumab (YM Biosciences), fully human ABX-EGF (panitumumab, Abgenix Inc.) as well as fully human antibodies known as E1.1, E2.4, E2.5, E6.2, E6.4, E2.11, E6. 3 and E7.6. 3 and described in U.S. Pat. No. 6,235,883; MDX-447 (Medarex Inc). Pertuzumab (2C4) is a humanized antibody that binds directly to HER2 but interferes with HER2-EGFR dimerization thereby inhibiting EGFR signaling. Other examples of antibodies which bind to EGFR include MAb 579 (ATCC CRL HB 8506), MAb 455 (ATCC CRL HB8507), MAb 225 (ATCC CRL 8508), MAb 528 (ATCC CRL 8509) (see, U.S. Pat. No. 4,943,533, Mendelsohn et al.) and variants thereof, such as chimerized 225 (C225 or Cetuximab; ERBUTIX®) and reshaped human 225 (H225) (see, WO 96/40210, Imclone Systems Inc.); IMC-11F8, a fully human, EGFR-targeted antibody (Imclone); antibodies that bind type II mutant EGFR (U.S. Pat. No. 5,212,290); humanized and chimeric antibodies that bind EGFR as described in U.S. Pat. No. 5,891,996; and human antibodies that bind EGFR, such as ABX-EGF (see WO98/50433, Abgenix); EMD 55900 (Stragliotto et al. Eur. J. Cancer 32A:636-640 (1996)); EMD7200 (matuzumab) a humanized EGFR antibody directed against EGFR that competes with both EGF and TGF-alpha for EGFR binding; and mAb 806 or humanized mAb 806 (Johns et al., J. Biol. Chem. 279(29):30375-30384 (2004)). The anti-EGFR antibody may be conjugated with a cytotoxic agent, thus generating an immunoconjugate (see, e.g., EP659,439A2, Merck patent GmbH).

Anti-EGFR antibodies that are useful in the methods of the invention include any antibody that binds with sufficient affinity and specificity to EGFR and can reduce or inhibit EGFR activity. The antibody selected will normally have a sufficiently strong binding affinity for EGFR, for example, the antibody may bind human c-met with a Kd value of between 100 nM-1 pM. Antibody affinities may be determined by a surface plasmon resonance based assay (such as the BIAcore assay as described in PCT Application Publication No. WO2005/012359); enzyme-linked immunoabsorbent assay (ELISA); and competition assays (e.g. RIA's), for example. Preferably, the anti-c-met antibody of the invention can be used as a therapeutic agent in targeting and interfering with diseases or conditions wherein EGFR/EGFR ligand activity is involved. Also, the antibody may be subjected to other biological activity assays, e.g., in order to evaluate its effectiveness as a therapeutic. Such assays are known in the art and depend on the target antigen and intended use for the antibody.

Bispecific antibodies are antibodies that have binding specificities for at least two different epitopes. Exemplary bispecific antibodies may bind to EGFR and to c-met. In another example, an exemplary bispecific antibody may bind to two different epitopes of the same protein, e.g., c-met protein. Alternatively, a c-met or EGFR arm may be combined with an arm which binds to a triggering molecule on a leukocyte such as a T-cell receptor molecule (e.g. CD2 or CD3), or Fc receptors for IgG (FcγR), such as FcγRI (CD64), FcγRII (CD32) and FcγRIII (CD16) so as to focus cellular defense mechanisms to the c-met or EGFR-expressing cell. Bispecific antibodies may also be used to localize cytotoxic agents to cells which express EGFR or c-met. These antibodies possess a EGFR or c-met-binding arm and an arm which binds the cytotoxic agent (e.g. saporin, anti-interferon-α, vinca alkaloid, ricin A chain, methotrexate or radioactive isotope hapten). Bispecific antibodies can be prepared as full length antibodies or antibody fragments (e.g. F(ab′)₂ bispecific antibodies).

EGFR antagonists also include small molecules such as compounds described in U.S. Pat. No. 5,616,582, U.S. Pat. No. 5,457,105, U.S. Pat. No. 5,475,001, U.S. Pat. No. 5,654,307, U.S. Pat. No. 5,679,683, U.S. Pat. No. 6,084,095, U.S. Pat. No. 6,265,410, U.S. Pat. No. 6,455,534, U.S. Pat. No. 6,521,620, U.S. Pat. No. 6,596,726, U.S. Pat. No. 6,713,484, U.S. Pat. No. 5,770,599, U.S. Pat. No. 6,140,332, U.S. Pat. No. 5,866,572, U.S. Pat. No. 6,399,602, U.S. Pat. No. 6,344,459, U.S. Pat. No. 6,602,863, U.S. Pat. No. 6,391,874, WO9814451, WO9850038, WO9909016, WO9924037, WO9935146, WO0132651, U.S. Pat. No. 6,344,455, U.S. Pat. No. 5,760,041, U.S. Pat. No. 6,002,008, U.S. Pat. No. 5,747,498. Particular small molecule EGFR antagonists include OSI-774 (CP-358774, erlotinib, OSI Pharmaceuticals); PD 183805 (CI 1033, 2-propenamide, N-[4-[(3-chloro-4-fluorophenyl)amino]-7-[3-(4-morpholinyl)propoxy]-6-quinazolinyl]-, dihydrochloride, Pfizer Inc.); Iressa® (ZD1839, gefitinib, AstraZeneca); ZM 105180 ((6-amino-4-(3-methylphenyl-amino)-quinazoline, Zeneca); BIBX-1382 (N-8-(3-chloro-4-fluoro-phenyl)-N2-(1-methyl-piperidin-4-yl)-pyrimido[5,4-d]pyrimidine-2,8-diamine, Boehringer Ingelheim); PKI-166 ((R)-4-[4-[(1-phenyl ethyl)amino]-1H-pyrrolo[2,3-d]pyrimidin-6-yl]-phenol); (R)-6-(4-hydroxyphenyl)-4-[(1-phenylethyl)amino]-7H-pyrrolo[2,3-d]pyrimidine); CL-387785 (N-[4-[(3-bromophenyl)amino]-6-quinazolinyl]-2-butynamide); EKB-569 (N-[4-[(3-chloro-4-fluorophenyl)amino]-3-cyano-7-ethoxy-6-quinolinyl]-4-(dimethylamino)-2-butenamide); lapatinib (Tykerb, GlaxoSmithKline); ZD6474 (Zactima, AstraZeneca); CUDC-101 (Curis); canertinib (CI-1033); AEE788 (6-[4-[(4-ethyl-1-piperazinyl)methyl]phenyl]-N-[(1R)-1-phenylethyl]-7H-pyrrolo[2,3-d]pyrimidin-4-amine, WO2003013541, Novartis) and PKI166 4-[4-[[(1R)-1-phenylethyl]amino]-7H-pyrrolo[2,3-d]pyrimidin-6-yl]-phenol, WO9702266 Novartis).

In a particular embodiment, the EGFR antagonist has a general formula I:

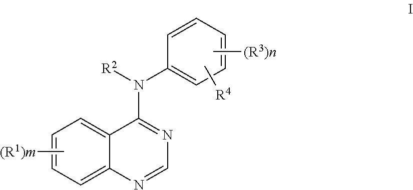

in accordance with U.S. Pat. No. 5,757,498, incorporated herein by reference, wherein:

m is 1, 2, or 3;

each R¹ is independently selected from the group consisting of hydrogen, halo, hydroxy, hydroxyamino, carboxy, nitro, guanidino, ureido, cyano, trifluoromethyl, and —(C₁-C₄ alkylene)-W-(phenyl) wherein W is a single bond, O, S or NH;

or each R¹ is independently selected from R⁹ and C₁-C₄ alkyl substituted by cyano, wherein R⁹ is selected from the group consisting of R⁵, —OR⁶, —NR⁶R⁶, —C(O)R⁷, —NHOR⁵, —OC(O)R⁶, cyano, A and —YR⁵; R⁵ is C₁-C₄ alkyl; R⁶ is independently hydrogen or R⁵; R⁷ is R⁵, —OR⁶ or —NR⁶R⁶; A is selected from piperidino, morpholino, pyrrolidino, 4-R⁶-piperazin-1-yl, imidazol-1-yl, 4-pyridon-1-yl, —(C₁-C₄ alkylene)(CO2H), phenoxy, phenyl, phenylsulfanyl, C₂-C₄ alkenyl, and —(C₁-C₄ alkylene)C(O)NR⁶R⁶; and Y is S, SO, or SO₂; wherein the alkyl moieties in R⁵, —OR⁶ and —NR⁶R⁶ are optionally substituted by one to three halo substituents and the alkyl moieties in R⁵, —OR⁶ and —NR⁶R⁶ are optionally substituted by 1 or 2 R⁹ groups, and wherein the alkyl moieties of said optional substituents are optionally substituted by halo or R⁹, with the proviso that two heteroatoms are not attached to the same carbon atom;

or each R¹ is independently selected from —NHSO₂R⁵, phthalimido-(C₁-C₄)-alkylsulfonylamino, benzamido, benzenesulfonylamino, 3-phenylureido, 2-oxopyrrolidin-1-yl, 2,5-dioxopyrrolidin-1-yl, and R¹⁰—(C₂-C₄)-alkanoylamino wherein R¹⁰ is selected from halo, —OR⁶, C₂-C₄ alkanoyloxy, —C(O)R⁷, and —NR⁶R⁶; and wherein said —NHSO₂R⁵, phthalimido-(C₁-C₄-alkylsulfonylamino, benzamido, benzenesulfonylamino, 3-phenylureido, 2-oxopyrrolidin-1-yl, 2,5-dioxopyrrolidin-1-yl, and R¹⁰—(C₂-C₄)-alkanoylamino R¹ groups are optionally substituted by 1 or 2 substituents independently selected from halo, C₁-C₄ alkyl, cyano, methanesulfonyl and C₁-C₄ alkoxy;

or two R¹ groups are taken together with the carbons to which they are attached to form a 5-8 membered ring that includes 1 or 2 heteroatoms selected from O, S and N;

R² is hydrogen or C₁-C₆ alkyl optionally substituted by 1 to 3 substituents independently selected from halo, C₁-C₄ alkoxy, —NR⁶R⁶, and —SO₂R⁵;

n is 1 or 2 and each R³ is independently selected from hydrogen, halo, hydroxy, C₁-C₆ alkyl, —NR⁶R⁶, and C₁-C₄ alkoxy, wherein the alkyl moieties of said R³ groups are optionally substituted by 1 to 3 substituents independently selected from halo, C₁-C₄ alkoxy, —NR⁶R⁶, and —SO₂R; and

R⁴ is azido or -(ethynyl)-R¹¹ wherein R¹¹ is hydrogen or C₁-C₆ alkyl optionally substituted by hydroxy, —OR⁶, or —NR⁶R⁶.

In a particular embodiment, the EGFR antagonist is a compound according to formula I selected from the group consisting of:

(6,7-dimethoxyquinazolin-4-yl)-(3-ethynylphenyl)-amine; (6,7-dimethoxyquinazolin-4-yl)-[3-(3′-hydroxypropyn-1-yl)phenyl]-amine; [3-(2′-(aminomethyl)-ethynyl)phenyl]-(6,7-dimethoxyquinazolin-4-yl)-amine; (3-ethynylphenyl)-(6-nitroquinazolin-4-yl)-amine; (6,7-dimethoxyquinazolin-4-yl)-(4-ethynylphenyl)-amine; (6,7-dimethoxyquinazolin-4-yl)-(3-ethynyl-2-methylphenyl)-amine; (6-aminoquinazolin-4-yl)-(3-ethynylphenyl)-amine; (3-ethynylphenyl)-(6-methanesulfonylaminoquinazolin-4-yl)-amine; (3-ethynylphenyl)-(6,7-methylenedioxyquinazolin-4-yl)-amine; (6,7-dimethoxyquinazolin-4-yl)-(3-ethynyl-6-methylphenyl)-amine; (3-ethynylphenyl)-(7-nitroquinazolin-4-yl)-amine; (3-ethynylphenyl)-[6-(4′-toluenesulfonylamino)quinazolin-4-yl]-amine; (3-ethynylphenyl)-{6-[2′-phthalimido-eth-1′-yl-sulfonylamino]quinazolin-4-yl}-amine; (3-ethynylphenyl)-(6-guanidinoquinazolin-4-yl)-amine; (7-aminoquinazolin-4-yl)-(3-ethynylphenyl)-amine; (3-ethynylphenyl)-(7-methoxyquinazolin-4-yl)-amine; (6-carbomethoxyquinazolin-4-yl)-(3-ethynylphenyl)-amine; (7-carbomethoxyquinazolin-4-yl)-(3-ethynylphenyl)-amine; [6,7-bis(2-methoxyethoxy)quinazolin-4-yl]-(3-ethynylphenyl)-amine; (3-azidophenyl)-(6,7-dimethoxyquinazolin-4-yl)-amine; (3-azido-5-chlorophenyl)-(6,7-dimethoxyquinazolin-4-yl)-amine; (4-azidophenyl)-(6,7-dimethoxyquinazolin-4-yl)-amine; (3-ethynylphenyl)-(6-methansulfonyl-quinazolin-4-yl)-amine; (6-ethansulfanyl-quinazolin-4-yl)-(3-ethynylphenyl)-amine; (6,7-dimethoxy-quinazolin-4-yl)-(3-ethynyl-4-fluoro-phenyl)-amine; (6,7-dimethoxy-quinazolin-4-yl)-[3-(propyn-1′-yl)-phenyl]-amine; [6,7-bis-(2-methoxy-ethoxy)-quinazolin-4-yl]-(5-ethynyl-2-methyl-phenyl)-amine; [6,7-bis-(2-methoxy-ethoxy)-quinazolin-4-yl]-(3-ethynyl-4-fluoro-phenyl)-amine; [6,7-bis-(2-chloro-ethoxy)-quinazolin-4-yl]-(3-ethynyl-phenyl)-amine; [6-(2-chloro-ethoxy)-7-(2-methoxy-ethoxy)-quinazolin-4-yl]-(3-ethynyl-phenyl)-amine; [6,7-bis-(2-acetoxy-ethoxy)-quinazolin-4-yl]-(3-ethynyl-phenyl)-amine; 2-[4-(3-ethynyl-phenylamino)-7-(2-hydroxy-ethoxy)-quinazolin-6-yloxy]-ethanol; [6-(2-acetoxy-ethoxy)-7-(2-methoxy-ethoxy)-quinazolin-4-yl]-(3-ethynyl-phenyl)-amine; [7-(2-chloro-ethoxy)-6-(2-methoxy-ethoxy)-quinazolin-4-yl]-(3-ethynyl-phenyl)-amine; [7-(2-acetoxy-ethoxy)-6-(2-methoxy-ethoxy)-quinazolin-4-yl]-(3-ethynyl-phenyl)-amine; 2-[4-(3-ethynyl-phenylamino)-6-(2-hydroxy-ethoxy)-quinazolin-7-yloxy]-ethanol; 2-[4-(3-ethynyl-phenylamino)-7-(2-methoxy-ethoxy)-quinazolin-6-yloxy]-ethanol; 2-[4-(3-ethynyl-phenylamino)-6-(2-methoxy-ethoxy)-quinazolin-7-yloxy]-ethanol; [6-(2-acetoxy-ethoxy)-7-(2-methoxy-ethoxy)-quinazolin-4-yl]-(3-ethynyl-phenyl)-amine; (3-ethynyl-phenyl)-{6-(2-methoxy-ethoxy)-7-[2-(4-methyl-piperazin-1-yl)-ethoxy]-quinazolin-4-yl}-amine; (3-ethynyl-phenyl)-[7-(2-methoxy-ethoxy)-6-(2-morpholin-4-yl)-ethoxy)-quinazolin-4-yl]-amine; (6,7-diethoxyquinazolin-1-yl)-(3-ethynylphenyl)-amine; (6,7-dibutoxyquinazolin-1-yl)-(3-ethynylphenyl)-amine; (6,7-diisopropoxyquinazolin-1-yl)-(3-ethynylphenyl)-amine; (6,7-diethoxyquinazolin-1-yl)-(3-ethynyl-2-methyl-phenyl)-amine; [6,7-bis-(2-methoxy-ethoxy)-quinazolin-1-yl]-(3-ethynyl-2-methyl-phenyl)-amine; (3-ethynylphenyl)-[6-(2-hydroxy-ethoxy)-7-(2-methoxy-ethoxy)-quinazolin-1-yl]-amine; [6,7-bis-(2-hydroxy-ethoxy)-quinazolin-1-yl]-(3-ethynylphenyl)-amine; 2-[4-(3-ethynyl-phenylamino)-6-(2-methoxy-ethoxy)-quinazolin-7-yloxy]-ethanol; (6,7-dipropoxy-quinazolin-4-yl)-(3-ethynyl-phenyl)-amine; (6,7-diethoxy-quinazolin-4-yl)-(3-ethynyl-5-fluoro-phenyl)-amine; (6,7-diethoxy-quinazolin-4-yl)-(3-ethynyl-4-fluoro-phenyl)-amine; (6,7-diethoxy-quinazolin-4-yl)-(5-ethynyl-2-methyl-phenyl)-amine; (6,7-diethoxy-quinazolin-4-yl)-(3-ethynyl-4-methyl-phenyl)-amine; (6-aminomethyl-7-methoxy-quinazolin-4-yl)-(3-ethynyl-phenyl)-amine; (6-aminomethyl-7-methoxy-quinazolin-4-yl)-(3-ethynylphenyl)-amine; (6-aminocarbonylmethyl-7-methoxy-quinazolin-4-yl)-(3-ethynylphenyl)-amine; (6-aminocarbonylethyl-7-methoxy-quinazolin-4-yl)-(3-ethynylphenyl)-amine; (6-aminocarbonylmethyl-7-ethoxy-quinazolin-4-yl)-(3-ethynylphenyl)-amine; (6-aminocarbonylethyl-7-ethoxy-quinazolin-4-yl)-(3-ethynylphenyl)-amine; (6-aminocarbonylmethyl-7-isopropoxy-quinazolin-4-yl)-(3-ethynylphenyl)-amine; (6-aminocarbonylmethyl-7-propoxy-quinazolin-4-yl)-(3-ethynylphenyl)-amine; (6-aminocarbonylmethyl-7-methoxy-quinazolin-4-yl)-(3-ethynylphenyl)-amine; (6-aminocarbonylethyl-7-isopropoxy-quinazolin-4-yl)-(3-ethynylphenyl)-amine; and (6-aminocarbonylethyl-7-propoxy-quinazolin-4-yl)-(3-ethynylphenyl)-amine; (6,7-diethoxyquinazolin-1-yl)-(3-ethynylphenyl)-amine; (3-ethynylphenyl)-[6-(2-hydroxy-ethoxy)-7-(2-methoxy-ethoxy)-quinazolin-1-yl]-amine; [6,7-bis-(2-hydroxy-ethoxy)-quinazolin-1-yl]-(3-ethynylphenyl)-amine; [6,7-bis-(2-methoxy-ethoxy)-quinazolin-1-yl]-(3-ethynylphenyl)-amine; (6,7-dimethoxyquinazolin-1-yl)-(3-ethynylphenyl)-amine; (3-ethynylphenyl)-(6-methanesulfonylamino-quinazolin-1-yl)-amine; and (6-amino-quinazolin-1-yl)-(3-ethynylphenyl)-amine

In a particular embodiment, the EGFR antagonist of formula I is N-(3-ethynylphenyl)-6,7-bis(2-methoxyethoxy)-4-quinazolinamine. In a particular embodiment, the EGFR antagonist N-(3-ethynylphenyl)-6,7-bis(2-methoxyethoxy)-4-quinazolinamine is in HCl salt form. In another particular embodiment, the EGFR antagonist N-(3-ethynylphenyl)-6,7-bis(2-methoxyethoxy)-4-quinazolinamine is in a substantially homogeneous crystalline polymorph form (described as polymorph B in WO 01/34,574) that exhibits an X-ray powder diffraction pattern having characteristic peaks expressed in degrees 2-theta at approximately 6.26, 12.48, 13.39, 16.96, 20.20, 21.10, 22.98, 24.46, 25.14 and 26.91. Such polymorph form of N-(3-ethynylphenyl)-6,7-bis(2-methoxyethoxy)-4-quinazolinamine is referred to as Tarceva™ as well as OSI-774, CP-358774 and erlotinib.

The compounds of formula I, pharmaceutically acceptable salts and prodrugs thereof (hereafter the active compounds) may be prepared by any process known to be applicable to the preparation of chemically-related compounds. In general the active compounds may be made from the appropriately substituted quinazoline using the appropriately substituted amine as shown in the general scheme I disclosed in U.S. Pat. No. 5,747,498:

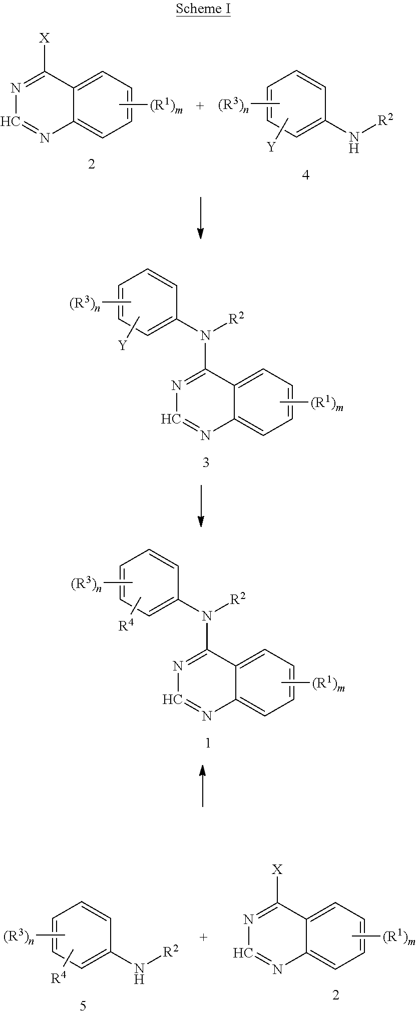

As shown in Scheme I the appropriate 4-substituted quinazoline 2 wherein X is a suitable displaceable leaving group such as halo, aryloxy, alkylsulfinyl, alkylsulfonyl such as trifluoromethanesulfonyloxy, arylsulfinyl, arylsulfonyl, siloxy, cyano, pyrazolo, triazolo or tetrazolo, preferably a 4-chloroquinazoline, is reacted with the appropriate amine or amine hydrochloride 4 or 5, wherein R⁴ is as described above and Y is Br, I, or trifluoromethane-sulfonyloxy in a solvent such as a (C₁-C₆)alcohol, dimethylformamide (DMF), N-methylpyrrolidin-2-one, chloroform, acetonitrile, tetrahydrofuran (THF), 1-4 dioxane, pyridine or other aprotic solvent. The reaction may be effected in the presence of a base, preferably an alkali or alkaline earth metal carbonate or hydroxide or a tertiary amine base, such as pyridine, 2,6-lutidine, collidine, N-methyl-morpholine, triethylamine, 4-dimethylamino-pyridine or N,N-dimethylaniline. These bases are hereinafter referred to as suitable bases. The reaction mixture is maintained at a temperature from about ambient to about the reflux temperature of the solvent, preferably from about 35° C. to about reflux, until substantially no remaining 4-haloquinazoline can be detected, typically about 2 to about 24 hours. Preferably, the reaction is performed under an inert atmosphere such as dry nitrogen.

Generally the reactants are combined stoichiometrically. When an amine base is used for those compounds where a salt (typically the HCl salt) of an amine 4 or 5 is used, it is preferable to use excess amine base, generally an extra equivalent of amine base. (Alternatively, if an amine base is not used an excess of the amine 4 or 5 may be used).

For those compounds where a sterically hindered amine 4 (such as a 2-alkyl-3-ethynylaniline) or very reactive 4-haloquinazoline is used it is preferable to use t-butyl alcohol or a polar aprotic solvent such as DMF or N-methylpyrrolidin-2-one as the solvent.

Alternatively, a 4-substituted quinazoline 2 wherein X is hydroxyl or oxo (and the 2-nitrogen is hydrogenated) is reacted with carbon tetrachloride and an optionally substituted triarylphosphine which is optionally supported on an inert polymer (e.g. triphenylphosphine, polymer supported, Aldrich Cat. No. 36,645-5, which is a 2% divinylbenzene cross-linked polystyrene containing 3 mmol phosphorous per gram resin) in a solvent such as carbon tetrachloride, chloroform, dichloroethane, tetrahydrofuran, acetonitrile or other aprotic solvent or mixtures thereof. The reaction mixture is maintained at a temperature from about ambient to reflux, preferably from about 35° C. to reflux, for 2 to 24 hours. This mixture is reacted with the appropriate amine or amine hydrochloride 4 or 5 either directly or after removal of solvent, for example by vacuum evaporation, and addition of a suitable alternative solvent such as a (C₁-C₆) alcohol, DMF, N-methylpyrrolidin-2-one, pyridine or 1-4 dioxane. Then, the reaction mixture is maintained at a temperature from about ambient to the reflux temperature of the solvent preferably from about 35° C. to about reflux, until substantially complete formation of product is achieved, typically from about 2 to about 24 hours. Preferably the reaction is performed under an inert atmosphere such as dry nitrogen.

When compound 4, wherein Y is Br, I, or trifluoromethanesulfonyloxy, is used as starting material in the reaction with quinazoline 2, a compound of formula 3 is formed wherein R¹, R², R³, and Y are as described above. Compound 3 is converted to compounds of formula I wherein R⁴ is R¹¹ ethynyl, and R¹¹ is as defined above, by reaction with a suitable palladium reagent such as tetrakis(triphenylphosphine)palladium or bis(triphenylphosphine)palladium dichloride in the presence of a suitable Lewis acid such as cuprous chloride and a suitable alkyne such as trimethylsilylacetylene, propargyl alcohol or 3-(N,N-dimethylamino)-propyne in a solvent such as diethylamine or triethylamine Compounds 3, wherein Y is NH₂, may be converted to compounds 1 wherein R⁴ is azide by treatment of compound 3 with a diazotizing agent, such as an acid and a nitrite (e.g., acetic acid and NaNO₂) followed by treatment of the resulting product with an azide, such as NaN₃.

For the production of those compounds of Formula I wherein an R¹ is an amino or hydroxyamino group the reduction of the corresponding Formula I compound wherein R¹ is nitro is employed.

The reduction may conveniently be carried out by any of the many procedures known for such transformations. The reduction may be carried out, for example, by hydrogenation of the nitro compound in a reaction-inert solvent in the presence of a suitable metal catalyst such as palladium, platinum or nickel. A further suitable reducing agent is, for example, an activated metal such as activated iron (produced by washing iron powder with a dilute solution of an acid such as hydrochloric acid). Thus, for example, the reduction may be carried out by heating a mixture of the nitro compound and the activated metal with concentrated hydrochloric acid in a solvent such as a mixture of water and an alcohol, for example, methanol or ethanol, to a temperature in the range, for example, 50° to 150° C., conveniently at or near 70° C. Another suitable class of reducing agents are the alkali metal dithionites, such as sodium dithionite, which may be used in (C₁-C₄)alkanoic acids, (C₁-C₆)alkanols, water or mixtures thereof.

For the production of those compounds of Formula I wherein R² or R³ incorporates a primary or secondary amino moiety (other than the amino group intended to react with the quinazoline), such free amino group is preferably protected prior to the above described reaction followed by deprotection, subsequent to the above described reaction with 4-(substituted)quinazoline 2.

Several well known nitrogen protecting groups can be used. Such groups include (C₁-C₆)alkoxycarbonyl, optionally substituted benzyloxycarbonyl, aryloxycarbonyl, trityl, vinyloxycarbonyl, O-nitrophenylsulfonyl, diphenylphosphinyl, p-toluenesulfonyl, and benzyl. The addition of the nitrogen protecting group may be carried out in a chlorinated hydrocarbon solvent such as methylene chloride or 1,2-dichloroethane, or an ethereal solvent such as glyme, diglyme or THF, in the presence or absence of a tertiary amine base such as triethylamine, diisopropylethylamine or pyridine, preferably triethylamine, at a temperature from about 0° C. to about 50° C., preferably about ambient temperature. Alternatively, the protecting groups are conveniently attached using Schotten-Baumann conditions.

Subsequent to the above described coupling reaction, of compounds 2 and 5, the protecting group may be removed by deprotecting methods known to those skilled in the art such as treatment with trifluoroacetic acid in methylene chloride for the tert-butoxycarbonyl protected products.

For a description of protecting groups and their use, see T. W. Greene and P. G. M. Wuts, “Protective Groups in Organic Synthesis” Second Ed., John Wiley & Sons, New York, 1991.

For the production of compounds of Formula I wherein R¹ or R² is hydroxy, cleavage of a Formula I compound wherein R¹ or R² is (C₁-C₄)alkoxy is preferred.

The cleavage reaction may conveniently be carried out by any of the many procedures known for such a transformation. Treatment of the protected formula I derivative with molten pyridine hydrochloride (20-30 eq.) at 150° to 175° C. may be employed for O-dealkylations. Alternatively, the cleavage reaction may be carried out, for example, by treatment of the protected quinazoline derivative with an alkali metal (C₁-C₄)alkylsulphide, such as sodium ethanethiolate or by treatment with an alkali metal diarylphosphide such as lithium diphenylphosphide. The cleavage reaction may also, conveniently, be carried out by treatment of the protected quinazoline derivative with a boron or aluminum trihalide such as boron tribromide. Such reactions are preferably carried out in the presence of a reaction-inert solvent at a suitable temperature.

Compounds of formula I, wherein R¹ or R² is a (C₁-C₄)alkylsulphinyl or (C₁-C₄)alkylsulphonyl group are preferably prepared by oxidation of a formula I compound wherein R¹ or R² is a (C₁-C₄)alkylsulfanyl group. Suitable oxidizing agents are known in the art for the oxidation of sulfanyl to sulphinyl and/or sulphonyl, e.g., hydrogen peroxide, a peracid (such as 3-chloroperoxybenzoic or peroxyacetic acid), an alkali metal peroxysulphate (such as potassium peroxymonosulphate), chromium trioxide or gaseous oxygen in the presence of platinum. The oxidation is generally carried out under as mild conditions as possible using the stoichiometric amount of oxidizing agent in order to reduce the risk of over oxidation and damage to other functional groups. In general, the reaction is carried out in a suitable solvent such as methylene chloride, chloroform, acetone, tetrahydrofuran or tert-butyl methyl ether and at a temperature from about −25° to 50° C., preferably at or near ambient temperature, i.e., in the range of 15° to 35° C. When a compound carrying a sulphinyl group is desired a milder oxidizing agents should be used such as sodium or potassium metaperiodate, conveniently in a polar solvent such as acetic acid or ethanol. The compounds of formula I containing a (C₁-C₄)alkylsulphonyl group may be obtained by oxidation of the corresponding (C₁-C₄)alkylsulphinyl compound as well as of the corresponding (C₁-C₄)alkylsulfanyl compound.

Compounds of formula I wherein R¹ is optionally substituted (C₂-C₄)alkanoylamino, ureido, 3-phenylureido, benzamido or sulfonamido can be prepared by acylation or sulfonylation of a corresponding compound wherein R¹ is amino Suitable acylating agents are any agents known in the art for the acylation of amino to acylamino, for example, acyl halides, e.g., a (C₂-C₄)alkanoyl chloride or bromide or a benzoyl chloride or bromide, alkanoic acid anhydrides or mixed anhydrides (e.g., acetic anhydride or the mixed anhydride formed by the reaction of an alkanoic acid and a (C₁-C₄)alkoxycarbonyl halide, for example (C₁-C₄)alkoxycarbonyl chloride, in the presence of a suitable base. For the production of those compounds of Formula I wherein R¹ is ureido or 3-phenylureido, a suitable acylating agent is, for example, a cyanate, e.g., an alkali metal cyanate such as sodium cyanate, or an isocyanate such as phenyl isocyanate. N-sulfonylations may be carried out with suitable sulfonyl halides or sulfonylanhydrides in the presence of a tertiary amine base. In general the acylation or sulfonylation is carried out in a reaction-inert solvent and at a temperature in the range of about −30° to 120° C., conveniently at or near ambient temperature.

Compounds of Formula I wherein R¹ is (C₁-C₄)alkoxy or substituted (C₁-C₄)alkoxy or R¹ is (C₁-C₄)alkylamino or substituted mono-N— or di-N,N—(C₁-C₄)alkylamino, are prepared by the alkylation, preferably in the presence of a suitable base, of a corresponding compound wherein R¹ is hydroxy or amino, respectively. Suitable alkylating agents include alkyl or substituted alkyl halides, for example, an optionally substituted (C₁-C₄)alkyl chloride, bromide or iodide, in the presence of a suitable base in a reaction-inert solvent and at a temperature in the range of about 10° to 140° C., conveniently at or near ambient temperature.

For the production of those compounds of Formula I wherein R¹ is an amino-, oxy- or cyano-substituted (C₁-C₄)alkyl substituent, a corresponding compound wherein R¹ is a (C₁-C₄)alkyl substituent bearing a group which is displacable by an amino-, alkoxy-, or cyano group is reacted with an appropriate amine, alcohol or cyanide, preferably in the presence of a suitable base. The reaction is preferably carried out in a reaction-inert solvent or diluent and at a temperature in the range of about 10° to 100° C., preferably at or near ambient temperature.

Compounds of Formula I, wherein R¹ is a carboxy substituent or a substituent which includes a carboxy group are prepared by hydrolysis of a corresponding compound wherein R¹ is a (C₁-C₄)alkoxycarbonyl substituent or a substituent which includes a (C₁-C₄)alkoxycarbonyl group. The hydrolysis may conveniently be performed, for example, under basic conditions, e.g., in the presence of alkali metal hydroxide.

Compounds of Formula I wherein R¹ is amino, (C₁-C₄)alkylamino, di-[(C₁-C₄)alkyl]amino, pyrrolidin-1-yl, piperidino, morpholino, piperazin-1-yl, 4-(C₁-C₄)alkylpiperazin-1-yl or (C₁-C₄)alkysulfanyl, may be prepared by the reaction, in the presence of a suitable base, of a corresponding compound wherein R¹ is an amine or thiol displaceable group with an appropriate amine or thiol. The reaction is preferably carried out in a reaction-inert solvent or diluent and at a temperature in the range of about 10° to 180° C., conveniently in the range 100° to 150° C.

Compounds of Formula I wherein R¹ is 2-oxopyrrolidin-1-yl or 2-oxopiperidin-1-yl are prepared by the cyclisation, in the presence of a suitable base, of a corresponding compound wherein R¹ is a halo-(C₂-C₄)alkanoylamino group. The reaction is preferably carried out in a reaction-inert solvent or diluent and at a temperature in the range of about 10° to 100° C., conveniently at or near ambient temperature.

For the production of compounds of Formula I in which R¹ is carbamoyl, substituted carbamoyl, alkanoyloxy or substituted alkanoyloxy, the carbamoylation or acylation of a corresponding compound wherein R¹ is hydroxy is convenient.

Suitable acylating agents known in the art for acylation of hydroxyaryl moieties to alkanoyloxyaryl groups include, for example, (C₂-C₄)alkanoyl halides, (C₂-C₄)alkanoyl anhydrides and mixed anhydrides as described above, and suitable substituted derivatives thereof may be employed, typically in the presence of a suitable base. Alternatively, (C₂-C₄)alkanoic acids or suitably substituted derivatives thereof may be coupled with a Formula I compound wherein R¹ is hydroxy with the aid of a condensing agent such as a carbodiimide. For the production of those compounds of Formula I in which R¹ is carbamoyl or substituted carbamoyl, suitable carbamoylating agents are, for example, cyanates or alkyl or arylisocyanates, typically in the presence of a suitable base. Alternatively, suitable intermediates such as the chloroformate or carbonylimidazolyl derivative of a compound of Formula I in which R¹ is hydroxy may be generated, for example, by treatment of said derivative with phosgene (or a phosgene equivalent) or carbonyldiimidazole. The resulting intermediate may then be reacted with an appropriate amine or substituted amine to produce the desired carbamoyl derivatives.

Compounds of formula I wherein R¹ is aminocarbonyl or a substituted aminocarbonyl can be prepared by the aminolysis of a suitable intermediate in which R¹ is carboxy.

The activation and coupling of formula I compounds wherein R¹ is carboxy may be performed by a variety of methods known to those skilled in the art. Suitable methods include activation of the carboxyl as an acid halide, azide, symmetric or mixed anhydride, or active ester of appropriate reactivity for coupling with the desired amine Examples of such types of intermediates and their production and use in couplings with amines may be found extensively in the literature; for example M. Bodansky and A. Bodansky, “The Practice of Peptide Synthesis”, Springer-Verlag, New York, 1984. The resulting formula I compounds may be isolated and purified by standard methods, such as solvent removal and recrystallization or chromatography.

The starting materials for the described reaction scheme I (e.g., amines, quinazolines and amine protecting groups) are readily available or can be easily synthesized by those skilled in the art using conventional methods of organic synthesis. For example, the preparation of 2,3-dihydro-1,4-benzoxazine derivatives are described in R. C. Elderfield, W. H. Todd, S. Gerber, Ch. 12 in “Heterocyclic Compounds”, Vol. 6, R. C. Elderfield ed., John Wiley and Sons, Inc., N.Y., 1957. Substituted 2,3-dihydrobenzothiazinyl compounds are described by R. C. Elderfield and E. E. Harris in Ch. 13 of Volume 6 of the Elderfield “Heterocyclic Compounds” book.

In another particular embodiment, the EGFR antagonist has a general formula II as described in U.S. Pat. No. 5,457,105, incorporated herein by reference:

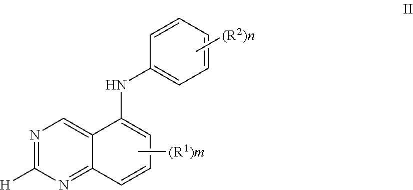

wherein:

m is 1, 2 or 3 and

each R¹ is independently 6-hydroxy, 7-hydroxy, amino, carboxy, carbamoyl, ureido, (1-4C)alkoxycarbonyl, N-(1-4C)alkylcarbamoyl, N,N-di-[(1-4C)alkyl]carbamoyl, hydroxyamino, (1-4C)alkoxyamino, (2-4C)alkanoyloxyamino, trifluoromethoxy, (1-4C)alkyl, 6-(1-4C)alkoxy, 7-(1-4C)alkoxy, (1-3C)alkylenedioxy, (1-4C)alkylamino, di-1[(1-4C)alkyl]amino, pyrrolidin-1-yl, piperidino, morpholino, piperazin-1-yl, 4-(1-4C)alkylpiperazin-1-yl, (1-4C)alkylthio, (1-4C)alkylsulphinyl, (1-4C)alkylsulphonyl, bromomethyl, dibromomethyl, hydroxy-(1-4C)alkyl, (2-4C)alkanoyloxy-(1-4C)alkyl, (1-4C)alkoxy-(1-4C)alkyl, carboxy-(1-4C)alkyl, (1-4C)alkoxycarbonyl-(1-4C)alkyl, carbamoyl-(1-4C)alkyl, N-(1-4C)alkylcarbamoyl-(1-4C)alkyl, N,N-di-[(1-4C)alkyl]carbamoyl-(1-4C)alkyl, amino-(1-4C)alkyl, (1-4C)alkylamino-(1-4C)alkyl, di-[(1-4C)alkyl]amino-(1-4C)alkyl, piperidino-(1-4C)alkyl, morpholino-(1-4C)alkyl, piperazin-1-yl-(1-4C) alkyl, 4-(1-4C)alkylpiperazin-1-yl-(1-4C) alkyl, hydroxy-(2-4C)alkoxy-(1-4C) alkyl, (1-4C)alkoxy-(2-4C)alkoxy-(1-4C)alkyl, hydroxy-(2-4C)alkylamino-(1-4C)alkyl, (1-4C)alkoxy-(2-4C)alkylamino-(1-4C)alkyl, (1-4C)alkylthio-(1-4C)alkyl, hydroxy-(2-4C)alkylthio-(1-4C)alkyl, (1-4C)alkoxy-(2-4C)alkylthio-(1-4C)alkyl, phenoxy-(1-4C)alkyl, anilino-(1-4C)alkyl, phenylthio-(1-4C)alkyl, cyano-(1-4C)alkyl, halogeno-(2-4C)alkoxy, hydroxy-(2-4C)alkoxy, (2-4C)alkanoyloxy-(2-4C)alkoxy, (1-4C)alkoxy-(2-4C)alkoxy, carboxy-(1-4C)alkoxy, (1-4C)alkoxycarbonyl-(1-4C)alkoxy, carbamoyl-(1-4C)alkoxy, N-(1-4C) alkylcarbamoyl-(1-4C)alkoxy, N,N-di-[(1-4C)alkyl]carbamoyl-(1-4C)alkoxy, amino-(2-4C)alkoxy, (1-4C)alkylamino-(2-4C)alkoxy, di-[(1-4C)alkyl]amino-(2-4C)alkoxy, (2-4C)alkanoyloxy, hydroxy-(2-4C)alkanoyloxy, (1-4C)alkoxy-(2-4C)alkanoyloxy, phenyl-(1-4C)alkoxy, phenoxy-(2-4C)alkoxy, anilino-(2-4C)alkoxy, phenylthio-(2-4C)alkoxy, piperidino-(2-4C)alkoxy, morpholino-(2-4C)alkoxy, piperazin-1-yl-(2-4C)alkoxy, 4-(1-4C)alkylpiperazin-1-yl-(2-4C)alkoxy, halogeno-(2-4C)alkylamino, hydroxy-(2-4C)alkylamino, (2-4C)alkanoyloxy-(2-4C)alkylamino, (1-4C)alkoxy-(2-4C)alkylamino, carboxy-(1-4C)alkylamino, (1-4C)alkoxycarbonyl-(1-4C)alkylamino, carbamoyl-(1-4C)alkylamino, N-(1-4C)alkylcarbamoyl-(1-4C)alkylamino, N,N-di-[(1-4C)alkyl]carbamoyl-(1-4C)alkylamino, amino-(2-4C)alkylamino, (1-4C)alkylamino-(2-4C)alkylamino, di-[(1-4C)alkyl]amino-(2-4C)alkylamino, phenyl-(1-4C)alkylamino, phenoxy-(2-4C)alkylamino, anilino-(2-4C)alkylamino, phenylthio-(2-4C)alkylamino, (2-4C)alkanoylamino, (1-4C)alkoxycarbonylamino, (1-4C)alkylsulphonylamino, benzamido, benzenesulphonamido, 3-phenylureido, 2-oxopyrrolidin-1-yl, 2,5-dioxopyrrolidin-1-yl, halogeno-(2-4C)alkanoylamino, hydroxy-(2-4C)alkanoylamino, (1-4C)alkoxy-(2-4C)alkanoylamino, carboxy-(2-4C)alkanoylamino, (1-4C)alkoxycarbonyl-(2-4C)alkanoylamino, carbamoyl-(2-4C)alkanoylamino, N-(1-4C)alkylcarbamoyl-(2-4C)alkanoylamino, N,N-di-[(1-4C)alkyl]carbamoyl-(2-4C)alkanoylamino, amino-(2-4C)alkanoylamino, (1-4C)alkylamino-(2-4C)alkanoylamino or di-[(1-4C)alkyl]amino-(2-4C)alkanoylamino, and wherein said benzamido or benzenesulphonamido substituent or any anilino, phenoxy or phenyl group in a R¹ substituent may optionally bear one or two halogeno, (1-4C)alkyl or (1-4C)alkoxy substituents;
n is 1 or 2 and

each R² is independently hydrogen, hydroxy, halogeno, trifluoromethyl, amino, nitro, cyano, (1-4C)alkyl, (1-4C)alkoxy, (1-4C)alkylamino, di-[(1-4C)alkyl]amino, (1-4C)alkylthio, (1-4C)alkylsulphinyl or (1-4C)alkylsulphonyl; or a pharmaceutically-acceptable salt thereof; except that 4-(4′-hydroxyanilino)-6-methoxyquinazoline, 4-(4,-hydroxyanilino)-6,7-methylenedioxyquinazoline, 6-amino-4-(4′-aminoanilino)quinazoline, 4-anilino-6-methylquinazoline or the hydrochloride salt thereof and 4-anilino-6,7-dimethoxyquinazoline or the hydrochloride salt thereof are excluded.

In a particular embodiment, the EGFR antagonist is a compound according to formula II selected from the group consisting of: 4-(3′-chloro-4′-fluoroanilino)-6,7-dimethoxyquinazoline; 4-(3′,4′-dichloroanilino)-6,7-dimethoxyquinazoline; 6,7-dimethoxy-4-(3′-nitroanilino)-quinazoline; 6,7-diethoxy-4-(3′-methylanilino)-quinazoline; 6-methoxy-4-(3′-methylanilino)-quinazoline; 4-(3′-chloroanilino)-6-methoxyquinazoline; 6,7-ethylenedioxy-4-(3′-methylanilino)-quinazoline; 6-amino-7-methoxy-4-(3′-methylanilino)-quinazoline; 4-(3′-methylanilino)-6-ureidoquinazoline; 6-(2-methoxyethoxymethyl)-4-(3′-methylanilino)-quinazoline; 6,7-di-(2-methoxyethoxy)-4-(3′-methylanilino)-quinazoline; 6-dimethylamino-4-(3′-methylanilino)quinazoline; 6-benzamido-4-(3′-methylanilino)quinazoline; 6,7-dimethoxy-4-(3′-trifluoromethylanilino)-quinazoline; 6-hydroxy-7-methoxy-4-(3′-methylanilino)-quinazoline; 7-hydroxy-6-methoxy-4-(3′-methylanilino)-quinazoline; 7-amino-4-(3′-methylanilino)-quinazoline; 6-amino-4-(3′-methylanilino)quinazoline; 6-amino-4-(3′-chloroanilino)-quinazoline; 6-acetamido-4-(3′-methylanilino)-quinazoline; 6-(2-methoxyethylamino)-4-(3′-methylanilino)-quinazoline; 7-(2-methoxyacetamido)-4-(3′-methylanilino)-quinazoline; 7-(2-hydroxyethoxy)-6-methoxy-4-(3′-methylanilino)-quinazoline; 7-(2-methoxyethoxy)-6-methoxy-4-(3′-methylanilino)-quinazoline; 6-amino-4-(3′-methylanilino)-quinazoline.

A quinazoline derivative of the formula II, or a pharmaceutically-acceptable salt thereof, may be prepared by any process known to be applicable to the preparation of chemically-related compounds. A suitable process is, for example, illustrated by that used in U.S. Pat. No. 4,322,420. Necessary starting materials may be commercially available or obtained by standard procedures of organic chemistry.

(a) The reaction, conveniently in the presence of a suitable base, of a quinazoline (i), wherein Z is a displaceable group, with an aniline (ii).

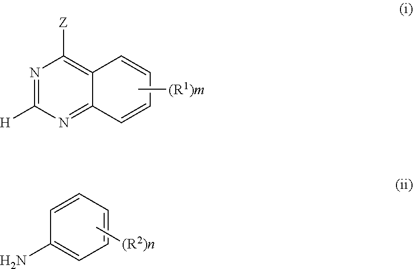

A suitable displaceable group Z is, for example, a halogeno, alkoxy, aryloxy or sulphonyloxy group, for example a chloro, bromo, methoxy, phenoxy, methanesulphonyloxy or toluene-p-sulphonyloxy group.

A suitable base is, for example, an organic amine base such as, for example, pyridine, 2,6-lutidine, collidine, 4-dimethylaminopyridine, triethylamine, morpholine, N-methylmorpholine or diazabicyclo[5.4.0]undec-7-ene, or for example, an alkali or alkaline earth metal carbonate or hydroxide, for example sodium carbonate, potassium carbonate, calcium carbonate, sodium hydroxide or potassium hydroxide.

The reaction is preferably carried out in the presence of a suitable inert solvent or diluent, for example an alkanol or ester such as methanol, ethanol, isopropanol or ethyl acetate, a halogenated solvent such as methylene chloride, chloroform or carbon tetrachloride, an ether such as tetrahydrofuran or 1,4-dioxan, an aromatic solvent such as toluene, or a dipolar aprotic solvent such as N,N-dimethylformamide, N,N-dimethylacetamide, N-methylpyrrolidin-2-one or dimethylsulphoxide. The reaction is conveniently carried out at a temperature in the range, for example, 10° to 150° C., preferably in the range 20° to 80° C.

The quinazoline derivative of the formula II may be obtained from this process in the form of the free base or alternatively it may be obtained in the form of a salt with the acid of the formula H—Z wherein Z has the meaning defined hereinbefore. When it is desired to obtain the free base from the salt, the salt may be treated with a suitable base as defined hereinbefore using a conventional procedure.

(b) For the production of those compounds of the formula II wherein R¹ or R² is hydroxy, the cleavage of a quinazoline derivative of the formula II wherein R¹ or R² is (1-4C)alkoxy.

The cleavage reaction may conveniently be carried out by any of the many procedures known for such a transformation. The reaction may be carried out, for example, by treatment of the quinazoline derivative with an alkali metal (1-4C)alkylsulphide such as sodium ethanethiolate or, for example, by treatment with an alkali metal diarylphosphide such as lithium diphenylphosphide. Alternatively the cleavage reaction may conveniently be carried out, for example, by treatment of the quinazoline derivative with a boron or aluminium trihalide such as boron tribromide. Such reactions are preferably carried out in the presence of a suitable inert solvent or diluent as defined hereinbefore and at a suitable temperature.

(c) For the production of those compounds of the formula II wherein R¹ or R² is a (1-4C)alkylsulphinyl or (1-4C)alkylsulphonyl group, the oxidation of a quinazoline derivative of the formula II wherein R¹ or R² is a (1-4C)alkylthio group.

A suitable oxidising agent is, for example, any agent known in the art for the oxidation of thio to sulphinyl and/or sulphonyl, for example, hydrogen peroxide, a peracid (such as 3-chloroperoxybenzoic or peroxyacetic acid), an alkali metal peroxysulphate (such as potassium peroxymonosulphate), chromium trioxide or gaseous oxygen in the presence of platinium. The oxidation is generally carried out under as mild conditions as possible and with the required stoichiometric amount of oxidising agent in order to reduce the risk of over oxidation and damage to other functional groups. In general the reaction is carried out in a suitable solvent or diluent such as methylene chloride, chloroform, acetone, tetrahydrofuran or tert-butyl methyl ether and at a temperature, for example, −25° to 50° C., conveniently at or near ambient temperature, that is in the range 15° to 35° C. When a compound carrying a sulphinyl group is required a milder oxidising agent may also be used, for example sodium or potassium metaperiodate, conveniently in a polar solvent such as acetic acid or ethanol. It will be appreciated that when a compound of the formula II containing a (1-4C)alkylsulphonyl group is required, it may be obtained by oxidation of the corresponding (1-4C)alkylsulphinyl compound as well as of the corresponding (1-4C)alkylthio compound.

(d) For the production of those compounds of the formula II wherein R¹ is amino, the reduction of a quinazoline derivative of the formula I wherein R¹ is nitro.

The reduction may conveniently be carried out by any of the many procedures known for such a transformation. The reduction may be carried out, for example, by the hydrogenation of a solution of the nitro compound in an inert solvent or diluent as defined hereinbefore in the presence of a suitable metal catalyst such as palladium or platinum. A further suitable reducing agent is, for example, an activated metal such as activated iron (produced by washing iron powder with a dilute solution of an acid such as hydrochloric acid). Thus, for example, the reduction may be carried out by heating a mixture of the nitro compound and the activated metal in a suitable solvent or diluent such as a mixture of water and an alcohol, for example, methanol or ethanol, to a temperature in the range, for example, 50° to 150° C., conveniently at or near 70° C.

(e) For the production of those compounds of the formula II wherein R¹ is (2-4C)alkanoylamino or substituted (2-4C)alkanoylamino, ureido, 3-phenylureido or benzamido, or R² is acetamido or benzamido, the acylation of a quinazoline derivative of the formula II wherein R¹ or R² is amino

A suitable acylating agent is, for example, any agent known in the art for the acylation of amino to acylamino, for example an acyl halide, for example a (2-4C)alkanoyl chloride or bromide or a benzoyl chloride or bromide, conveniently in the presence of a suitable base, as defined hereinbefore, an alkanoic acid anhydride or mixed anhydride, for example a (2-4C)alkanoic acid anhydride such as acetic anhydride or the mixed anhydride formed by the reaction of an alkanoic acid and a (1-4C)alkoxycarbonyl halide, for example a (1-4C)alkoxycarbonyl chloride, in the presence of a suitable base as defined hereinbefore. For the production of those compounds of the formula II wherein R¹ is ureido or 3-phenylureido, a suitable acylating agent is, for example, a cyanate, for example an alkali metal cyanate such as sodium cyanate or, for example, an isocyanate such as phenyl isocyanate. In general the acylation is carried out in a suitable inert solvent or diluent as defined hereinbefore and at a temperature, in the range, for example, −30° to 120° C., conveniently at or near ambient temperature.

(f) For the production of those compounds of the formula II wherein R¹ is (1-4C)alkoxy or substituted (1-4C)alkoxy or R¹ is (1-4C)alkylamino or substituted (1-4C)alkylamino, the alkylation, preferably in the presence of a suitable base as defined hereinbefore, of a quinazoline derivative of the formula II wherein R¹ is hydroxy or amino as appropriate.

A suitable alkylating agent is, for example, any agent known in the art for the alkylation of hydroxy to alkoxy or substituted alkoxy, or for the alkylation of amino to alkylamino or substituted alkylamino, for example an alkyl or substituted alkyl halide, for example a (1-4C)alkyl chloride, bromide or iodide or a substituted (1-4C)alkyl chloride, bromide or iodide, in the presence of a suitable base as defined hereinbefore, in a suitable inert solvent or diluent as defined hereinbefore and at a temperature in the range, for example, 10° to 140° C., conveniently at or near ambient temperature.

(g) For the production of those compounds of the formula II wherein R¹ is a carboxy substituent or a substituent which includes a carboxy group, the hydrolysis of a quinazoline derivative of the formula II wherein R¹ is a (1-4C)alkoxycarbonyl substituent or a substituent which includes a (1-4C)alkoxycarbonyl group.

The hydrolysis may conveniently be performed, for example, under basic conditions.

(h) For the production of those compounds of the formula II wherein R¹ is an amino-, oxy-, thio- or cyano-substituted (1-4C)alkyl substituent, the reaction, preferably in the presence of a suitable base as defined hereinbefore, of a quinazoline derivative of the formula II wherein R¹ is a (1-4C)alkyl substituent bearing a displaceable group as defined hereinbefore with an appropriate amine, alcohol, thiol or cyanide.

The reaction is preferably carried out in a suitable inert solvent or diluent as defined hereinbefore and at a temperature in the range, for example, 10° to 100° C., conveniently at or near ambient temperature.

When a pharmaceutically-acceptable salt of a quinazoline derivative of the formula II is required, it may be obtained, for example, by reaction of said compound with, for example, a suitable acid using a conventional procedure.

In a particular embodiment, the EGFR antagonist is a compound according to formula II′ as disclosed in U.S. Pat. No. 5,770,599, incorporated herein by reference:

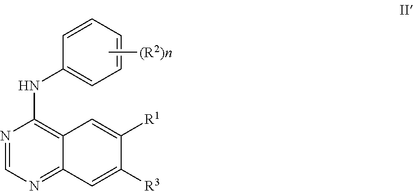

wherein:

n is 1, 2 or 3;

each R² is independently halogeno or trifluoromethyl

R³ is (1-4C)alkoxy; and

R¹ is di-[(1-4C)alkyl]amino-(2-4C)alkoxy, pyrrolidin-1-yl-(2-4C)alkoxy, piperidino-(2-4C)alkoxy, morpholino-(2-4C)alkoxy, piperazin-1-yl-(2-4C)alkoxy, 4-(1-4C)alkylpiperazin-1-yl-(2-4C)alkoxy, imidazol-1-yl-(2-4C)alkoxy, di-[(1-4C)alkoxy-(2-4C)alkyl]amino-(2-4C)alkoxy, thiamorpholino-(2-4C)alkoxy, 1-oxothiamorpholino-(2-4C)alkoxy or 1,1-dioxothiamorpholino-(2-4C)alkoxy, and wherein any of the above mentioned R¹ substituents comprising a CH₂ (methylene) group which is not attached to a N or O atom optionally bears on said CH₂ group a hydroxy substituent;

or a pharmaceutically-acceptable salt thereof.

In a particular embodiment, the EGFR antagonist is a compound according to formula II′ selected from the group consisting of: 4-(3′-chloro-4′-fluoroanilino)-7-methoxy-6-(2-pyrrolidin-1-ylethoxy)-quinazoline; 4-(3′-chloro-4′-fluoroanilino)-7-methoxy-6-(2-morpholinoethoxy)-quinazoline; 4-(3′-chloro-4′-fluoroanilino)-6-(3-diethylaminopropoxy)-7-methoxyquinazoline; 4-(3′-chloro-4′-fluoroanilino)-7-methoxy-6-(3-pyrrolidin-1-ylpropoxy)-quinazoline; 4-(3′-chloro-4′-fluoroanilino)-6-(3-dimethylaminopropoxy)-7-methoxyquinazoline; 4-(3′,4′-difluoroanilino)-7-methoxy-6-(3-morpholinopropoxy)-quinazoline; 4-(3′-chloro-4′-fluoroanilino)-7-methoxy-6-(3-piperidinopropoxy)-quinazoline; 4-(3′-chloro-4′-fluoroanilino)-7-methoxy-6-(3-morpholinopropoxy)-quinazoline; 4-(3′-chloro-4′-fluoroanilino)-6-(2-dimethylaminoethoxy)-7-methoxyquinazoline; 4-(2′,4′-difluoroanilino)-6-(3-dimethylaminopropoxy)-7-methoxyquinazoline; 4-(2′,4′-difluoroanilino)-7-methoxy-6-(3-morpholinopropoxy)-quinazoline; 4-(3′-chloro-4′-fluoroanilino)-6-(2-imidazol-1-ylethoxy)-7-methoxyquinazoline; 4-(3′-chloro-4′-fluoroanilino)-6-(3-imidazol-1-ylpropoxy)-7-methoxyquinazoline; 4-(3′-chloro-4′-fluoroanilino)-6-(2-dimethylaminoethoxy)-7-methoxyquinazoline; 4-(2′,4′-difluoroanilino)-6-(3-dimethylaminopropoxy)-7-methoxyquinazoline; 4-(2′,4′-difluoroanilino)-7-methoxy-6-(3-morpholinopropoxy)-quinazoline; 4-(3′-chloro-4′-fluoroanilino)-6-(2-imidazol-1-ylethoxy)-7-methoxyquinazoline; and 4-(3′-chloro-4′-fluoroanilino)-6-(3-imidazol-1-ylpropoxy)-7-methoxyquinazoline.

In a particular embodiment, the EGFR antagonist is a compound according to formula II′ that is 4-(3′-chloro-4′-fluoroanilino)-7-methoxy-6-(3-morpholinopropoxy)-quinazoline, alternatively referred to as ZD 1839, gefitinib and Iressa®.

A quinazoline derivative of the formula II′, or a pharmaceutically-acceptable salt thereof, may be prepared by any process known to be applicable to the preparation of chemically-related compounds. Suitable processes include, for example, those illustrated in U.S. Pat. No. 5,616,582, U.S. Pat. No. 5,580,870, U.S. Pat. No. 5,475,001 and U.S. Pat. No. 5,569,658. Unless otherwise stated, n, R², R³ and R¹ have any of the meanings defined hereinbefore for a quinazoline derivative of the formula II′. Necessary starting materials may be commercially available or obtained by standard procedures of organic chemistry.

(a) The reaction, conveniently in the presence of a suitable base, of a quinazoline (iii) wherein

Z is a displaceable group, with an aniline (iv)

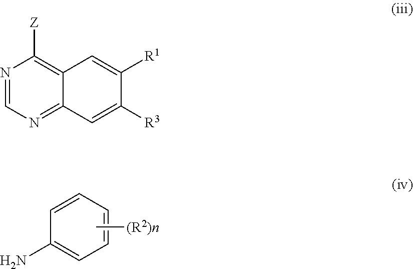

A suitable displaceable group Z is, for example, a halogeno, alkoxy, aryloxy or sulphonyloxy group, for example a chloro, bromo, methoxy, phenoxy, methanesulphonyloxy or toluene-4-sulphonyloxy group.

A suitable base is, for example, an organic amine base such as, for example, pyridine, 2,6-lutidine, collidine, 4-dimethylaminopyridine, triethylamine, morpholine, N-methylmorpholine or diazabicyclo[5.4.0]undec-7-ene, or for example, an alkali or alkaline earth metal carbonate or hydroxide, for example sodium carbonate, potassium carbonate, calcium carbonate, sodium hydroxide or potassium hydroxide. Alternatively a suitable base is, for example, an alkali metal or alkaline earth metal amide, for example sodium amide or sodium bis(trimethylsilyl)amide.

The reaction is preferably carried out in the presence of a suitable inert solvent or diluent, for example an alkanol or ester such as methanol, ethanol, isopropanol or ethyl acetate, a halogenated solvent such as methylene chloride, chloroform or carbon tetrachloride, an ether such as tetrahydrofuran or 1,4-dioxan, an aromatic solvent such as toluene, or a dipolar aprotic solvent such as N,N-dimethylformamide, N,N-dimethylacetamide, N-methylpyrrolidin-2-one or dimethylsulphoxide. The reaction is conveniently carried out at a temperature in the range, for example, 10° to 150° C., preferably in the range 20° to 80° C.

The quinazoline derivative of the formula II′ may be obtained from this process in the form of the free base or alternatively it may be obtained in the form of a salt with the acid of the formula H—Z wherein Z has the meaning defined hereinbefore. When it is desired to obtain the free base from the salt, the salt may be treated with a suitable base as defined hereinbefore using a conventional procedure.

(b) For the production of those compounds of the formula II′ wherein R¹ is an amino-substituted (2-4C)alkoxy group, the alkylation, conveniently in the presence of a suitable base as defined hereinbefore, of a quinazoline derivative of the formula II′ wherein R¹ is a hydroxy group.

A suitable alkylating agent is, for example, any agent known in the art for the alkylation of hydroxy to amino-substituted alkoxy, for example an amino-substituted alkyl halide, for example an amino-substituted (2-4C)alkyl chloride, bromide or iodide, in the presence of a suitable base as defined hereinbefore, in a suitable inert solvent or diluent as defined hereinbefore and at a temperature in the range, for example, 10° to 140° C., conveniently at or near 80° C.

(c) For the production of those compounds of the formula II′ wherein R¹ is an amino-substituted (2-4C)alkoxy group, the reaction, conveniently in the presence of a suitable base as defined hereinbefore, of a compound of the formula II′ wherein R¹ is a hydroxy-(2-4C)alkoxy group, or a reactive derivative thereof, with an appropriate amine.

A suitable reactive derivative of a compound of the formula II′ wherein R¹ is a hydroxy-(2-4C)alkoxy group is, for example, a halogeno- or sulphonyloxy-(2-4C)alkoxy group such as a bromo- or methanesulphonyloxy-(2-4C)alkoxy group.

The reaction is preferably carried out in the presence of a suitable inert solvent or diluent as defined hereinbefore and at a temperature in the range, for example, 10° to 150° C., conveniently at or near 50° C.

(d) For the production of those compounds of the formula II′ wherein R¹ is a hydroxy-amino-(2-4C)alkoxy group, the reaction of a compound of the formula II′ wherein R¹ is a 2,3-epoxypropoxy or 3,4-epoxybutoxy group with an appropriate amine.

The reaction is preferably carried out in the presence of a suitable inert solvent or diluent as defined hereinbefore and at a temperature in the range, for example, 10° to 150° C., conveniently at or near 70° C.

When a pharmaceutically-acceptable salt of a quinazoline derivative of the formula II′ is required, for example a mono- or di-acid-addition salt of a quinazoline derivative of the formula II′, it may be obtained, for example, by reaction of said compound with, for example, a suitable acid using a conventional procedure.

In a particular embodiment, the EGFR antagonist is a compound according to formula III as disclosed in WO9935146, incorporated herein by reference:

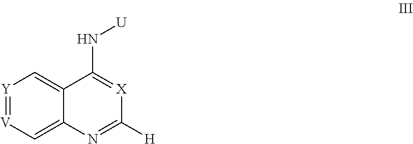

or a salt or solvate thereof; wherein

X is N or CH;

Y is CR¹ and V is N;

or Y is N and V is CR¹;

or Y is CR¹ and V is CR²;

or Y is CR² and V is CR¹;

R¹ represents a group CH₃SO₂CH₂CH₂NHCH₂—Ar—, wherein Ar is selected from phenyl, furan, thiophene, pyrrole and thiazole, each of which may optionally be substituted by one or two halo, C₁₋₄alkyl or C₁₋₄alkoxy groups;

R² is selected from the group comprising hydrogen, halo, hydroxy, C₁₋₄alkyl, C₁₋₄alkoxy, C₁₋₄alkylamino and di[C₁₋₄alkyl]amino;

U represents a phenyl, pyridyl, 3H-imidazolyl, indolyl, isoindolyl, indolinyl, isoindolinyl, 1H-indazolyl, 2,3-dihydro-1H-indazolyl, 1H-benzimidazolyl, 2,3-dihydro-1H-benzimidazolyl or 1H-benzotriazolyl group, substituted by an R³ group and optionally substituted by at least one independently selected R⁴ group;

R³ is selected from a group comprising benzyl, halo-, dihalo- and trihalobenzyl, benzoyl, pyridylmethyl, pyridylmethoxy, phenoxy, benzyloxy, halo-, dihalo- and trihaoobenzyloxy and benzenesulphonyl; or R³ represents trihalomethylbenzyl or trihalomethylbenzyloxy;

or R³ represents a group of formula

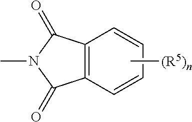

wherein each R⁵ is independently selected from halogen, C₁₋₄alkyl and C₁₋₄alkoxy; and n is O to 3; and

each R⁴ is independently hydroxy, halogen, C₁₋₄alkyl, C₂₋₄alkenyl, C2-4alkynyl, C₁₋₄alkoxy, amino, C₁₋₄alkylamino, di[C₁₋₄alkyl]amino, C1-4alkylthio, C1-4alkylsulphinyl, C₁₋₄alkylsulphonyl, C₁₋₄alkylcarbonyl, carboxy, carbamoyl, C₁₋₄alkoxycarbonyl, C₁₋₄ alkanoylamino, N—(C₁₋₄alkyl)carbamoyl, N,N-di(C₁₋₄alkyl)carbamoyl, cyano, nitro and trifluoromethyl.

In a particular embodiment, EGFR antagonists of formula III exclude: (1-Benzyl-1H-indazol-5-yl)-(6-(5-((2-methanesulphonyl-ethylamino)-methyl)-furan-2-yl)-pyrido[3,4-d]pyrimidin-4-yl-amine; (4-Benzyloxy-phenyl)-(6-(5-((2-methanesulphonyl-ethylamino)-methyl)-furan-2-yl)-pyrido[3,4-d]pyrimidin-4-yl-amine; (1-Benzyl-1H-indazol-5-yl)-(6-(5-((2-methanesulphonyl-ethylamino)-methyl)-furan-2-yl)-quinazolin-4-yl-amine; (1-Benzyl H-indazol-5-yl)-(7-(5-((2-methanesulphonyl-ethylamino)-methyl)-furan-2-yl)-quinazolin-4-yl-amine; and (1-Benzyl-1H-indazol-5-yl)-(6-(5-((2-methanesulphonyl-ethylamino)-methyl)-1-methyl-pyrrol-2-yl)-quinazolin-4-yl-amine

In a particular embodiment, the EGFR antagonist of formula III are selected from the group consisting of: 4-(4-Fluorobenzyloxy)-phenyl)-(6-(5-((2-methanesulphonyl-ethylamino)methyl)-furan-2-yl)-pyrido[3,4-d]pyrimidin-4-yl)-amine; (4-(3-Fluorobenzyloxy)-phenyl)-(6-(5-((2-methanesulphonyl-ethylamino)methyl)furan-2-yl)-pyrido[3,4-d]pyrimidin-4-yl)-amine; (4-Benzenesulphonyl-phenyl)-(6-(5-((2-methanesulphonyl-ethylamino)-methyl)-furan-2-yl)-pyrido[3,4-d]pyrimidin-4-yl)-amine; (4-Benzyloxy-phenyl)-(6-(3-((2-methanesulphonyl-ethylamino)-methyl)-phenyl)-pyrido[3,4-d]pyrimidin-4-yl)-amine; (4-Benzyloxy-phenyl)-(6-(5-((2-methanesulphonyl-ethylamino)-methyl)-furan-2-yl)quinazolin-4-yl)-amine; (4-(3-Fluorobenzyloxy-phenyl)-(6-(4-((2-methanesulphonyl-ethylamino)-methyl)-furan-2-yl)-pyrido[3,4-d]pyrimidin-4-yl)-amine; (4-Benzyloxy-phenyl)-(6-(2-((2-methanesulphonylethylamino)-methyl)-thiazol-4-yl)quinazolin-4-yl)-amine; N-{4-[(3-Fluorobenzyl)oxy]phenyl}-6-[5-({[2-(methanesulphonyl)ethyl]amino}methyl)-2-furyl]-4-quinazolinamine; N-{4-[(3-Fluorobenzyl)oxy]-3-methoxyphenyl}-6-[5-({[2-(methanesulphonyl)ethyl]amino}methyl)-2-furyl]-4-quinazolinamine; N-[4-(Benzyloxy)phenyl]-7-methoxy-6-[5-({[2-(methanesulphonyl)ethyl]amino}methyl)-2-furyl]-4-quinazolinamine; N-[4-(Benzyloxy)phenyl]-6-[4-({[2-(methanesulphonyl)ethyl]amino}methyl)-2-furyl]-4-quinazolinamine; N-{4-[(3-Fluorobenzyl)oxy]-3-methoxyphenyl}-6-[2-({[2-(methanesulphonyl)ethyl]amino}methyl)-1,3-thiazol-4-yl]-4-quinazolinamine; N-{4-[(3-Bromobenzyl)oxy]phenyl}-6-[2-({[2-(methanesulphonyl)ethyl]amino}methyl)-1,3-thiazol-4-yl]-4-quinazolinamine; N-{4-[(3-Fluorobenzyl)oxy]phenyl)-6-[2-({[2-(methanesulphonyl)ethyl]amino}methyl) 1,3-thiazol-4-yl]-4-quinazolinamine; N-[4-(Benzyloxy)-3-fluoropheny-1]-6-[2-({[2-(methanesulphonyl)ethyl]amino)methyl)-1,3-thiazol-4-yl]-4-quinazolinamine; N-(1-Benzyl-1H-indazol-5-yl)-7-methoxy-6-[5-({[2-(methanesulphonyl)ethyl]amino)methyl)-2-furyl]-4-quinazolinamine; 6-[5-({[2-(Methanesulphonyl)ethyl]amino)methyl)-2-furyl]-N-(4-{[3-(trifluoromethyl)benzyl]oxy)phenyl)-4-quinazolinamine; N-{3-Fluoro-4-[(3-fluorobenzyl)oxy]phenyl}-6-[5-({[2-(methanesulphonyl)ethyl]amino)methyl)-2-furyl]-4-quinazolinamine; N-{4-[(3-Bromobenzyl)oxy]phenyl)-6-[5-({[2-(methanesulphonyl)ethyl]amino)methyl)-2-furyl]-4-quinazolinamine; N-[4-(Benzyloxy)phenyl]-6-[3-({[2-(methanesulphonyl)ethyl]amino}methyl)-2-furyl]-4-quinazolinamine; N-[1-(3-Fluorobenzyl)-1H-indazol-5-yl]-6-[2-({[2-(methanesulphonyl)ethyl]amino}methyl)-1,3-thiazol-4-yl]-4-quinazolinamine; 6-[5-({[2-(Methanesulphonyl)ethyl]amino)methyl)-2-furyl]-N-[4-(benzenesulphonyl)phenyl]-4-quinazolinamine; 6-[2-({[2-(Methanesulphonyl)ethyl]amino)methyl)-1,3-thiazol-4-yl]-N-[4-(benzenesulphonyl)phenyl]-4-quinazolinamine; 6-[2-({[2-(Methanesulphonyl)ethyl]amino}methyl)-1,3-thiazol-4-yl]-N-(4-{[3-(trifluoromethyl)benzyl]oxy)phenyl)-4-quinazolinamine; N-{3-fluoro-4-[(3-fluorobenzyl)oxy]phenyl)-6-[2-({[2-(methanesulphonyl)ethyl]amino}methyl)-1,3-thiazol-4-yl]-4-quinazolinamine; N-(1-Benzyl-1H-indazol-5-yl)-6-[2-({[2-(methanesulphonyl)ethyl]amino)methyl)-1,3-thiazol-4-yl]-4-quinazolinamine; N-(3-Fluoro-4-benzyloxyphenyl)-6-[2-({[2-(methanesulphonyl)ethyl]amino)methyl)-1,3-thiazol-4-yl]-4-quinazolinamine; N-(3-Chloro-4-benzyloxyphenyl)-6-[2-({[2-(methanesulphonyl)ethyl]amino)methyl)-1,3-thiazol-4-yl]-4-quinazolinamine; N-{3-Chloro-4-[(3-fluorobenzyl)oxy]phenyl}-6-[5-({[2-(methanesulphonyl)ethyl]amino)methyl)-2-furyl]-4-quinazolinamine; 6-[5-({[2-(Methanesulphonyl)ethyl]amino)methyl)-2-furyl]-7-methoxy-N-(4-benzenesulphonyl)phenyl-4-quinazolinamine; N-[4-(Benzyloxy)phenyl]-7-fluoro-6-[5-({[2-(methanesulphonyl)ethyl]amino)methyl)-2-furyl]-4-quinazolinamine; N-(1-Benzyl-1H-indazol-5-yl)-7-fluoro-6-[5-({[2-(methanesulphonyl)ethyl]amino}methyl)-2-furyl]-4-quinazolinamine; N-[4-(Benzenesulphonyl)phenyl]-7-fluoro-6-[5-({[2-(methanesulphonyl)ethyl]amino}methyl)-2-furyl]-4-quinazolinamine; N-(3-Trifluoromethyl-4-benzyloxyphenyl)-6-[5-({[2-(methanesulphonyl)ethyl]amino)methyl)-4-furyl]-4-quinazolinamine; and salts and solvates thereof.

In a particular embodiment, the EGFR antagonist is: N-[3-chloro-4-[(3-fluorophenyl)methoxy]phenyl]-6-[5-[[[2-(methylsulfonyl)ethyl]amino]methyl]-2-furanyl]-4-quinazolinamine ditosylate salt (lapatinib).

In a particular embodiment, the EGFR antagonist is a compound according to formula IV as disclosed in WO0132651, incorporated herein by reference:

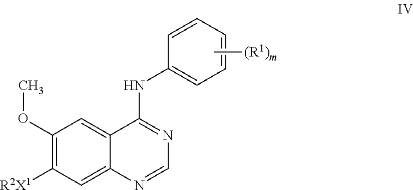

wherein:

m is an integer from 1 to 3;

R¹ represents halogeno or C₁₋₃alkyl;

X¹ represents -0-;

R² is selected from one of the following three groups:

1) C₁₋₅alkylR³ (wherein R³ is piperidin-4-yl which may bear one or two substituents selected from hydroxy, halogeno, C₁₋₄alkyl, C₁₋₄hydroxyalkyl and C₁₋₄alkoxy;

2) C₂₋₅alkenylR³ (wherein R³ is as defined herein);

3) C₂₋₅alkynylR³ (wherein R³ is as defined herein),

and wherein any alkyl, alkenyl or alkynyl group may bear one or more substituents selected from hydroxy, halogeno and amino; or a salt thereof.

In a particular embodiment, the EGFR antagonist is selected from the group consisting of: 4-(4-chloro-2-fluoroanilino)-6-methoxy-7-(1-methylpiperidin-4-ylmethoxy)quinazoline; 4-(2-fluoro-4-methylanilino)-6-methoxy-7-(1-methylpiperidin-4-ylmethoxy)quinazoline; 4-(4-bromo-2-fluoroanilino)-6-methoxy-7-(1-methylpiperidin-4-ylmethoxy)quinazoline; 4-(4-chloro-2,6-difluoroanilino)-6-methoxy-7-(1-methylpiperidin-4-ylmethoxy)quinazoline; 4-(4-bromo-2,6-difluoroanilino)-6-methoxy-7-(1-methylpiperidin-4-ylmethoxy)quinazoline; 4-(4-chloro-2-fluoroanilino)-6-methoxy-7-(piperidin-4-ylmethoxy)quinazoline; 4-(2-fluoro-4-methylanilino)-6-methoxy-7-(piperidin-4-ylmethoxy)quinazoline; 4-(4-bromo-2-fluoroanilino)-6-methoxy-7-(piperidin-4-ylmethoxy)quinazoline; 4-(4-chloro-2,6-difluoroanilino)-6-methoxy-7-(piperidin-4-ylmethoxy)quinazoline; 4-(4-bromo-2,6-difluoroanilino)-6-methoxy-7-(piperidin-4-ylmethoxy)quinazoline; and pharmaceutically acceptable salts and solvates thereof.

In a particular embodiment, the EGFR antagonist is 4-(4-bromo-2-fluoroanilino)-6-methoxy-7-(1-methylpiperidin-4-ylmethoxy)quinazoline (Zactima) and salts thereof

### VEGF Antagonists

In pre-clinical animal models, treatment with the combination of c-met antibody (such as MetMAb), EGFR antagonist (such as erlotinib) and VEGF antagonist (such as an anti-VEGF antibody) resulted in significant improvements in tumor growth inhibition and tumor progression relative to treatment with MetMAb or erlotinib alone or anti-VEGF antibody alone. See co-owned, co-pending U.S. Ser. No. 61/106,513, filed Oct. 17, 2008). Accordingly the invention provides further treatment with a VEGF antagonist.

A VEGF antagonist refers to a molecule capable of binding to VEGF, reducing VEGF expression levels, or neutralizing, blocking, inhibiting, abrogating, reducing, or interfering with VEGF biological activities, including VEGF binding to one or more VEGF receptors and VEGF mediated angiogenesis and endothelial cell survival or proliferation. Included as VEGF antagonists useful in the methods of the invention are polypeptides that specifically bind to VEGF, anti-VEGF antibodies and antigen-binding fragments thereof, receptor molecules and derivatives which bind specifically to VEGF thereby sequestering its binding to one or more receptors, fusions proteins (e.g., VEGF-Trap (Regeneron)), and VEGF₁₂₁-gelonin (Peregrine). VEGF antagonists also include antagonistic variants of VEGF polypeptides, RNA aptamers and peptibodies against VEGF. Examples of each of these are described below.

Anti-VEGF antibodies that are useful in the methods of the invention include any antibody, or antigen binding fragment thereof, that bind with sufficient affinity and specificity to VEGF and can reduce or inhibit the biological activity of VEGF. An anti-VEGF antibody will usually not bind to other VEGF homologues such as VEGF-B or VEGF-C, nor other growth factors such as P1GF, PDGF, or bFGF. Examples of such anti-VEGF antibodies include, but not limited to, those provided herein under “Definitions.”

The two best characterized VEGF receptors are VEGFR1 (also known as Flt-1) and VEGFR2 (also known as KDR and FLK-1 for the murine homolog). The specificity of each receptor for each VEGF family member varies but VEGF-A binds to both Flt-1 and KDR. The full length Flt-1 receptor includes an extracellular domain that has seven Ig domains, a transmembrane domain, and an intracellular domain with tyrosine kinase activity. The extracellular domain is involved in the binding of VEGF and the intracellular domain is involved in signal transduction.

VEGF receptor molecules or fragments thereof that specifically bind to VEGF can be used in the methods of the invention to bind to and sequester the VEGF protein, thereby preventing it from signaling. In certain embodiments, the VEGF receptor molecule, or VEGF binding fragment thereof, is a soluble form, such as sFlt-1. A soluble form of the receptor exerts an inhibitory effect on the biological activity of the VEGF protein by binding to VEGF, thereby preventing it from binding to its natural receptors present on the surface of target cells. Also included are VEGF receptor fusion proteins, examples of which are described below.

A chimeric VEGF receptor protein is a receptor molecule having amino acid sequences derived from at least two different proteins, at least one of which is a VEGF receptor protein (e.g., the flt-1 or KDR receptor), that is capable of binding to and inhibiting the biological activity of VEGF. In certain embodiments, the chimeric VEGF receptor proteins of the present invention consist of amino acid sequences derived from only two different VEGF receptor molecules; however, amino acid sequences comprising one, two, three, four, five, six, or all seven Ig-like domains from the extracellular ligand-binding region of the flt-1 and/or KDR receptor can be linked to amino acid sequences from other unrelated proteins, for example, immunoglobulin sequences. Other amino acid sequences to which Ig-like domains are combined will be readily apparent to those of ordinary skill in the art. Examples of chimeric VEGF receptor proteins include soluble Flt-1/Fc, KDR/Fc, or FLt-1/KDR/Fc (also known as VEGF Trap). (See for example PCT Application Publication No. WO97/44453).

A soluble VEGF receptor protein or chimeric VEGF receptor proteins of the present invention includes VEGF receptor proteins which are not fixed to the surface of cells via a transmembrane domain. As such, soluble forms of the VEGF receptor, including chimeric receptor proteins, while capable of binding to and inactivating VEGF, do not comprise a transmembrane domain and thus generally do not become associated with the cell membrane of cells in which the molecule is expressed.

Aptamers are nucleic acid molecules that form tertiary structures that specifically bind to a target molecule, such as a VEGF polypeptide. The generation and therapeutic use of aptamers are well established in the art. See, e.g., U.S. Pat. No. 5,475,096. A VEGF aptamer is a pegylated modified oligonucleotide, which adopts a three-dimensional conformation that enables it to bind to extracellular VEGF. One example of a therapeutically effective aptamer that targets VEGF for treating age-related macular degeneration is pegaptanib (Macugen™, OSI). Additional information on aptamers can be found in U.S. Patent Application Publication No. 20060148748.

A peptibody is a peptide sequence linked to an amino acid sequence encoding a fragment or portion of an immunoglobulin molecule. Polypeptides may be derived from randomized sequences selected by any method for specific binding, including but not limited to, phage display technology. In one embodiment, the selected polypeptide may be linked to an amino acid sequence encoding the Fc portion of an immunoglobulin. Peptibodies that specifically bind to and antagonize VEGF are also useful in the methods of the invention.

### Therapies

The present invention features the combination use of an anti-c-met antibody and an EGFR antagonist as part of a specific treatment regimen intended to provide a beneficial effect from the combined activity of these therapeutic agents. The beneficial effect of the combination includes, but is not limited to, pharmacokinetic or pharmacodynamic co-action resulting from the combination of therapeutic agents. The present invention is particularly useful in treating cancers of various types at various stages.

The present invention features the use of an anti-c-met antibody as part of a specific treatment regimen intended to provide a beneficial effect from the activity of this therapeutic agent.

In one aspect, the invention provides methods of treating cancer in a subject, comprising administering to the subject an anti-c-met antibody at a dose of about 15 mg/kg every three weeks.

In another aspect, the invention provides methods of treating cancer in a subject, comprising administering to the subject (a) an anti-c-met antibody at a dose of about 15 mg/kg every three weeks; and (b) an EGFR antagonist.

In one aspect, the invention provides methods for extending time to disease progression (TTP), progression free survival or survival in a subject with non-small cell lung cancer, the method comprising administering to the subject (a) an anti-c-met antibody at a dose of about 15 mg/kg every three weeks; and (b) an EGFR antagonist.

In some embodiments, the anti-c-met antibody is administered in an amount sufficient to achieve a serum trough concentration at or above 15 micrograms/ml. In some embodiments, the anti-c-met antibody is administered at a dose of about 15 mg/kg or higher every three weeks. In some embodiments, the anti-c-met antibody is administered at a dose of about 15-20 mg/kg every three weeks.

In some embodiments, the anti-c-met antibody is administered in a total dose of about 15 mg/kg or higher over a three week period.

In one embodiment, the EGFR antagonist is erlotinib. Erlotinib may be administered at a dose of 150 mg, each day of a three week cycle. In some embodiments, erlotinib is administered at a dose of 100 mg. in some embodiments, erlitinib is administered at a dose of 50 mg. Dose reductions of erlotinib are contemplated as indicated on the erlotinib label.

The invention contemplates that multiple series of doses will be administered. When a series of doses is administered, these may, for example, be administered approximately every week, approximately every 2 weeks, approximately every 3 weeks, or approximately every 4 weeks. Multiple series of doses may be administered, for example, two cycles, three cycles, four cycles, or more (5, 6, 7, 8, 9 or more cycles).

In one embodiment, the invention provides methods for extending time to disease progression (TTP), progression free survival or survival in a subject with non-small cell lung cancer, the method comprising administering to the subject (a) an anti-c-met antibody at a dose of about 15 mg/kg every three weeks; and (b) erlotinib (N-(3-ethynylphenyl)-6,7-bis(2-methoxyethoxy)-4-quinazolinamine) at a dose of 150 mg, each day of a three week cycle.

Examples of various cancers that can be treated with an anti-c-met antibody and/or an anti-c-met antibody in combination with an EGFR antagonist are listed in the definition section above. In some embodiments, cancer indications include non-small cell lung cancer, renal cell cancer, pancreatic cancer, gastric carcinoma, bladder cancer, esophageal cancer, mesothelioma, melanoma, breast cancer, thyroid cancer, colorectal cancer, head and neck cancer, osteosarcoma, prostate cancer, or glioblastoma.

Therapy with the anti-c-met antibody, such as MetMAb (in some embodiments, in combination with the EGFR antagonist, such as erlotinib) extends TTP and/or progression free survival and/or survival.

The term cancer embraces a collection of proliferative disorders, including but not limited to pre-cancerous growths, benign tumors, and malignant tumors. Benign tumors remain localized at the site of origin and do not have the capacity to infiltrate, invade, or metastasize to distant sites. Malignant tumors will invade and damage other tissues around them. They can also gain the ability to break off from the original site and spread to other parts of the body (metastasize), usually through the bloodstream or through the lymphatic system where the lymph nodes are located. Primary tumors are classified by the type of tissue from which they arise; metastatic tumors are classified by the tissue type from which the cancer cells are derived. Over time, the cells of a malignant tumor become more abnormal and appear less like normal cells. This change in the appearance of cancer cells is called the tumor grade, and cancer cells are described as being well-differentiated (low grade), moderately-differentiated, poorly-differentiated, or undifferentiated (high grade). Well-differentiated cells are quite normal appearing and resemble the normal cells from which they originated. Undifferentiated cells are cells that have become so abnormal that it is no longer possible to determine the origin of the cells.

Cancer staging systems describe how far the cancer has spread anatomically and attempt to put patients with similar prognosis and treatment in the same staging group. Several tests may be performed to help stage cancer including biopsy and certain imaging tests such as a chest x-ray, mammogram, bone scan, CT scan, and MRI scan. Blood tests and a clinical evaluation are also used to evaluate a patient's overall health and detect whether the cancer has spread to certain organs.

To stage cancer, the American Joint Committee on Cancer first places the cancer, particularly solid tumors, in a letter category using the TNM classification system. Cancers are designated the letter T (tumor size), N (palpable nodes), and/or M (metastases). T1, T2, T3, and T4 describe the increasing size of the primary lesion; N0, N1, N2, N3 indicates progressively advancing node involvement; and M0 and M1 reflect the absence or presence of distant metastases.

In the second staging method, also known as the Overall Stage Grouping or Roman Numeral Staging, cancers are divided into stages 0 to IV, incorporating the size of primary lesions as well as the presence of nodal spread and of distant metastases. In this system, cases are grouped into four stages denoted by Roman numerals I through IV, or are classified as “recurrent.” For some cancers, stage 0 is referred to as “in situ” or “T is,” such as ductal carcinoma in situ or lobular carcinoma in situ for breast cancers. High grade adenomas can also be classified as stage 0. In general, stage I cancers are small localized cancers that are usually curable, while stage 1V usually represents inoperable or metastatic cancer. Stage II and III cancers are usually locally advanced and/or exhibit involvement of local lymph nodes. In general, the higher stage numbers indicate more extensive disease, including greater tumor size and/or spread of the cancer to nearby lymph nodes and/or organs adjacent to the primary tumor. These stages are defined precisely, but the definition is different for each kind of cancer and is known to the skilled artisan.

Many cancer registries, such as the NCI's Surveillance, Epidemiology, and End Results Program (SEER), use summary staging. This system is used for all types of cancer. It groups cancer cases into five main categories:

In situ is early cancer that is present only in the layer of cells in which it began.

Localized is cancer that is limited to the organ in which it began, without evidence of spread.

Regional is cancer that has spread beyond the original (primary) site to nearby lymph nodes or organs and tissues.

Distant is cancer that has spread from the primary site to distant organs or distant lymph nodes.

Unknown is used to describe cases for which there is not enough information to indicate a stage.

In addition, it is common for cancer to return months or years after the primary tumor has been removed. Cancer that recurs after all visible tumor has been eradicated, is called recurrent disease. Disease that recurs in the area of the primary tumor is locally recurrent, and disease that recurs as metastases is referred to as a distant recurrence.

The tumor can be a solid tumor or a non-solid or soft tissue tumor. Examples of soft tissue tumors include leukemia (e.g., chronic myelogenous leukemia, acute myelogenous leukemia, adult acute lymphoblastic leukemia, acute myelogenous leukemia, mature B-cell acute lymphoblastic leukemia, chronic lymphocytic leukemia, polymphocytic leukemia, or hairy cell leukemia) or lymphoma (e.g., non-Hodgkin's lymphoma, cutaneous T-cell lymphoma, or Hodgkin's disease). A solid tumor includes any cancer of body tissues other than blood, bone marrow, or the lymphatic system. Solid tumors can be further divided into those of epithelial cell origin and those of non-epithelial cell origin. Examples of epithelial cell solid tumors include tumors of the gastrointestinal tract, colon, breast, prostate, lung, kidney, liver, pancreas, ovary, head and neck, oral cavity, stomach, duodenum, small intestine, large intestine, anus, gall bladder, labium, nasopharynx, skin, uterus, male genital organ, urinary organs, bladder, and skin. Solid tumors of non-epithelial origin include sarcomas, brain tumors, and bone tumors.

Other therapeutic regimens may be combined therewith. For example, a second (third, fourth, etc) chemotherapeutic agent(s) may be administered, wherein the second chemotherapeutic agent is either another, different antimetabolite chemotherapeutic agent, or a chemotherapeutic agent that is not an antimetabolite. For example, the second chemotherapeutic agent may be a taxane (such as taxotere or paclitaxel or docetaxel), an antimetabolite drug (such as gemcitabine or 5-fluorouracil), capecitabine, or platinum-based chemotherapeutic agent (such as carboplatin, cisplatin, or oxaliplatin), anthracycline (such as doxorubicin, including, liposomal doxorubicin), topotecan, pemetrexed, vinca alkaloid (such as vinorelbine), and TLK 286. “Cocktails” of different chemotherapeutic agents may be administered.

Other therapeutic agents that may be combined with the anti-c-met antibody and EGFR antagonist include any one or more of: an antibody directed against a tumor associated antigen; anti-hormonal compound, e.g., an anti-estrogen compound such as tamoxifen, or an aromatase inhibitor; a cardioprotectant (to prevent or reduce any myocardial dysfunction associated with the therapy); a cytokine); an anti-angiogenic agent (especially bevacizumab sold by Genentech under the trademark AVASTINT™); a tyrosine kinase inhibitor such as sunutinib (SUTENT) and sorafenib; a COX inhibitor (for instance a COX-1 or COX-2 inhibitor); non-steroidal anti-inflammatory drug, celecoxib (CELEBREX®); farnesyl transferase inhibitor (for example, Tipifarnib/ZARNESTRA® R115777 available from Johnson and Johnson or Lonafarnib SCH66336 available from Schering-Plough); a mTOR inhibitor such as RAD001 and temsirolimus; an antibody that binds oncofetal protein CA 125 such as Oregovomab (MoAb B43.13); HER2 vaccine (such as HER2AutoVac vaccine from Pharmexia, or APC8024 protein vaccine from Dendreon, or HER2 peptide vaccine from GSK/Corixa); another HER targeting therapy (e.g. trastuzumab, cetuximab, ABX-EGF, EMD7200, gefitinib, erlotinib, panitumumab, CP724714, CI1033, GW572016, IMC-11F8, TAK165, etc); Raf and/or ras inhibitor (see, for example, WO 2003/86467); doxorubicin HCl liposome injection (DOXIL®); topoisomerase I inhibitor such as topotecan; taxane; HER2 and EGFR dual tyrosine kinase inhibitor such as lapatinib/GW572016; TLK286 (TELCYTA®); EMD-7200; a medicament that treats nausea such as a serotonin antagonist, steroid, or benzodiazepine; a medicament that prevents or treats skin rash or standard acne therapies, including topical or oral antibiotic; a medicament that treats or prevents diarrhea; a body temperature-reducing medicament such as acetaminophen, diphenhydramine, or meperidine; hematopoietic growth factor, etc. Suitable dosages for any of the above coadministered agents are those presently used and may be lowered due to the combined action (synergy) of the agent and anti-c-met antibody and EGFR antagonist, or may be raised, e.g., as determined by a treating physician.

In certain embodiments, when used in combination, bevacizumab is administered in the range from about 0.05 mg/kg to about 15 mg/kg. In one embodiment, one or more doses of about 0.5 mg/kg, 1.0 mg/kg, 2.0 mg/kg, 3.0 mg/kg, 4.0 mg/kg, 5.0 mg/kg, 6.0 mg/kg, 7.0 mg/kg, 7.5 mg/kg, 8.0 mg/kg, 9.0 mg/kg, 10 mg/kg or 15 mg/kg (or any combination thereof) may be administered to the subject. Such doses may be administered intermittently, e.g. every day, every three days, every week or every two to three weeks. In another embodiment, when used in combination, bevacizumab is administered intravenously to the subject at 10 mg/kg every other week or 15 mg/kg every three weeks.

In addition to the above therapeutic regimes, the patient may be subjected to surgical removal of cancer cells and/or radiation therapy.

Where the inhibitor is an antibody, preferably the administered antibody is a naked antibody. However, the inhibitor administered may be conjugated with a cytotoxic agent. Preferably, the conjugated inhibitor and/or antigen to which it is bound is/are internalized by the cell, resulting in increased therapeutic efficacy of the conjugate in killing the cancer cell to which it binds. In a preferred embodiment, the cytotoxic agent targets or interferes with nucleic acid in the cancer cell. Examples of such cytotoxic agents include maytansinoids, calicheamicins, ribonucleases and DNA endonucleases.

In some embodiments, the patient herein is subjected to a diagnostic test e.g., prior to and/or during and/or after therapy. Generally, if a diagnostic test is performed, a sample may be obtained from a patient in need of therapy. Where the subject has cancer, the sample may be a tumor sample, or other biological sample, such as a biological fluid, including, without limitation, blood, urine, saliva, ascites fluid, or derivatives such as blood serum and blood plasma, and the like.

In some embodiments, the subject's cancer expresses c-met and/or EGFR. Methods for determining c-met or EGFR expression are known in the art and certain methods are described herein.

In some embodiments, serum from a subject expresses high levels of IL8. In some embodiments, serum from a subject expresses greater than about 150 pg/ml of IL8, or in some embodiments, greater than about 50 pg/ml IL8. In some embodiments, serum from a subject expresses greater than about 10 pg/ml, 20 pg/ml, 30 pg/ml or more of IL8. Methods for determining IL8 serum concentration are known in the art and one method is described in the present Examples.

In some embodiments, serum from a subject expresses high levels of HGF. In some embodiments, serum from a subject expresses greater than about 5,000, 10,000, or 50,000 pg/ml of HGF.

In some embodiments, decreased mRNA or protein expression in a sample, e.g., from a tumor or serum in a patient treated with a c-met antagonist, and in some embodiments, further treated with an EGFR antagonist, is prognostic, e.g. for response to treatment or for c-met antagonist activity, and in some embodiments, for EGFR antagonist activity. In some embodiments, decreased expression of several angiogenic factor, such as interleukin 8 (IL8), vascular endothelial cell growth factor A (VEGFA), EPH receptor A2 (EphA2), Angiopoietin-like4 (Angptl4), and Ephrin B2 (EFNB2), is prognostic, e.g. for response to treatment or for c-met antagonist activity (and in some embodiment, EGFR antagonist activity). Decrease in expression may be determined relative to an untreated sample or with reference to a normal value or relative to the patient's expression level prior to treatment with the c-met antagonist (or treatment with c-met anatagonist and EGFR antagonist).

In some embodiments, decreased HGF or IL8 expression in a sample, e.g., from a tumor or serum in a patient is prognostic, e.g. for response to treatment or for c-met antagonist (and in some embodiment, EGFR antagonist) activity. In one embodiment, a greater than 50% decrease or a greater than 70% decrease (e.g., relative to IL8 expression level in the patient prior to treatment) in IL8 expression in serum indicates response to treatment. Decrease in expression may be determined relative to an untreated sample or with reference to a normal value or relative to the patient's expression level prior to treatment with the c-met antagonist (or treatment with c-met anatagonist and EGFR antagonist).

In some embodiments, increased mRNA or protein expression in a sample, e.g., from a tumor or serum in a patient treated with a c-met antagonist, and in some embodiments, further treated with an EGFR antagonist, is prognostic, e.g. for response to treatment or for c-met antagonist (and in some embodiment, EGFR antagonist) activity. Decrease in expression may be determined relative to an untreated sample or with reference to a normal value or relative to the patient's expression level prior to treatment with the c-met antagonist (or treatment with c-met anatagonist and EGFR antagonist)

In some embodiments, FDG-PET imaging is prognostic, e.g. for response to treatment or for c-met antagonist activity (and in some embodiment, for EGFR antagonist activity).

The biological sample herein may be a fixed sample, e.g. a formalin fixed, paraffin-embedded (FFPE) sample, or a frozen sample.

Various methods for determining expression of mRNA or protein include, but are not limited to, gene expression profiling, polymerase chain reaction (PCR) including quantitative real time PCR (qRT-PCR), microarray analysis, serial analysis of gene expression (SAGE), MassARRAY, Gene Expression Analysis by Massively Parallel Signature Sequencing (MPSS), proteomics, immunohistochemistry (IHC), etc. Preferably mRNA is quantified. Such mRNA analysis is preferably performed using the technique of polymerase chain reaction (PCR), or by microarray analysis. Where PCR is employed, a preferred form of PCR is quantitative real time PCR (qRT-PCR). In one embodiment, expression of one or more of the above noted genes is deemed positive expression if it is at the median or above, e.g. compared to other samples of the same tumor-type. The median expression level can be determined essentially contemporaneously with measuring gene expression, or may have been determined previously.

The steps of a representative protocol for profiling gene expression using fixed, paraffin-embedded tissues as the RNA source, including mRNA isolation, purification, primer extension and amplification are given in various published journal articles (for example: Godfrey et al. J. Molec. Diagnostics 2: 84-91 (2000); Specht et al., Am. J. Pathol. 158: 419-29 (2001)). Briefly, a representative process starts with cutting about 10 microgram thick sections of paraffin-embedded tumor tissue samples. The RNA is then extracted, and protein and DNA are removed. After analysis of the RNA concentration, RNA repair and/or amplification steps may be included, if necessary, and RNA is reverse transcribed using gene specific promoters followed by PCR. Finally, the data are analyzed to identify the best treatment option(s) available to the patient on the basis of the characteristic gene expression pattern identified in the tumor sample examined.

Detection of gene or protein expression may be determined directly or indirectly.

One may determine expression or amplification of c-met and/or EGFR in the cancer (directly or indirectly). Various diagnostic/prognostic assays are available for this. In one embodiment, c-met and/or EGFR overexpression may be analyzed by IHC. Parafin embedded tissue sections from a tumor biopsy may be subjected to the IHC assay and accorded a c-met and/or EGFR protein staining intensity criteria as follows:

Score 0 no staining is observed or membrane staining is observed in less than 10% of tumor cells.

Score 1+ a faint/barely perceptible membrane staining is detected in more than 10% of the tumor cells. The cells are only stained in part of their membrane.

Score 2+ a weak to moderate complete membrane staining is observed in more than 10% of the tumor cells.

Score 3+ a moderate to strong complete membrane staining is observed in more than 10% of the tumor cells.

In some embodiments, those tumors with 0 or 1+ scores for c-met and/or EGFR overexpression assessment may be characterized as not overexpressing c-met and/or EGFR, whereas those tumors with 2+ or 3+ scores may be characterized as overexpressing c-met and/or EGFR.

In some embodiments, tumors overexpressing c-met and/or EGFR may be rated by immunohistochemical scores corresponding to the number of copies of c-met and/or EGFR molecules expressed per cell, and can been determined biochemically:

0=0-10,000 copies/cell,

1+=at least about 200,000 copies/cell,

2+=at least about 500,000 copies/cell,

3+=at least about 2,000,000 copies/cell.

Alternatively, or additionally, FISH assays may be carried out on formalin-fixed, paraffin-embedded tumor tissue to determine the extent (if any) of c-met and/or EGFR amplification in the tumor.

C-met or EGFR activation may be determined directly (e.g., by phospho-ELISA testing, or other means of detecting phosphorylated receptor) or indirectly (e.g., by detection of activated downstream signaling pathway components, detection of receptor dimmers (e.g., homodimers, heterodimers), detection of gene expression profiles and the like.

Similarly, c-met or EGFR constitutive activation or presence of ligand-independent EGFR or c-met may be detected directly or indirectly (e.g., by detection of receptor mutations correlated with constitutive activity, by detection of receptor amplification correlated with constitutive activity and the like).

Methods for detection of nucleic acid mutations are well known in the art. Often, though not necessarily, a target nucleic acid in a sample is amplified to provide the desired amount of material for determination of whether a mutation is present. Amplification techniques are well known in the art. For example, the amplified product may or may not encompass all of the nucleic acid sequence encoding the protein of interest, so long as the amplified product comprises the particular amino acid/nucleic acid sequence position where the mutation is suspected to be.

In one example, presence of a mutation can be determined by contacting nucleic acid from a sample with a nucleic acid probe that is capable of specifically hybridizing to nucleic acid encoding a mutated nucleic acid, and detecting said hybridization. In one embodiment, the probe is detectably labeled, for example with a radioisotope (³H, ³²P, ³³P etc), a fluorescent agent (rhodamine, fluorescene etc.) or a chromogenic agent. In some embodiments, the probe is an antisense oligomer, for example PNA, morpholino-phosphoramidates, LNA or 2′-alkoxyalkoxy. The probe may be from about 8 nucleotides to about 100 nucleotides, or about 10 to about 75, or about 15 to about 50, or about 20 to about 30. In another aspect, nucleic acid probes of the invention are provided in a kit for identifying c-met mutations in a sample, said kit comprising an oligonucleotide that specifically hybridizes to or adjacent to a site of mutation in the nucleic acid encoding c-met. The kit may further comprise instructions for treating patients having tumors that contain c-met mutations with a c-met antagonist based on the result of a hybridization test using the kit.

Mutations can also be detected by comparing the electrophoretic mobility of an amplified nucleic acid to the electrophoretic mobility of corresponding nucleic acid encoding wild-type c-met. A difference in the mobility indicates the presence of a mutation in the amplified nucleic acid sequence. Electrophoretic mobility may be determined by any appropriate molecular separation technique, for example on a polyacrylamide gel.

Nucleic acids may also be analyzed for detection of mutations using Enzymatic Mutation Detection (EMD) (Del Tito et al, Clinical Chemistry 44:731-739, 1998). EMD uses the bacteriophage resolvase T₄ endonuclease VII, which scans along double-stranded DNA until it detects and cleaves structural distortions caused by base pair mismatches resulting from nucleic acid alterations such as point mutations, insertions and deletions. Detection of two short fragments formed by resolvase cleavage, for example by gel eletrophoresis, indicates the presence of a mutation. Benefits of the EMD method are a single protocol to identify point mutations, deletions, and insertions assayed directly from amplification reactions, eliminating the need for sample purification, shortening the hybridization time, and increasing the signal-to-noise ratio. Mixed samples containing up to a 20-fold excess of normal nucleic acids and fragments up to 4 kb in size can been assayed. However, EMD scanning does not identify particular base changes that occur in mutation positive samples, therefore often requiring additional sequencing procedures to identify the specific mutation if necessary. CEL I enzyme can be used similarly to resolvase T₄ endonuclease VII, as demonstrated in U.S. Pat. No. 5,869,245.

Another simple kit for detecting mutations is a reverse hybridization test strip similar to Haemochromatosis StripAssay™ (Viennalabs http://www.bamburghmarrsh.com/pdf/4220.pdf) for detection of multiple mutations in HFE, TFR2 and FPN1 genes causing Haemochromatosis. Such an assay is based on sequence specific hybridization following amplification by PCR. For single mutation assays, a microplate-based detection system may be applied, whereas for multi-mutation assays, test strips may be used as “macro-arrays”. Kits may include ready-to use reagents for sample prep, amplification and mutation detection. Multiplex amplification protocols provide convenience and allow testing of samples with very limited volumes. Using the straightforward StripAssay format, testing for twenty and more mutations may be completed in less than five hours without costly equipment. DNA is isolated from a sample and the target nucleic acid is amplified in vitro (e.g., by PCR) and biotin-labelled, generally in a single (“multiplex”) amplification reaction. The amplification products are then selectively hybridized to oligonucleotide probes (wild-type and mutant specific) immobilized on a solid support such as a test strip in which the probes are immobilized as parallel lines or bands. Bound biotinylated amplicons are detected using streptavidin-alkaline phosphatase and color substrates. Such an assay can detect all or any subset of the mutations of the invention. With respect to a particular mutant probe band, one of three signaling patterns are possible: (i) a band only for wild-type probe which indicates normal nucleic acid sequence, (ii) bands for both wild-type and a mutant probe which indicates heterozygous genotype, and (iii) band only for the mutant probe which indicates homozygous mutant genotype. Accordingly, in one aspect, the invention provides a method of detecting mutations of the invention comprising isolating and/or amplifying a target c-met nucleic acid sequence from a sample, such that the amplification product comprises a ligand, contacting the amplification product with a probe which comprises a detectable binding partner to the ligand and the probe is capable of specifically hydribizing to a mutation of the invention, and then detecting the hybridization of said probe to said amplification product. In one embodiment, the ligand is biotin and the binding partner comprises avidin or streptavidin. In one embodiment, the binding partner comprises steptavidin-alkaline which is detectable with color substrates. In one embodiment, the probes are immobilized for example on a test strip wherein probes complementary to different mutations are separated from one another. Alternatively, the amplified nucleic acid is labelled with a radioisotope in which case the probe need not comprise a detectable label.

Alterations of a wild-type gene encompass all forms of mutations such as insertions, inversions, deletions, and/or point mutations. In one embodiment, the mutations are somatic. Somatic mutations are those which occur only in certain tissues, e.g., in the tumor tissue, and are not inherited in the germ line. Germ line mutations can be found in any of a body's tissues.

A sample comprising a target nucleic acid can be obtained by methods well known in the art, and that are appropriate for the particular type and location of the tumor. Tissue biopsy is often used to obtain a representative piece of tumor tissue. Alternatively, tumor cells can be obtained indirectly in the form of tissues/fluids that are known or thought to contain the tumor cells of interest. For instance, samples of lung cancer lesions may be obtained by resection, bronchoscopy, fine needle aspiration, bronchial brushings, or from sputum, pleural fluid or blood. Mutant genes or gene products can be detected from tumor or from other body samples such as urine, sputum or serum. The same techniques discussed above for detection of mutant target genes or gene products in tumor samples can be applied to other body samples. Cancer cells are sloughed off from tumors and appear in such body samples. By screening such body samples, a simple early diagnosis can be achieved for diseases such as cancer. In addition, the progress of therapy can be monitored more easily by testing such body samples for mutant target genes or gene products.

Means for enriching a tissue preparation for tumor cells are known in the art. For example, the tissue may be isolated from paraffin or cryostat sections. Cancer cells may also be separated from normal cells by flow cytometry or laser capture microdissection. These, as well as other techniques for separating tumor from normal cells, are well known in the art. If the tumor tissue is highly contaminated with normal cells, detection of mutations may be more difficult, although techniques for minimizing contamination and/or false positive/negative results are known, some of which are described hereinbelow. For example, a sample may also be assessed for the presence of a biomarker (including a mutation) known to be associated with a tumor cell of interest but not a corresponding normal cell, or vice versa.

Detection of point mutations in target nucleic acids may be accomplished by molecular cloning of the target nucleic acids and sequencing the nucleic acids using techniques well known in the art. Alternatively, amplification techniques such as the polymerase chain reaction (PCR) can be used to amplify target nucleic acid sequences directly from a genomic DNA preparation from the tumor tissue. The nucleic acid sequence of the amplified sequences can then be determined and mutations identified therefrom. Amplification techniques are well known in the art, e.g., polymerase chain reaction as described in Saiki et al., Science 239:487, 1988; U.S. Pat. Nos. 4,683,203 and 4,683,195.

It should be noted that design and selection of appropriate primers are well established techniques in the art.

The ligase chain reaction, which is known in the art, can also be used to amplify target nucleic acid sequences. See, e.g., Wu et al., Genomics, Vol. 4, pp. 560-569 (1989). In addition, a technique known as allele specific PCR can also be used. See, e.g., Ruano and Kidd, Nucleic Acids Research, Vol. 17, p. 8392, 1989. According to this technique, primers are used which hybridize at their 3′ ends to a particular target nucleic acid mutation. If the particular mutation is not present, an amplification product is not observed. Amplification Refractory Mutation System (ARMS) can also be used, as disclosed in European Patent Application Publication No. 0332435, and in Newton et al., Nucleic Acids Research, Vol. 17, p. 7, 1989. Insertions and deletions of genes can also be detected by cloning, sequencing and amplification. In addition, restriction fragment length polymorphism (RFLP) probes for the gene or surrounding marker genes can be used to score alteration of an allele or an insertion in a polymorphic fragment. Single stranded conformation polymorphism (SSCP) analysis can also be used to detect base change variants of an allele. See, e.g. Orita et al., Proc. Natl. Acad. Sci. USA Vol. 86, pp. 2766-2770, 1989, and Genomics, Vol. 5, pp. 874-879, 1989. Other techniques for detecting insertions and deletions as known in the art can also be used.

Alteration of wild-type genes can also be detected on the basis of the alteration of a wild-type expression product of the gene. Such expression products include both mRNA as well as the protein product. Point mutations may be detected by amplifying and sequencing the mRNA or via molecular cloning of cDNA made from the mRNA. The sequence of the cloned cDNA can be determined using DNA sequencing techniques which are well known in the art. The cDNA can also be sequenced via the polymerase chain reaction (PCR).

Mismatches are hybridized nucleic acid duplexes which are not 100% complementary. The lack of total complementarity may be due to deletions, insertions, inversions, substitutions or frameshift mutations. Mismatch detection can be used to detect point mutations in a target nucleic acid. While these techniques can be less sensitive than sequencing, they are simpler to perform on a large number of tissue samples. An example of a mismatch cleavage technique is the RNase protection method, which is described in detail in Winter et al., Proc. Natl. Acad. Sci. USA, Vol. 82, p. 7575, 1985, and Meyers et al., Science, Vol. 230, p. 1242, 1985. For example, a method of the invention may involve the use of a labeled riboprobe which is complementary to the human wild-type target nucleic acid. The riboprobe and target nucleic acid derived from the tissue sample are annealed (hybridized) together and subsequently digested with the enzyme RNase A which is able to detect some mismatches in a duplex RNA structure. If a mismatch is detected by RNase A, it cleaves at the site of the mismatch. Thus, when the annealed RNA preparation is separated on an electrophoretic gel matrix, if a mismatch has been detected and cleaved by RNase A, an RNA product will be seen which is smaller than the full-length duplex RNA for the riboprobe and the mRNA or DNA. The riboprobe need not be the full length of the target nucleic acid mRNA or gene, but can a portion of the target nucleic acid, provided it encompasses the position suspected of being mutated. If the riboprobe comprises only a segment of the target nucleic acid mRNA or gene, it may be desirable to use a number of these probes to screen the whole target nucleic acid sequence for mismatches if desired.

In a similar manner, DNA probes can be used to detect mismatches, for example through enzymatic or chemical cleavage. See, e.g., Cotton et al., Proc. Natl. Acad. Sci. USA, Vol. 85, 4397, 1988; and Shenk et al., Proc. Natl. Acad. Sci. USA, Vol. 72, p. 989, 1975. Alternatively, mismatches can be detected by shifts in the electrophoretic mobility of mismatched duplexes relative to matched duplexes. See, e.g., Cariello, Human Genetics, Vol. 42, p. 726, 1988. With either riboprobes or DNA probes, the target nucleic acid mRNA or DNA which might contain a mutation can be amplified before hybridization. Changes in target nucleic acid DNA can also be detected using Southern hybridization, especially if the changes are gross rearrangements, such as deletions and insertions.

Target nucleic acid DNA sequences which have been amplified may also be screened using allele-specific probes. These probes are nucleic acid oligomers, each of which contains a region of the target nucleic acid gene harboring a known mutation. For example, one oligomer may be about 30 nucleotides in length, corresponding to a portion of the target gene sequence. By use of a battery of such allele-specific probes, target nucleic acid amplification products can be screened to identify the presence of a previously identified mutation in the target gene. Hybridization of allele-specific probes with amplified target nucleic acid sequences can be performed, for example, on a nylon filter. Hybridization to a particular probe under stringent hybridization conditions indicates the presence of the same mutation in the tumor tissue as in the allele-specific probe.

Alteration of wild-type target genes can also be detected by screening for alteration of the corresponding wild-type protein. For example, monoclonal antibodies immunoreactive with a target gene product can be used to screen a tissue, for example an antibody that is known to bind to a particular mutated position of the gene product (protein). For example, an antibody that is used may be one that binds to a deleted exon (e.g., exon 14) or that binds to a conformational epitope comprising a deleted portion of the target protein. Lack of cognate antigen would indicate a mutation. Antibodies specific for products of mutant alleles could also be used to detect mutant gene product. Antibodies may be identified from phage display libraries. Such immunological assays can be done in any convenient format known in the art. These include Western blots, immunohistochemical assays and ELISA assays. Any means for detecting an altered protein can be used to detect alteration of wild-type target genes.

Primer pairs are useful for determination of the nucleotide sequence of a target nucleic acid using nucleic acid amplification techniques such as the polymerase chain reaction. The pairs of single stranded DNA primers can be annealed to sequences within or surrounding the target nucleic acid sequence in order to prime amplification of the target sequence. Allele-specific primers can also be used. Such primers anneal only to particular mutant target sequence, and thus will only amplify a product in the presence of the mutant target sequence as a template. In order to facilitate subsequent cloning of amplified sequences, primers may have restriction enzyme site sequences appended to their ends. Such enzymes and sites are well known in the art. The primers themselves can be synthesized using techniques which are well known in the art. Generally, the primers can be made using oligonucleotide synthesizing machines which are commercially available. Design of particular primers is well within the skill of the art.

Nucleic acid probes are useful for a number of purposes. They can be used in Southern hybridization to genomic DNA and in the RNase protection method for detecting point mutations already discussed above. The probes can be used to detect target nucleic acid amplification products. They may also be used to detect mismatches with the wild type gene or mRNA using other techniques. Mismatches can be detected using either enzymes (e.g., 51 nuclease), chemicals (e.g., hydroxylamine or osmium tetroxide and piperidine), or changes in electrophoretic mobility of mismatched hybrids as compared to totally matched hybrids. These techniques are known in the art. See Novack et al., Proc. Natl. Acad. Sci. USA, Vol. 83, p. 586, 1986. Generally, the probes are complementary to sequences outside of the kinase domain. An entire battery of nucleic acid probes may be used to compose a kit for detecting mutations in target nucleic acids. The kit allows for hybridization to a large region of a target sequence of interest. The probes may overlap with each other or be contiguous.

If a riboprobe is used to detect mismatches with mRNA, it is generally complementary to the mRNA of the target gene. The riboprobe thus is an antisense probe in that it does not code for the corresponding gene product because it is complementary to the sense strand. The riboprobe generally will be labeled with a radioactive, colorimetric, or fluorometric material, which can be accomplished by any means known in the art. If the riboprobe is used to detect mismatches with DNA it can be of either polarity, sense or anti-sense. Similarly, DNA probes also may be used to detect mismatches.

In some instances, the cancer does or does not overexpress c-met receptor and/or EGFR. Receptor overexpression may be determined in a diagnostic or prognostic assay by evaluating increased levels of the receptorprotein present on the surface of a cell (e.g. via an immunohistochemistry assay; IHC). Alternatively, or additionally, one may measure levels of receptor-encoding nucleic acid in the cell, e.g. via fluorescent in situ hybridization (FISH; see WO98/45479 published October, 1998), southern blotting, or polymerase chain reaction (PCR) techniques, such as real time quantitative PCR (RT-PCR). Aside from the above assays, various in vivo assays are available to the skilled practitioner. For example, one may expose cells within the body of the patient to an antibody which is optionally labeled with a detectable label, e.g. a radioactive isotope, and binding of the antibody to cells in the patient can be evaluated, e.g. by external scanning for radioactivity or by analyzing a biopsy taken from a patient previously exposed to the antibody.

### Formulations, Dosages and Administrations

The therapeutic agents used in the invention will be formulated, dosed, and administered in a fashion consistent with good medical practice. Factors for consideration in this context include the particular disorder being treated, the particular subject being treated, the clinical condition of the individual patient, the cause of the disorder, the site of delivery of the agent, the method of administration, the scheduling of administration, the drug-drug interaction of the agents to be combined, and other factors known to medical practitioners.

Therapeutic formulations are prepared using standard methods known in the art by mixing the active ingredient having the desired degree of purity with optional physiologically acceptable carriers, excipients or stabilizers (Remington's Pharmaceutical Sciences (20^{th} edition), ed. A. Gennaro, 2000, Lippincott, Williams & Wilkins, Philadelphia, Pa.). Acceptable carriers, include saline, or buffers such as phosphate, citrate and other organic acids; antioxidants including ascorbic acid; low molecular weight (less than about 10 residues) polypeptides; proteins, such as serum albumin, gelatin or immunoglobulins; hydrophilic polymers such as polyvinylpyrrolidone, amino acids such as glycine, glutamine, asparagines, arginine or lysine; monosaccharides, disaccharides, and other carbohydrates including glucose, mannose, or dextrins; chelating agents such as EDTA; sugar alcohols such as mannitol or sorbitol; salt-forming counterions such as sodium; and/or nonionic surfactants such as TWEEN™, PLURONICS™, or PEG.

Optionally, but preferably, the formulation contains a pharmaceutically acceptable salt, preferably sodium chloride, and preferably at about physiological concentrations. Optionally, the formulations of the invention can contain a pharmaceutically acceptable preservative. In some embodiments the preservative concentration ranges from 0.1 to 2.0%, typically v/v. Suitable preservatives include those known in the pharmaceutical arts. Benzyl alcohol, phenol, m-cresol, methylparaben, and propylparaben are preferred preservatives. Optionally, the formulations of the invention can include a pharmaceutically acceptable surfactant at a concentration of 0.005 to 0.02%.

The formulation herein may also contain more than one active compound as necessary for the particular indication being treated, preferably those with complementary activities that do not adversely affect each other. Such molecules are suitably present in combination in amounts that are effective for the purpose intended.

The active ingredients may also be entrapped in microcapsule prepared, for example, by coacervation techniques or by interfacial polymerization, for example, hydroxymethylcellulose or gelatin-microcapsule and poly-(methylmethacylate) microcapsule, respectively, in colloidal drug delivery systems (for example, liposomes, albumin microspheres, microemulsions, nano-particles and nanocapsules) or in macroemulsions. Such techniques are disclosed in Remington's Pharmaceutical Sciences, supra.

Sustained-release preparations may be prepared. Suitable examples of sustained-release preparations include semipermeable matrices of solid hydrophobic polymers containing the antibody, which matrices are in the form of shaped articles, e.g., films, or microcapsule. Examples of sustained-release matrices include polyesters, hydrogels (for example, poly(2-hydroxyethyl-methacrylate), or poly(vinylalcohol)), polylactides (U.S. Pat. No. 3,773,919), copolymers of L-glutamic acid and γ ethyl-L-glutamate, non-degradable ethylene-vinyl acetate, degradable lactic acid-glycolic acid copolymers such as the LUPRON DEPOT™ (injectable microspheres composed of lactic acid-glycolic acid copolymer and leuprolide acetate), and poly-D-(−)-3-hydroxybutyric acid. While polymers such as ethylene-vinyl acetate and lactic acid-glycolic acid enable release of molecules for over 100 days, certain hydrogels release proteins for shorter time periods. When encapsulated antibodies remain in the body for a long time, they may denature or aggregate as a result of exposure to moisture at 37° C., resulting in a loss of biological activity and possible changes in immunogenicity. Rational strategies can be devised for stabilization depending on the mechanism involved. For example, if the aggregation mechanism is discovered to be intermolecular S—S bond formation through thio-disulfide interchange, stabilization may be achieved by modifying sulfhydryl residues, lyophilizing from acidic solutions, controlling moisture content, using appropriate additives, and developing specific polymer matrix compositions.

The therapeutic agents of the invention are administered to a human patient, in accord with known methods, such as intravenous administration as a bolus or by continuous infusion over a period of time, by intramuscular, intraperitoneal, intracerobrospinal, subcutaneous, intra-articular, intrasynovial, intrathecal, oral, topical, or inhalation routes. In the case of VEGF antagonists, local administration is particularly desired if extensive side effects or toxicity is associated with VEGF antagonism. An ex vivo strategy can also be used for therapeutic applications. Ex vivo strategies involve transfecting or transducing cells obtained from the subject with a polynucleotide encoding a c-met or EGFR antagonist. The transfected or transduced cells are then returned to the subject. The cells can be any of a wide range of types including, without limitation, hemopoietic cells (e.g., bone marrow cells, macrophages, monocytes, dendritic cells, T cells, or B cells), fibroblasts, epithelial cells, endothelial cells, keratinocytes, or muscle cells.

For example, if the c-met or EGFR antagonist is an antibody, the antibody is administered by any suitable means, including parenteral, subcutaneous, intraperitoneal, intrapulmonary, and intranasal, and, if desired for local immunosuppressive treatment, intralesional administration. Parenteral infusions include intramuscular, intravenous, intraarterial, intraperitoneal, or subcutaneous administration. In addition, the antibody is suitably administered by pulse infusion, particularly with declining doses of the antibody. Preferably the dosing is given by injections, most preferably intravenous or subcutaneous injections, depending in part on whether the administration is brief or chronic.

In another example, the c-met or EGFR antagonist compound is administered locally, e.g., by direct injections, when the disorder or location of the tumor permits, and the injections can be repeated periodically. The c-met or EGFR antagonist can also be delivered systemically to the subject or directly to the tumor cells, e.g., to a tumor or a tumor bed following surgical excision of the tumor, in order to prevent or reduce local recurrence or metastasis.

Administration of the therapeutic agents in combination typically is carried out over a defined time period (usually minutes, hours, days or weeks depending upon the combination selected). Combination therapy is intended to embrace administration of these therapeutic agents in a sequential manner, that is, wherein each therapeutic agent is administered at a different time, as well as administration of these therapeutic agents, or at least two of the therapeutic agents, in a substantially simultaneous manner.

The therapeutic agent can be administered by the same route or by different routes. For example, the EGFR or c-met antagonist in the combination may be administered by intravenous injection while the protein kinase inhibitor in the combination may be administered orally. Alternatively, for example, both of the therapeutic agents may be administered orally, or both therapeutic agents may be administered by intravenous injection, depending on the specific therapeutic agents. The sequence in which the therapeutic agents are administered also varies depending on the specific agents.

The present application contemplates administration of the c-met and/or EGFR antagonist by gene therapy. See, for example, WO96/07321 published Mar. 14, 1996 concerning the use of gene therapy to generate intracellular antibodies.

There are two major approaches to getting the nucleic acid (optionally contained in a vector) into the patient's cells; in vivo and ex vivo. For in vivo delivery the nucleic acid is injected directly into the patient, usually at the site where the antibody is required. For ex vivo treatment, the patient's cells are removed, the nucleic acid is introduced into these isolated cells and the modified cells are administered to the patient either directly or, for example, encapsulated within porous membranes which are implanted into the patient (see, e.g. U.S. Pat. Nos. 4,892,538 and 5,283,187). There are a variety of techniques available for introducing nucleic acids into viable cells. The techniques vary depending upon whether the nucleic acid is transferred into cultured cells in vitro, or in vivo in the cells of the intended host. Techniques suitable for the transfer of nucleic acid into mammalian cells in vitro include the use of liposomes, electroporation, microinjection, cell fusion, DEAE-dextran, the calcium phosphate precipitation method, etc. A commonly used vector for ex vivo delivery of the gene is a retrovirus.

The currently preferred in vivo nucleic acid transfer techniques include transfection with viral vectors (such as adenovirus, Herpes simplex I virus, or adeno-associated virus) and lipid-based systems (useful lipids for lipid-mediated transfer of the gene are DOTMA, DOPE and DC-Chol, for example). In some situations it is desirable to provide the nucleic acid source with an agent that targets the target cells, such as an antibody specific for a cell surface membrane protein or the target cell, a ligand for a receptor on the target cell, etc. Where liposomes are employed, proteins which bind to a cell surface membrane protein associated with endocytosis may be used for targeting and/or to facilitate uptake, e.g. capsid proteins or fragments thereof tropic for a particular cell type, antibodies for proteins which undergo internalization in cycling, and proteins that target intracellular localization and enhance intracellular half-life. The technique of receptor-mediated endocytosis is described, for example, by Wu et al., J. Biol. Chem. 262:4429-4432 (1987); and Wagner et al., Proc. Natl. Acad. Sci. USA 87:3410-3414 (1990). For review of the currently known gene marking and gene therapy protocols see Anderson et al., Science 256:808-813 (1992). See also WO 93/25673 and the references cited therein.

The following are examples of the methods and compositions of the invention. It is understood that various other embodiments may be practiced, given the general description provided above.

### EXAMPLES

### Example 1

### Pre-Clinical MetMAb Pharmacokinetics (PK) and Pharmacodynamics (PD)

This example describes the use of pre-clinical pharmacokinetics (PK) and efficacy data to determine clinical dose selection for c-met antagonist antibody MetMAb.

Materials and Methods

PK studies. PK studies were conducted in mice, rats, and cynomolgus monkeys. MetMAb binds to c-met in cynomolgus monkeys. MetMAb does not bind to c-met in mice and rats.

Female nude mice (nu/nu) (n=3 per time point/group) were given a single intravenous (IV) bolus dose of MetMAb at 3, 10, or 30 mg/kg and an intraperitoneal (IP) dose of MetMAb of 30 mg/kg. Sprague-Dawley rats (n=6) were given a single IV bolus dose of MetMAb at 30 mg/kg, and cynomolgus monkeys (n=4 per group) were given a single IV dose of MetMAb at 0.5, 3, 10, or 30 mg/kg. Serum was collected at various time points and assayed for serum MetMAb concentration using the assays described below.

Efficacy studies. Four efficacy studies were conducted to evaluate PK driver(s) of MetMAb efficacy.

In a dose response study, female nude (nu/nu) mice (age 6-8 weeks) were inoculated subcutaneously (SC) with 5×10⁶ KP4 human pancreatic ductal cell carcinoma cells. Mice (n=10 per group) were treated with a single IV dose of MetMAb at 0, 1, 3, 7.5, 15, 30, 60, or 120 mg/kg when tumors reached a mean volume of 150-250 mm³.

In a dose fractionation study, KP4 xenograft mice (n=10 per group) were given total MetMAb doses of 2.5 mg/kg, 7.5 mg/kg, or 30 mg/kg fractionated into once weekly (Q1W), once every 2 weeks (Q2W), or Q3W regimens. For example, a 30 mg/kg total dose was given as 10 mg/kg Q1W, 15 mg/kg Q2W, or 30 mg/kg Q3W.

For the IV infusion study, MetMAb treatment began when the mean KP4 tumor volumes were ˜300 mm³. Animals received a single IV dose of MetMAb at 0, 1250 or 312.5 ug/mouse or an IV infusion of 1250 or 312.5 ug/mouse MetMAb at 17.36 ug/hour at 20 uL/hr or 4.34 ug/hr at 20 uL/hr into the tail vein over a 3-day period or an IV infusion of 1250 or 312.5 ug/mouse MetMAb at 7.44 ug/hr at 20 uL/hr or 1.86 ug/hr at 20 uL/hr into the tail vein over a 7-day period.

One serum sample was collected from all mice in each group in the IV infusion study and assayed for MetMAb serum concentrations using the assays described below. The expected serum concentration at the time of serum sample collection was estimated based on PK parameters determined in the PK study in non-tumor bearing mice (described above). Serum disposition of MetMAb in non-tumor-bearing mice was biphasic and exhibited dose-proportionality. The following PK parameters were calculated from a two-compartmental model fit to the dose-normalized, naive-pooled observed data: V₁=48.8 mL/kg, V₂=90.7 mL/kg, CLₜ=21.6 mL/day/kg, CL_{d}=190 mL/day/kg, where V₁ is the apparent central volume of distribution, V₂ is the apparent peripheral volume of distribution, CLₜ is total apparent clearance, and CL_{d} is the inter-compartmental clearance. These PK parameters were used to estimate serum concentration by WinNonlin Enterprise Version 5.0.1 (Pharsight Corp., Mountain View, Calif.) for doses tested in IV infusion study.

Human HGF Transgenic C3H-SCID mice (hu-HGF-Tg-SCID) (age 4-8 weeks) (see U.S. Ser. No. 61/044,438, filed 11 Apr. 2008) were inoculated SC with 0.5×10⁶ NCI-H596 human non-small cell lung cancer (NSCLC) cells. Mice (n=10 per group) were treated with a single IP injection of MetMAb at 15, 30, 90, 180, 240, or 360 mg/kg when tumors reached a mean volume of ˜120 mm³. A positive control group was given MetMAb at 30 mg/kg twice a week. This work was completed at the Van Andel Research Institute [Grand Rapids, Mich.] in accordance with the guidelines of their Institutional Animal Care and Use Committee.

In all cases, tumors were measured with calipers throughout the study.

Pharmacokinetic Assays for MetMAb in Mouse Serum and Rat Serum. Two ELISA methods were developed to quantify MetMAb concentrations. A direct ELISA assay was developed for quantification of MetMAb in mouse serum and Sprague-Dawley rat serum. Plates were coated with human c-Met-Fc fusion protein to which samples, standards, and dosing solutions were added. Goat anti-human F(Ab′)₂ horseradish peroxidase (HRP) was used for detection. Tetrapethyl benzidine (TMB) peroxidase substrate was added for signal development. The substrate reaction was stopped with phosphoric acid. The plates were read at an absorbance of 450 nm. For the nude mouse IV infusion study, the direct ELISA to measure MetMAb for cymomolgus monkey PK samples (described below) was used to assay the mouse serum, with the following modifications: a buffer standard curve replaced the 2% cynomolgus monkey serum standard curve, and the minimum samples dilution was 1/1000. The lower limit of quantitation in the assay was 0.47 ng/mL and the upper limit of quantification was 30 ng/mL. The minimum dilution for nude mouse serum samples was 1/10, resulting in a minimum quantifiable concentration of 4.7 ng/mL, with an indefinite upper limit. The minimum dilution for rat serum samples was 1/50, resulting in a minimum quantifiable concentration of 23.5 ng/mL, with an indefinite upper limit.

Pharmacokinetic Assay for MetMAb in Cynomolgus Monkey Serum. A direct ELISA was developed to quantitate MetMAb in cynomolgus monkey serum. Plates were coated with a His-tagged c-met extracellular domain fragment, and diluted samples, standards, and controls were added to the coated plates. F(Ab′)₂ fragmented, goat anti-human IgG Fc antibodies conjugated to HRP were added for detection. TMB peroxidase was stopped with phosphonic acid. The plates were read at an absorbance of 450 nm and 620/630 nm.

The lower limit of quantification of the assay was 1.0 ng/ml and the upper limit of quantification was 32.0 ng/ml. The minimum dilution for neat cynomolgus monkey serum samples was 1/50, resulting in a minimum quantifiable concentration of 50 ng/ml, with an indefinite upper limit.

Mouse, Rat, and Cynomolgus Monkey PK data analysis. Group mean MetMAb serum concentration-time profiles were created on a semilogarithmic plot using nominal time of sample collection (Kaleidagraph Version 3.6, Synergy Software, Reading Pa., or Microsoft Excel 2003, Microsoft Corp., Redmond Wash.). PK parameters were estimated using WinNonlin Enterprise Version 5.0.1 (Pharsight Corp., Mountain View, Calif.). The nominal dose administered for each group was used for modeling. Since a single concentration-time profile for mice was determined for each group, one estimate of each PK parameter was obtained and is reported, along with the standard error (SE) of the fit of each PK parameter. For rats and monkeys, PK parameters are reported as mean (+/−SD).

For IV administrations, a two-compartment elimination model with IV bolus input and first-order elimination was used to describe the observed data (WinNonlin Model 7). Concentrations were weighted using iterative reweighting (1/ŷ²) and the Nelder-Mead minimization algorithm. The following equation was used to calculate concentration over time in Model 7:

C(t)=A·EXP(−α·t)+B·EXP(−β·t)

where t=time in days, A and B refer to the zero-time intercept for each exponential term, and α and β refer to the exponential coefficient for A and B.

The following PK parameters were reported using Model 7:

t_{1/2 β}=half-life associated with the elimination phase (beta half-life)

CL=clearance

V₁=volume of distribution for the central compartment

Vₛₛ=volume of distribution under steady-state conditions

For each dose groups, model selection was based on goodness of fit by visual inspection of the observed versus predicted serum concentration-time profile for each animals, examination of the weighted residuals sum of squares, and examination of the standard error (SE) and coefficient of variation (CV) for each parameter.

Repeat-dose safety study. Cynomolgus monkeys received 13 weekly doses of 0, 3, 10, 30, or 100 mg/kg MetMAb for 12 weeks (13 doses) by IV bolus administration to study the safety and toxocokinetics of MetMAb. Recovery animals were observed for an additional 8 weeks after the final weekly dose.

Safety Factor Calculation. Safety factor was calculated for Dose, AUC, Cₘₐₓ as the ratio at the highest non-severely toxic dose (HNSTD) observed in the single or repeat-dose safety study to the proposed phase I starting dose. The equations are:

Safety factor (SF)_{Dose}=Dose_{Cyno}/Dose_{Human}

SF_{AUC}=AUC_{cyno}/AUC_{Human}

SF_{Cmax}=(C_{max-Cyno})/(C_{max-Human})

For body surface area calculation: The body weight to surface area index is 12 kg/m² for cynomolgus monkeys and 37.5 kg/m² for humans.

Estimation of Human PK Profile. Two approaches were used to predict MetMAb PK disposition in humans, based on data observed in other smaller species (i.e., mouse, rat, and cynomolgus monkey).

One approach was allometric scaling, which is based on the assumption that many physical and physiological parameters vary according to a mathematical function of body weight (BW)

Y=a×BWb
where Y is the variable of interest, a is the y-axis intercept, and b is slope.

Mean CL values from mice, rats, and cynomolgus monkeys given a single IV bolus dose of MetMAb were used to estimate CL value for human by allometric scaling. The log plot of CL value versus body weight, repression, and R-squared values were generated by KaleidaGraph (version 3.6).

The second approach was a species invariant time method (Gabrielsson, J and Weiner, D, Pharmacokinetic and Pharmacodynamic Data Analysis: Concepts and Applications, 3d Ed., Swedish Pharmaceutical Press, 2000). This method required transformation of animal time to human time using “kallynochrons”, which are units of pharmacokinetic time during which different species clear the same volume of plasma per kilogram BW². The following transformation equations were used for the extrapolation:

$${Time}_{human} = {Time}_{cyno} ( \frac{{Bodyweight}_{human}}{{Bodyweight}_{cyno}} ) {Exponent}_{volume} - {Exponent}_{clearance}$$

$${Concentration}_{human} = {Concentration}_{cyno} ( \frac{{Dose}_{human}}{{Dose}_{cyno}} ) ( \frac{{Bodyweight}_{cyno}}{{Bodyweight}_{human}} ) {Exponent}_{volume}$$

The estimated human serum concentration-time data obtained from cynomolgus monkeys based on above equation were used to estimate the predicted population PK parameters for humans. A scaling exponent of either 0.75 was used to estimate human CL and a scaling exponent of 1 was used to estimate the volume of the central compartment (V₁). The exponent values of 0.75 for CL and 1 for V₁ were based on literature reports (Mahmoud I. J Pharm Sci 2004; 93: 177-85; Tabrizi et al. Drug Discov Today 2006; 11:81-8).

Results

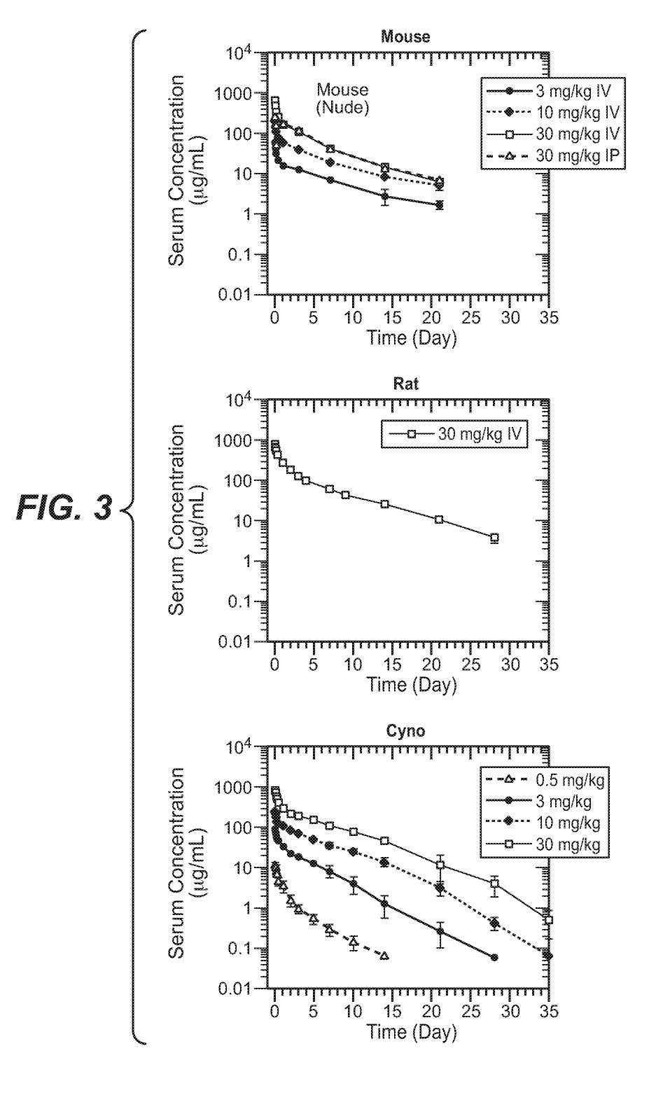

MetMAb clearance (CL) in the linear dose range was approximately 22, 19, and 13 mL/day/kg in mice, rats, and cynomolgus monkeys, respectively. In rodents and cynomolgus monkeys, MetMAb clearance was 2-3 times faster than that typically observed with bivalent glycosylated antibodies which have minimal target-mediated clearance. MetMAb serum concentration-time profiles in mice, rats, and cynomolgus monkeys are shown in FIG. 3, and mean PK parameters are shown in Table 1. Area under the serum concentration-time curve (AUC) and maximum concentration (Cmax) increased in proportion to dose at the dose range of 3-30 mg/kg. Beta half-life ranged from 4-5 days.

TABLE 1
Mean PK Parameters following a Single IV Dose of MetMAb

| | | Terminal | | |
|---|---|---|---|---|
| | CL | Half-Life | V₁ | Vₛₛ |
| Species | (mL/day/kg) | (days) | (mL/kg) | (mL/kg) |
| Athymic nude mouse | 21.6 | 4.70 | 48.8 | 140 |
| (n = 3/time point) | | | | |
| Sprague Dawley rat | 18.8 | 5.18 | 44.6 | 107 |
| (n = 6) | | | | |
| Cynomolgus monkey | 13.4 | 4.05 | 33.8 | 70.3 |
| (n = 4) | | | | |

CL = clearance; V₁ = volume of distribution of the central compartment; Vₛₛ = volume of distribution at steady state

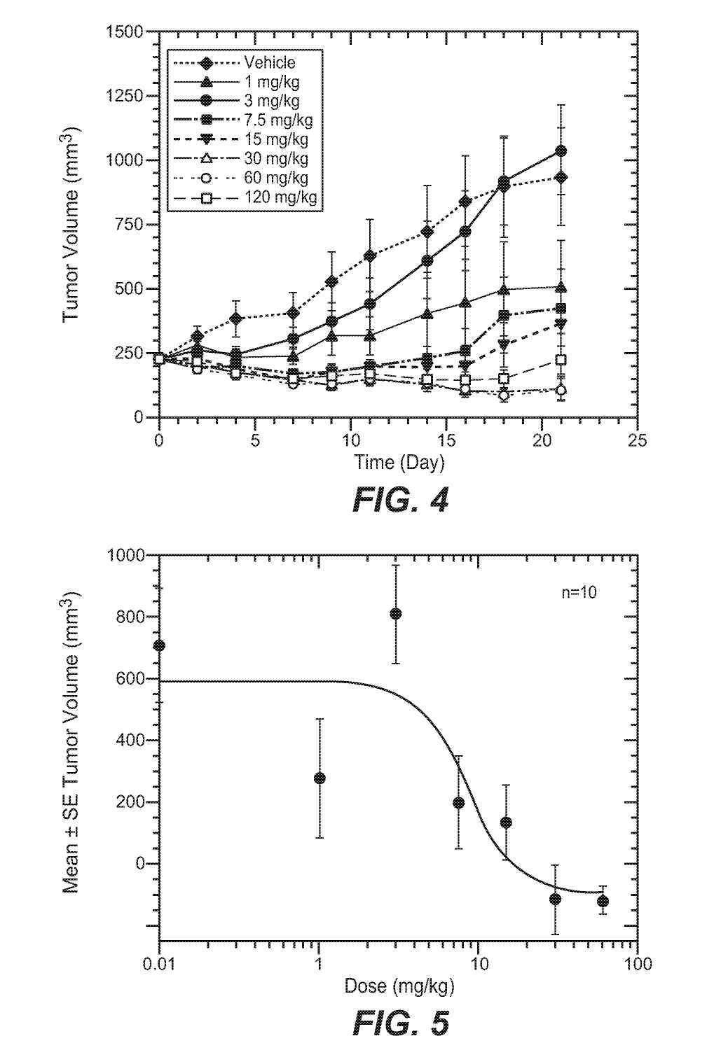

To determine the effective dose 20/50/80 (ED_{20/50/80}), a single dose response study performed to identify minimal, median, and maximal efficacious dose of MetMAb in a KP4 autocrine xenograft model. FIG. 4 shows the results of this experiment. The maximal efficacious MetMAb dose was observed to be greater than or equal to 30 mg/kg.

The mean group tumor volume on Day 21 for each MetMAb dose was used to generate an effect vs. dose profile, shown in FIG. 5. Based on this profile, 2.5, 7.5, and 30 mg/kg were selected as representative of the minimal, median, and maximal efficacious dose for the dose fractionation study.

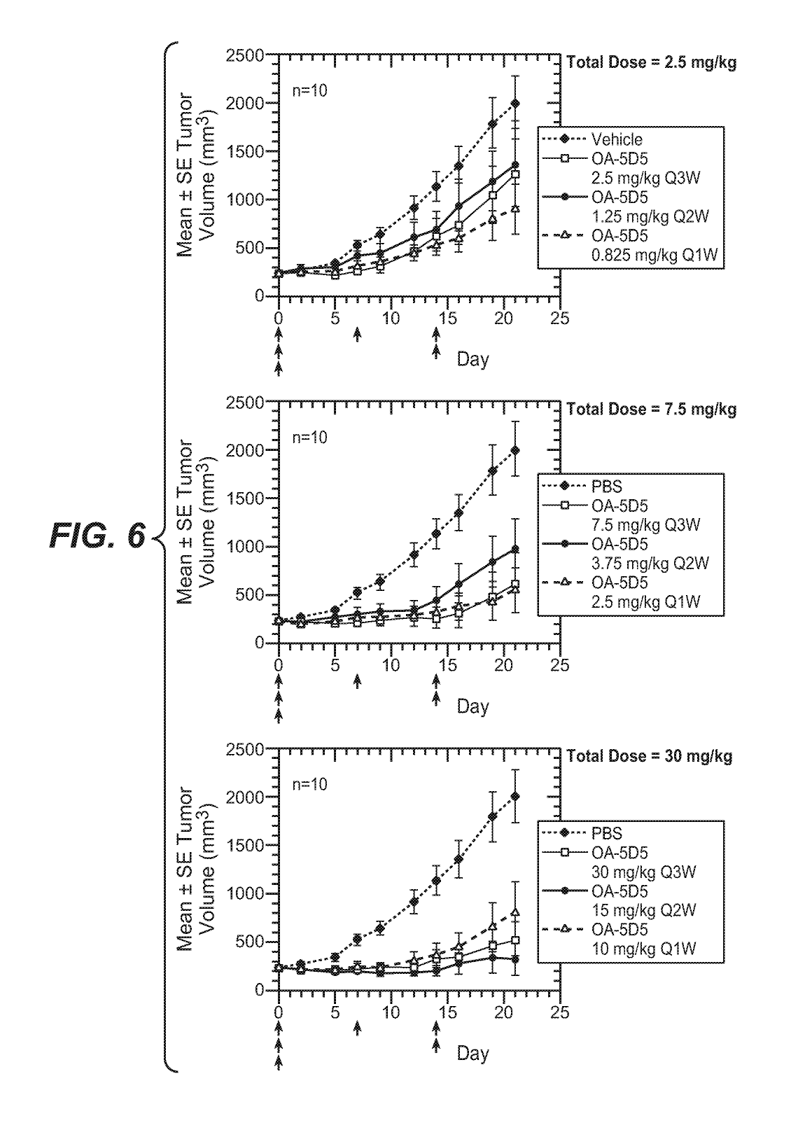

As shown in FIG. 6, the dose fractionation study demonstrated that efficacy at the same dose level with different dose regimens was similar. These results indicated that area under the curve AUC is the PK driver(s) of efficacy for MetMAb. Dosing schedule had a minimal effect on efficacy at the 3 dose levels tested, supporting a clinical dosing regimen of Q1W (once a week) to Q3W (once every three weeks).

To confirm that AUC was the key driver of MetMAb efficacy, an infusion study was conducted as described in the materials and methods. The results of this experiment are shown in FIG. 7. For a given dose of MetMAb administered as a single IV dose or IV infusion to KP4 pancreatic tumor xenograft mice over a 3- or 7-day period, the AUC was similar, but Cₘₐₓ and time above a minimal effective serum concentration were different. IV bolus and IV infusion of MetMAb provided similar efficacy in the KP4 model at 1250 ug/mouse and 312.5 ug/mouse dose levels, respectively. The results of the IV infusion study supported the observation that AUC was the PK driver of MetMAb efficacy. Observed MetMAb serum concentrations in tumor bearing animals were similar to those predicted with PK parameters obtained from non-tumor bearing mice and confirmed the expected MetMAb exposure for IV bolus and IV infusion groups in this study.

MetMAb was also used to treat non-small cell ung cancer (NSCLC) H596 tumors in a hu-HGF-Tg-SCID mouse model. The results of this experiment are shown in FIG. 8. Similar efficacy was observed in all single dose groups in comparison with the repeat-dose of 30 mg/kg twice a week (total of 180 mg/kg in a three week period). Thus, in a paracrine model, MetMAb dose responses were dependent on total dose, not on dosing regimen. MetMAb dose response results observed in this experiment also support dosing at a frequency of once a week to once every three weeks (Q1W-Q3W).

MetMAb clearance was estimated with two methods, allometric scaling and species invariant time. Predicted MetMAb clearance in humans using allometric scaling was 10 mL/day/kg. Predicted MetMAb clearance and half life in humans using species-invariant time was 6.0 mL/day/kg and 9 days, respectively (Table 2).

TABLE 2
Predicted MetMAb Clearance and Half Life in
Humans by Species-Invariant Time Method

| Dose | Clearance | Beta HL |
|---|---|---|
| (mg/kg) | (mL/day/kg) | (day) |
| 3 | 6.9 | 6.6 |
| 10 | 5.4 | 9.4 |
| 30 | 5.6 | 11 |

Beta HL = terminal half life

The good laboratory practice toxicology study identified 100 mg/kg as the highest non-severely toxic dose. The repeat-dose Safety Study in Cynomolgus Monkeys provided a 32- to 115-fold safety margin for a starting IV dose at 1 mg/kg in Phase I in humans.

Human PK parameters used for calculating safety factors were estimated using PK data from cynomolgus monkeys. Single- and multiple-dose safety factors (Table 3) provided greater than thirty-fold safety margin to support a staring dose of 1 mg/kg in Phase I in humans.

TABLE 3
Safety Factors for Planned Phase 1a Clinical Starting Dose Based on
Interspecies Scaling of MetMAb and Body Surface Area Calculation

| | | | | | Multiple Dose Safety | | |
|---|---|---|---|---|---|---|---|
| | Single Dose Safety Factor | | | | Factor | | |
| Planned Phase Ia | | | Body | | | | Body |
| Starting Dose | Single | | Surface | | Total | | Surface |
| (mg/kg) | Dose | AUC | Area | Cₘₐₓ | Dose | AUC | Area |
| 1 | 100 | 113 | 32 | 115 | 325 | 368 | 105 |

AUC = Area under the curve,
Cₘₐₓ = maximum clearance.

Conclusion

MetMAb PK differed from the PK observed with bivalent glycosylated antibodies. After a single IV bolus dose in mice, rats, and monkeys, MetMAb displayed linear PK in the dose range of 3-30 mg/kg. MetMAb clearance was 2-3 fold faster than bivalent glycosylated antibodies which had limited target-mediated clearance.

Efficacy data supported dose flexibility in a clinical setting. The dose fractionation study in a KP4 autocrine xenograft model indicated that AUC is the pharmacokinetic driver of MetMAb efficacy, and the IV infusion study supported that observation. Similar efficacy was observed in non-small cell lung cancer tumors in huHGF-Tg-SCID mouse model.

Allometric scaling and species invariant time methods predicated that the clearance of MetMAb would be in the range of 6.0 to 10 mL/day/kg in a clinical setting.

The repeat-dose safety study in cynomolgus monkeys provide a 32- to 115-fold safety margin for a starting dose at 1 mg/kg in human based on a single dose, which has been approved for clinical safety.

The nonclinical PK and efficacy data summarized in this Example together with the PK/PD modeling approach shown in Example 2 supported the MetMAb clinical dose selection.

### Example 2

### Prediction of a Clinical MetMAb Dose Regimen Using Pre-Clinical and Clinical Data

This example describes the use of modeling and simulation analysis to predict a minimally effective clinical MetMAb dose regimen for objective response using cynomolgus monkey pharmacokinetics (PK) and KP4 xenograft mice anti-tumor efficacy data.

Materials and Methods

A PK study using intravenous administration of MetMAb (3.0, 10.0, and 30.0 mg/kg; n=9/group) was conducted in non-tumor-bearing mice to determine CL, V1, CLd, and V2 PK parameters: V₁=48.8 mL/kg, V₂=90.7 mL/kg, CLₜ=21.6 mL/day/kg, CL_{d}=190 mL/day/kg, where V₁ is the apparent central volume of distribution, V₂ is the apparent peripheral volume of distribution, CLₜ is total apparent clearance, and CL_{d} is the inter-compartmental clearance (Example 1). PK parameter estimates were used as a forcing function to model the pharmacodynamic (PD) endpoint of tumor progression (PD) in KP4 xenograft mice.

For PD data, KP4 xenograft mice (n=10/group) were given a single dose IV MetMAb (1-120 mg/kg) as described in Example 1. Additional KP4 xenograft mice (n=10/group) were given total MetMAb doses (of 2.5 mg/kg, 7.5 mg/kg, and or 30 mg/kg) fractionated by splitting doses over dosing into once weekly (Q1W), dosing every 2 weeks (Q2W), and dosing every 3 weeks (Q3W) regimens, as described in Example 1. Tumor measurements were taken via with calipers, and mice entered the study with tumor volumes of approximately 200 mm³. Study data up to 21 days from a total of 177 KP4 mice were used in the modeling analysis. Tumor volumes (mm³) were converted to mass (mg) assuming 1 mm³=1 mg tumor tissue. A mixed effects PK/pharmacodynamic (PD) model describing anti-tumor efficacy in KP4 xenograft mice was fit to the tumor data using NONMEM software (Double Precision, version V, level 1.0 UCSF, San Francisco Calif.).

To project human MetMAb serum concentrations prior to clinic, cynomolgus monkeys (n=4/group) were given a single dose IV MetMAb (0.5, 3, 10, and 30 mg/kg), and MetMAb concentration-time curves were plotted. Human MetMAb serum concentrations were projected from cynomolgus monkey concentration-time curves using species-invariant time transformations (see equation below) of cynomolgus monkey data (0.5, 3, 10, and 30 mg/kg MetMAb):

$${Time}_{H} = {{Time}_{C} ( \frac{{Bwt}_{H}}{{Bwt}_{C}} )}^{0.25}$$

where H=Human and C=Cyno

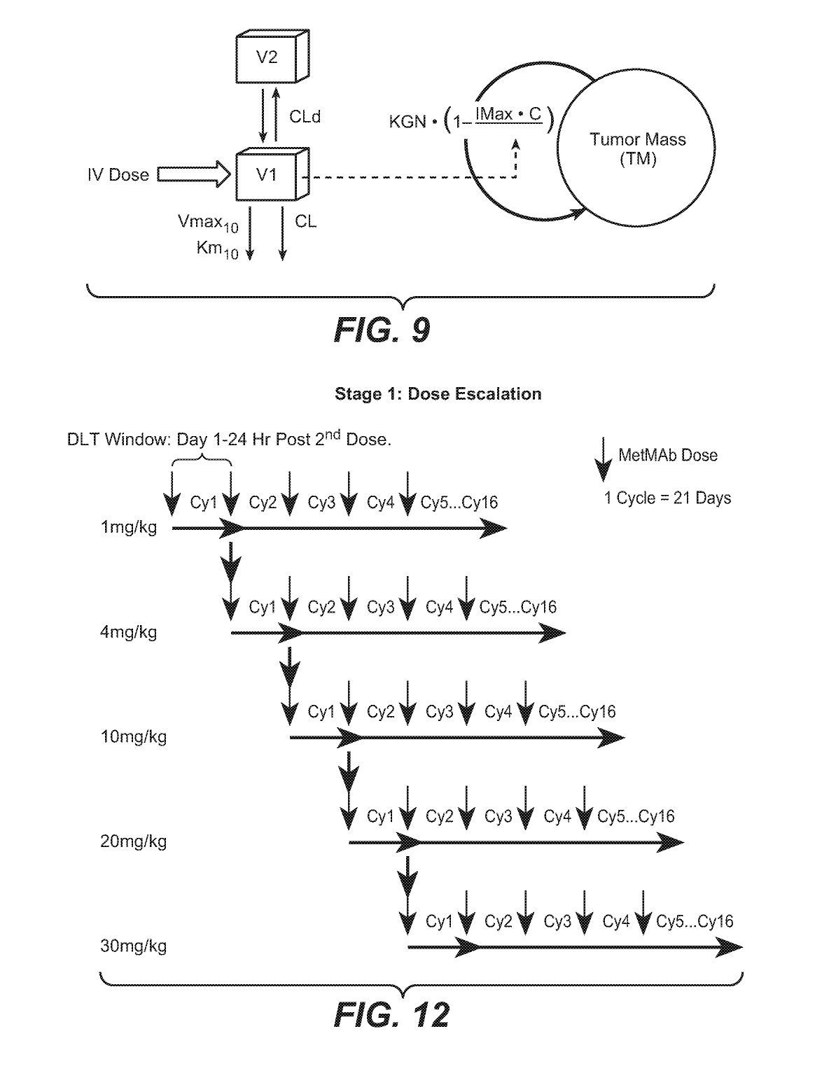

A nonlinear mixed effects model was fit to the projected human PK data. This human PK model was subsequently integrated with the established MetMAb exposure/anti-tumor activity relationship to simulate tumor responses at various treatment dose regimens (FIG. 9).

Monte Carlo simulations, using the human POP PK/PD model structure, parameter estimates, and variability, were conducted with Q1W and Q3W MetMAb regimens from 0-30 mg/kg/wk to project PK and tumor responses; 1000 simulations/group. The Minimum Tumorostatic Concentration (MTC), the MetMAb serum concentration yielding tumor stasis, is a measure of tumor sensitivity to drug and was derived from the modeling. MTC is calculated from the differential equation describing tumor mass where (dTM(t)/dt=0)

$$0 = K G N \cdot ( 1 - \frac{I Max \cdot C ( t )}{{IC}_{50} + C ( t )} ) \cdot T M ( t )$$

$$1 = \frac{I Max \cdot M T C}{{IC}_{50} + M T C}$$

$$M T C = \frac{{IC}_{50}}{I max - 1} = \frac{13.2 μg \text{/} mL}{1.86 - 1} = 15.3 μg \text{/} mL \\ \frac{d T M ( t )}{d t} = K G N \cdot ( 1 - \frac{I Max \cdot C ( t )}{{IC}_{50} + C ( t )} ) \cdot T M ( t )$$

Additionally, the exposure/target predictor of progression-free objective response, defined for the purposes of this experiment as ≦20% increase in tumor mass, was identified by classification and regression tree (CART) analysis (JMP 5.1 program, SAS Institute, Cary N.C.).

Results

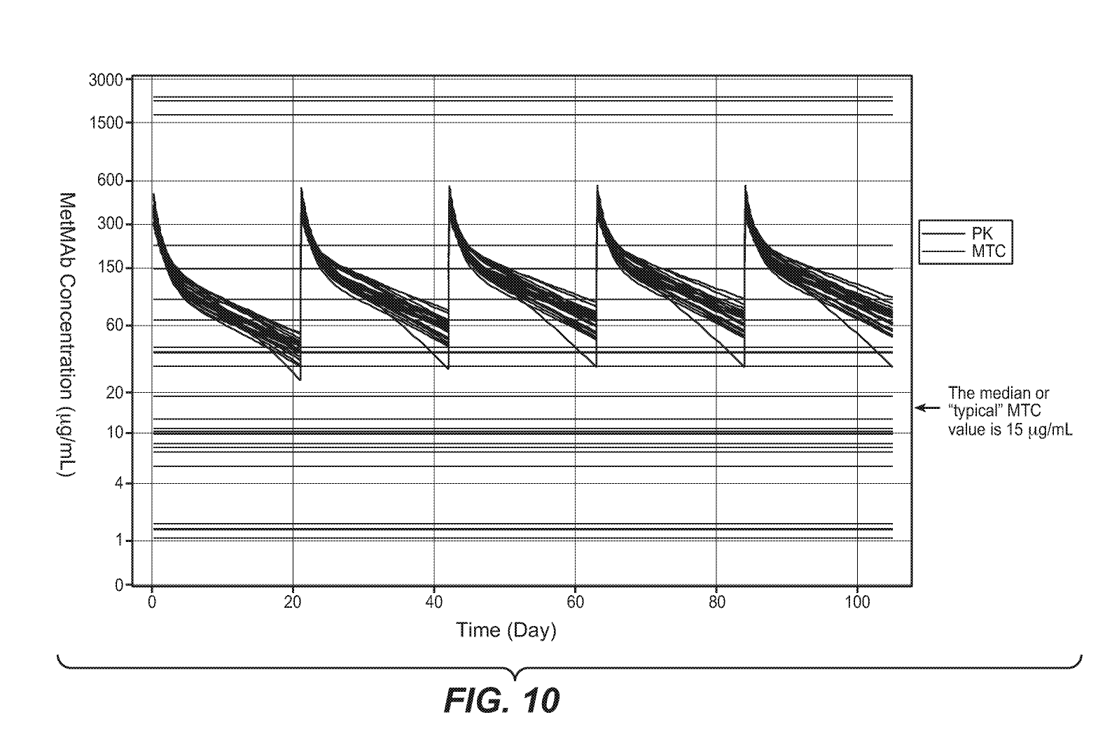

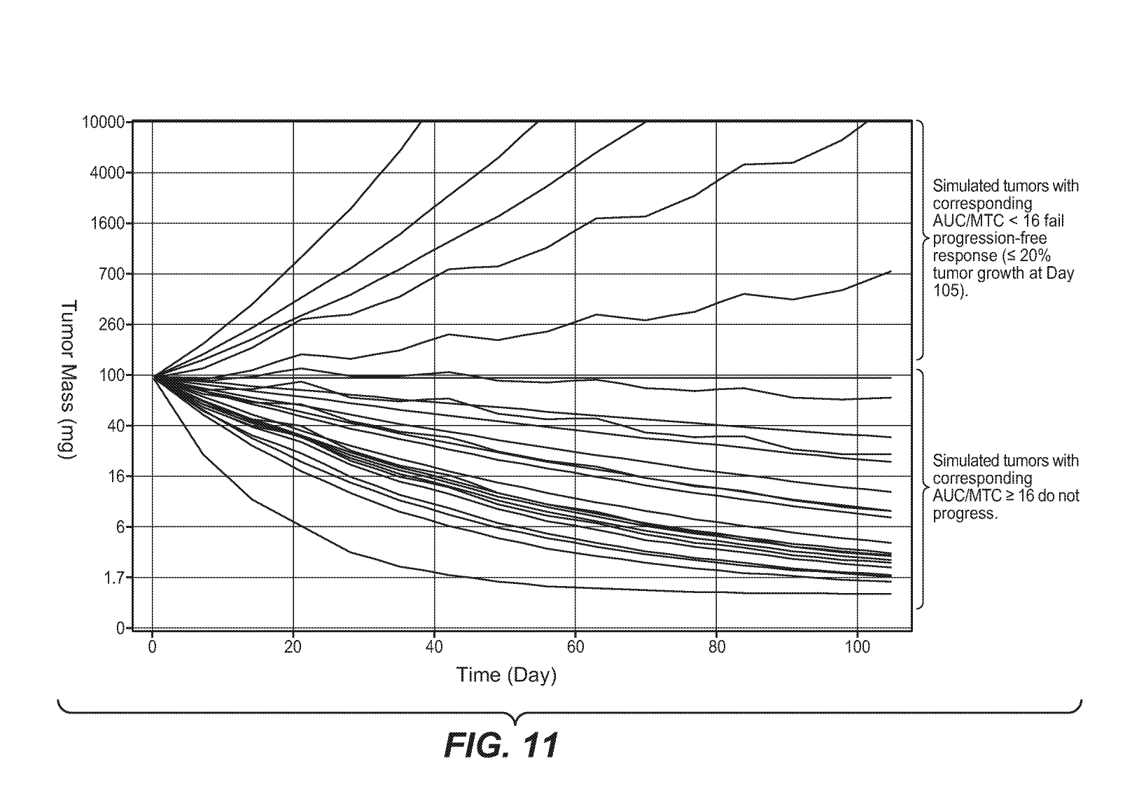

From the modeling results, individual MTC values were calculated (n=177) and the median MTC value was approximately 15 μg/mL; 90% of MTC values were below 110 μg/mL. 25 representative PK profiles and MTC values from 15 mg/kg Q3W MetMAb simulations are shown in FIG. 10. The corresponding tumor mass simulations are shown in FIG. 11.

Additionally, the exposure/target predictor of progression-free objective response, defined for the purposes of this experiment as ≦20% increase in tumor mass, was identified by classification and regression tree (CART) analysis. CART analysis identified area under the curve/tumorostatic concentration (AUC/MTC)≧16 as the breakpoint indicator of progression-free response (defined for the purposes of this experiment as ≦20% increase in tumor mass) at Day 105; simulated tumor data with MetMAb AUC/MTC≧16 are noted not to have progressed by Day 105 (see FIG. 11).

The Minimum Tumorostatic Concentration (MTC; the MetMAb serum concentration at which the tumor is neither growing nor shrinking), was estimated for MetMAb based a modeling analysis which data from preclinical mouse xenograft studies using a KP4 cell line and species-invariant time scaling to humans (Example 2). This MetMAb serum concentration was predicted to be 15 ug/mL. The pharmacokinetic data collected in the Phase I trial (Example 3) was modeled using NONMEM V (Icon Development Solutions, Ellicott City, Md. USA) in order to generate PK estimates and the variability around those estimates. These estimates and the associated variability were used to simulate 500 patient profiles in order to predict the steady-state trough concentration at various doses. FIG. 15 shows the results of this analysis. A dose of 15 mg/kg Q3W was shown to be the dose and regimen where steady-state trough concentrations were greater than the MTC in 90% of simulated patients and where an AUC/MTC greater than 16 was achieved. Based on these data, 15 mg/ml was selected to be the recommended phase II dose (see also Examples 3 and 4). The recommended Phase II dose was based on the Phase I pharmacokinetics analysis, with the goal of achieving steady-state trough concentrations greater than the MTC in 90% of patients.

In another analysis, Kaplan-Meier (KM) curves of time to progression were simulated for the MetMAb Q3W doses. For the purposes of this experiment, time to progression was defined as the time simulated tumors progress once tumors increase>20% over baseline. Similar KM curves were calculated for MetMAb Q1W doses. The comparator SOC used in this analysis had a median time to progression of 105 days and the Kaplan Meier curve for this dataset was simulated. Significant assumptions underlying this simulation experiment were the use of a simulated SOC dataset and the selection of a hazard ration≦0.75 for this experiment. From Cox proportional hazards modeling, MetMAb doses≧12.5 mg/kg Q1W and ≧20.0 mg/kg Q3W are projected to result in a significant improvement in progression-free disease (defined a priori as a hazard ratio≦0.75 for the purposes of this experiment) over comparator SOC.

### Example 3

### A Phase I Open-Label Dose-Escalation Study of the Safety and Pharmacology of MetMAb, a Monovalent Antagonist Antibody to the Receptor C-Met, Administered Intravenously in Patient with Locally Advanced or Metastatic Solid Tumors

This example describes a Phase I, open-label, dose-escalation and dose expansion study of MetMAb administered by IV infusion every 3 weeks in patients with advanced solid malignancies that are refractory to or for which there is no standard of care.

Study design. There are two stages to this study, a dose-escalation stage and an expansion stage. The dose-escalation stage was designed to evaluate the safety, tolerability, and pharmacokinetics of MetMAb delivered every 3 weeks. The design of the dose escalation stage of the study is shown in FIG. 12.

Once the recommended Phase II dose is established, additional patients are enrolled in an expansion stage to better characterize the safety, tolerability, and pharmacokinetic (PK) variability of this dose. Expansion at a dose of 15 mg/kg is performed in order to better evaluate the safety, tolerability, and PK characteristics of MetMAb in a maximum of 15 patients. The dose for the expansion phase takes into account observed toxicities, tolerability, and drug exposure. The safety, PK, and PD assessments are identical to those in the dose-escalation stage.

Approximately 27-45 patients are enrolled in this two-stage study, 21-36 in the dose-escalation stage, and 6-12 in the expansion stage. Continued dosing with MetMAb every 3 weeks (maximum of 16 cycles or 1 year) is offered to patients who derive ongoing benefit and who do not experience significant toxicity. This provides an assessment of the safety and tolerability of MetMAb with repeat dosing.

Study objectives. The primary objectives of this study were the evaluation of the safety, tolerability, and pharmacokinetics of MetMAb, when delivered every 3 weeks, to determine the MTD of MetMAb when administered every 3 weeks, and to identify a recommended Phase II dose (RP2D). The secondary objectives were the preliminarily assessment the anti-tumor activity of MetMAb, as well as the assessment of the anti-therapeutic antibody response to MetMAb. Exploratory objectives included assessment of the pharmacokinetic/pharmacodynamic and safety relationship between MetMAb serum concentration and serum levels of shed c-met and other potential serum markers that may be affected by MetMAb as well as the assessment of the expression of components of the HGF/c-met axis and/or other pathways in tumor or stromal cells (e.g., by immunohistochemistry or FISH) to assess a correlation with anti-tumor activity.

Outcome measures. The safety and tolerability of MetMAb is assessed using the following measures: frequency and nature of dose-limiting toxicities (DLTs); nature, severity, and relatedness of adverse events, graded according to the National Cancer Institute Common Terminology Criteria for Adverse Events, v3.0; changes in vital signs; and changes in clinical laboratory parameters.

The following PK parameters are derived from the serum concentration-time profile of MetMAb following administration: serum total exposure (AUC), Cₘₐₓ, clearance, volume of distribution (central compartment V_{c} and at steady state Vₛₛ), and half-life (t_{1/2β}).

The following activity outcome measures are assessed: objective response, defined as a complete or partial response confirmed≧4 weeks after initial documentation; duration of objective response; and progression-free survival. Objective response and disease progression will be determined using RECIST. ATA response to MetMAb will be derived from the frequency of ATA response and the characterization of ATA response in ATA-positive samples.

Pre- and post-dose serum is collected for evaluation of pharmacodynamic (PD) biomarkers that could be affected by inhibition of Met signaling. In addition, archival tissue is obtained for exploratory diagnostic assessments.

Patient selection criteria. Adult patients are eligible to participate in this study if they have histologic documentation of incurable, locally advanced, or metastatic solid malignancy that has failed to respond to at least one prior regimen or for which there is no standard therapy, disease that is measurable or evaluable by RECIST, life expectancy≧12 weeks, and ECOG performance status of 0-2.

Excluded subjects include subjects with primary CNS malignancy or untreated/active CNS metastases.

Study treatment. The total dose of MetMAb for each patient depended on dose level assignment and the patient's weight on, or within 14 days prior to, Day 1 of Cycle 1. Dose levels tested in Phase I were: 1 mg/kg, 4 mg/kg, 10 mg/kg, 20 mg/kg, and 30 mg/kg.

MetMAb was administered as an IV infusion. The first two doses of MetMAb for each patient were infused over 90 minutes (±10 minutes). The MetMAb infusion was slowed or interrupted for patients experiencing infusion-associated symptoms. Following the first two doses, patients were observed for 90 minutes for fever, chills, or other infusion-associated symptoms. Subsequent doses of MetMAb were administered over 30±10 minutes (for dose levels<10 mg/kg) or 60±10 minutes (for dose levels 10 mg/kg or when the final volume to be infused is 500 mL), with at least a 60-minute observation period post-infusion for all dose levels.

MetMAb. MetMAb is a known recombinant, humanized, monovalent monoclonal antibody directed against human c-met. MetMAb was provided as a lyophilized powder (400 mg) in a single-use 50-cc vial. All study drug was stored at 2 C-8 C until just before use. The solution for reconstitution was sterile water for injection and the reconstitution volume was 20.0 mL to yield a final concentration of 20 mg/mL MetMAb in 10 mM histidine succinate, 106 mM (4%) trehalose dihydrate, 0.02% polysorbate 20, pH 5.7. The total dose of MetMAb for each patient will depend on dose level assignment and the patient's weight.

Results

Twenty-one patients were enrolled in the dose-escalation phase of this study. Patient demographics are shown in Table 4.

TABLE 4
Patient Demographics

| | All Patients |
|---|---|
| Characteristic | (n = 21) |
| Age (yr) | |
| Mean (SD) | 59.9 (11.3) |
| Median | 64.0 |
| Range | 29-77 |
| Sex | |
| Female | 9 (42.9%) |
| Male | 12 (57.1%) |
| Prior therapy regimen*, n | |
| 1 | 3 |
| 2 | 5 |
| 3 | 6 |
| ≧4 | 7 |

*Includes chemotherapy, radiotherapy and targeted/biologic therapy

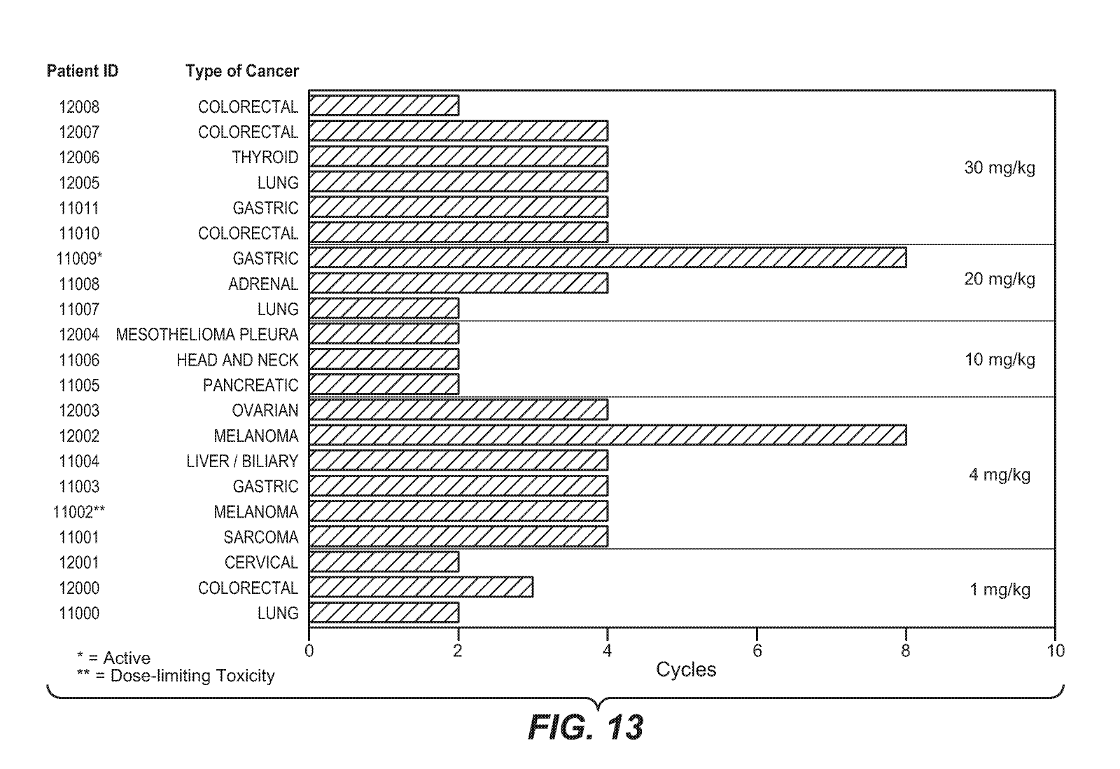

Patients received MetMAb (IV Q3W), at doses ranging from 1 mg/kg to 30 mg/kg until disease progression. A minimum of 3 patients were enrolled and observed for toxicity in each of the 5 cohorts (1, 4, 10, 20 and 30 mg/kg). The majority of the patients progressed prior to Cycle 5; one patient (melanoma) had stable disease through 8 cycles of therapy and one patient (gastric; 20 mg/kg cohort) had an objective complete response and continues to participate in the study. FIG. 13 shows patient diagnosis, treatment cohort and administered cycles for each patient in the dose-escalation stage.

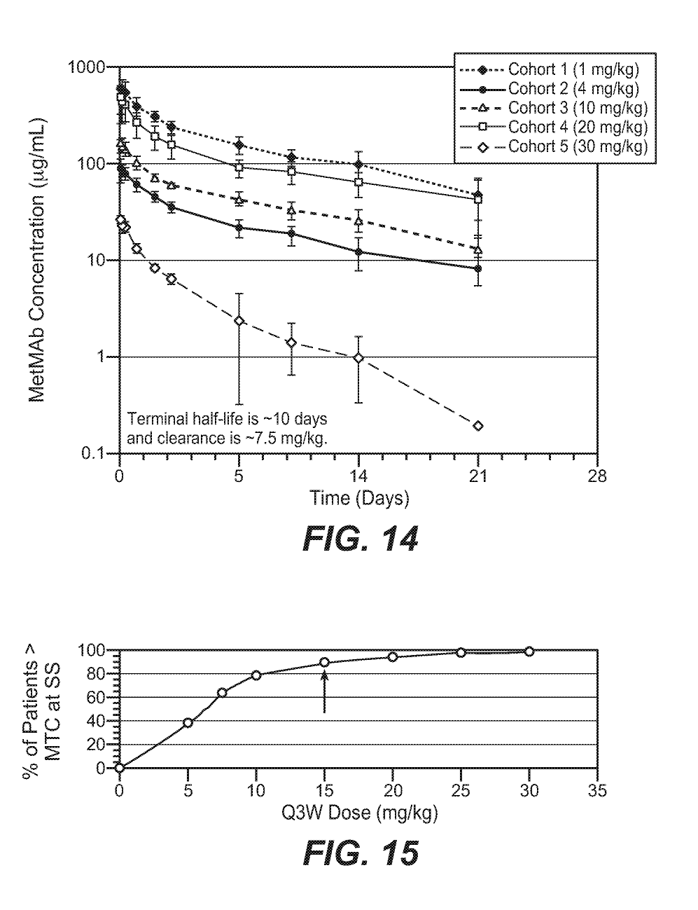

Pharmacokinetics of the study drug were determined by serially monitoring of serum samples for MetMAb throughout the study. MetMAb serum concentrations at each pharmacokinetic timepoint were averaged across all patients in each dose group. The results from the first cycle (21 days) are shown in FIG. 14.

MetMAb showed linear pharmacokinetics in the dose range 4 to 30 mg/kg. The 1 mg/kg dose had a slightly faster clearance compared to the other dose groups. Serum concentrations were similar between patients at each dose level, with an inter-individual variability less than 30%. Following MetMAb administration in the linear range, the clearance ranged from 7.4 to 9.8 mL/day/kg. The elimination rate was approximately 2.5-fold faster than standard bivalent antibodies, and was well predicted by allometric scaling of data from preclinical species. The AUC and Cₘₐₓ increased proportionally with dose, further suggesting the PK of MetMAb is linear in this dose range. The half-life of MetMAb was approximately 10 days.

The Minimum Tumorostatic Concentration (MTC; the MetMAb serum concentration at which the tumor is neither growing nor shrinking), was estimated for MetMAb based on a modeling analysis using data from preclinical mouse xenograft studies using a KP4 cell line and species-invariant time scaling to humans (Example 2). This MetMAb serum concentration was predicted to be 15 ug/mL. The pharmacokinetic data collected in the Phase I trial (Example 3) was modeled using NONMEM V (Icon Development Solutions, Ellicott City, Md. USA) in order to generate PK estimates and the variability around those estimates. These estimates and the associated variability were used to simulate 500 patient profiles in order to predict the steady-state trough concentration at various doses. FIG. 15 shows the results of this analysis. A dose of 15 mg/kg Q3W was shown to be the dose and regimen where steady-state trough concentrations were greater than the MTC in 90% of simulated patients and where an AUC/MTC greater than 16 was achieved. Based on these data, 15 mg/ml was selected to be the recommended phase II dose (see also Examples 3 and 4). The recommended Phase II dose was based on the Phase I pharmacokinetics analysis, with the goal of achieving steady-state trough concentrations greater than the MTC in 90% of patients.

A single dose limiting toxicity (DLT) of Grade 3 pyrexia occurred at 4 mg/kg; no other DLTs have been observed up to the maximum administered dose of 30 mg/kg. No drug-related Grade 4 toxicities were observed. One Grade 3 toxicity of abdominal pain was observed at 20 mg/kg. The most commonly reported adverse event was fatigue (Grade 1, 2). Table 5 shows all drug-related adverse events observed during the dose-escalation phase of the study.

MetMAb appears to be safe and generally well tolerated when administered as a single agent at doses up to 30 mg/kg, every 3 weeks. No toxicities attributed to MetMAb appear to be dose-related.

TABLE 5
All Drug-Related Adverse Events

| Total (n = 21) | | |
|---|---|---|
| | Grade 1 or 2 | Grade 3* |

| Any adverse event | 11 | (52.4%) | 2 (9.5%) |
|---|---|---|---|
| Fatigue | 7 | (33.3%) | 0 |
| Nausea | 3 | (14.3%) | 0 |
| Vomiting | 3 | (14.3%) | 0 |
| Anorexia | 2 | (9.5%) | 0 |
| Hypoalbuminaemia | 2 | (9.5%) | 0 |
| Oedema peripheral | 2 | (9.5%) | 0 |
| Abdominal pain | 0 | | 1 (4.8%) |
| Diarrhoea | 1 | (4.8%) | 0 |
| Dysgeusia | 1 | (4.8%) | 0 |
| Flushing | 1 | (4.8%) | 0 |
| Gastroesophageal reflux disease | 1 | (4.8%) | 0 |
| (GERD) | | | |
| Muscle spasms | 1 | (4.8%) | 0 |
| Mydriasis | 1 | (4.8%) | 0 |
| Oral candidiasis | 1 | (4.8%) | 0 |
| Paraesthesia oral | 1 | (4.8%) | 0 |
| Pyrexia** | 0 | | 1 (4.8%) |
| Rash | 1 | (4.8%) | 0 |
| Swelling face | 1 | (4.8%) | 0 |

*There were no Grade 4 events
**Dose Limiting Toxicity (DLT)

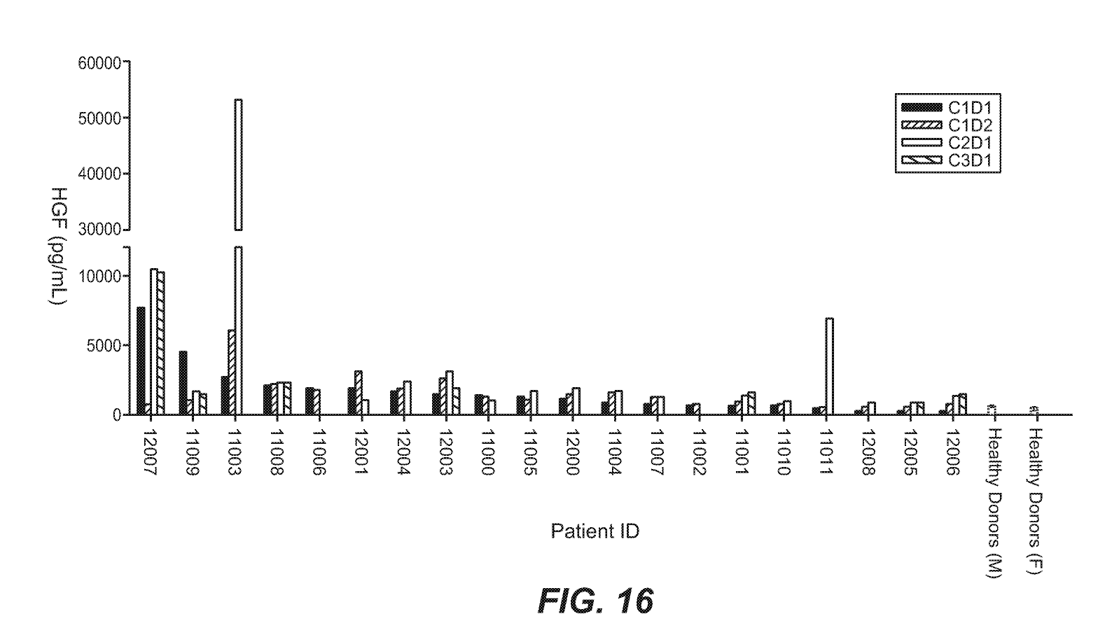

To determine whether inhibition of c-met by MetMAb treatment affected circulating HGF levels, serum HGF levels were determined for the duration of the treatment period. Serum HGF levels were determined using ELISA. FIG. 16 shows the results of this analysis. In general, there appeared to be little or no increase in HGF expression with MetMAb treatment. However, the two patients who exhibited the highest levels of baseline HGF expression showed a significant decrease in HGF expression 24 h post drug treatment. For patient 12007, HGF expression increased to baseline levels in subsequent cycles. For patient 11009, HGF levels decreased by 70% post drug treatment and remained low for the duration of the study. Circulating HGF may have utility as a biomarker of response to MetMAb therapy.

To determine whether inhibition of c-met by MetMAb treatment affected circulating IL-8 levels, serum IL-8 levels were determined for the duration of the treatment period. Serum IL-8 levels (diluted 1:5) were determined using an electrochemiluminescence based method as directed by the manufacturer (Meso Scale Discovery, Gaithersburg Md.; Cat. No. K111ANC).

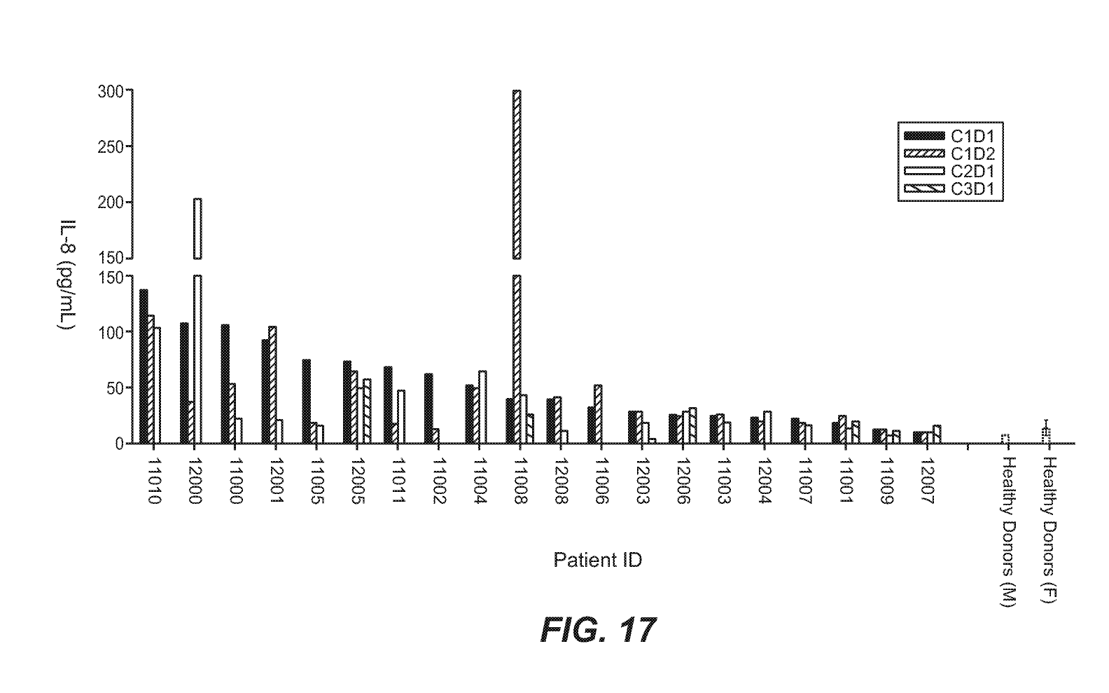

The results of this experiment are shown in FIG. 17. Baseline IL-8 expression in the study group varied significantly from 4-107 pg/ml. Following treatment (24 h), subjects with high physiologic levels of IL-8 (>50 pg/ml) showed a greater than 50% reduction in circulating IL-8. In subjects with less than 50 pg/ml baseline IL-8, expression post-MetMAb treatment did not change significantly. Circulating IL-8 level may have utility as a marker of response to MetMAb treatment.

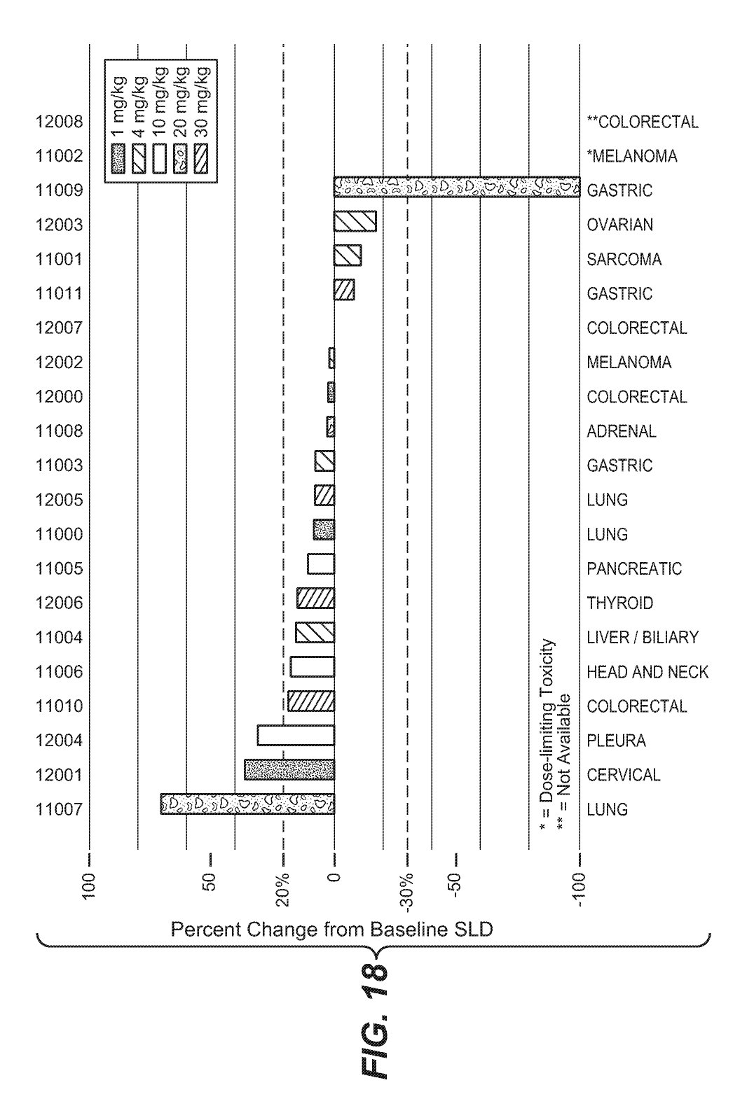

FIG. 18 shows the best tumor response of all the patients who participated in the dose escalation stage. One patient was not assessed as the patient progressed before the first evaluation timepoint; another patient's CT evaluation was not available at the time these data were collected. A complete, objective response was seen in one gastric cancer patient in the 20 mg/kg cohort. A best response of stable disease was seen in 15 out of the 21 patients. Three patients had progressive disease.

Patient 11009 is a 50 year-old female gastric adenocarcinoma patient with metastatic liver lesion as site of measurable disease. This patient was diagnosed in April 2007 (T1N1M1, serosal implant on the gallbladder) and received FOLFOX6 from May 29, 2007 through Aug., 13 2007. The patient's disease progressed in Aug. 22, 2007 and she was then treated with an investigative therapy from Oct. 18, 2007 through to Jan. 31, 2007. The patient's disease progressed again and she was enrolled in the MetMAb phase I study in March 2008 with a 7×11 mm lesion on a spiral CT. While on this trial the patient had stable disease on her first evaluation (Apr. 29, 2008) and a complete response on Jun. 13, 2008. This CT response was confirmed with another CT (July 2008). MRI imaging showed no evidence of disease in September 2008. This patient's tumor sample showed intracellular staining of HGF (by IHC analysis), suggesting that the patient's tumor possessed autocrine biology.

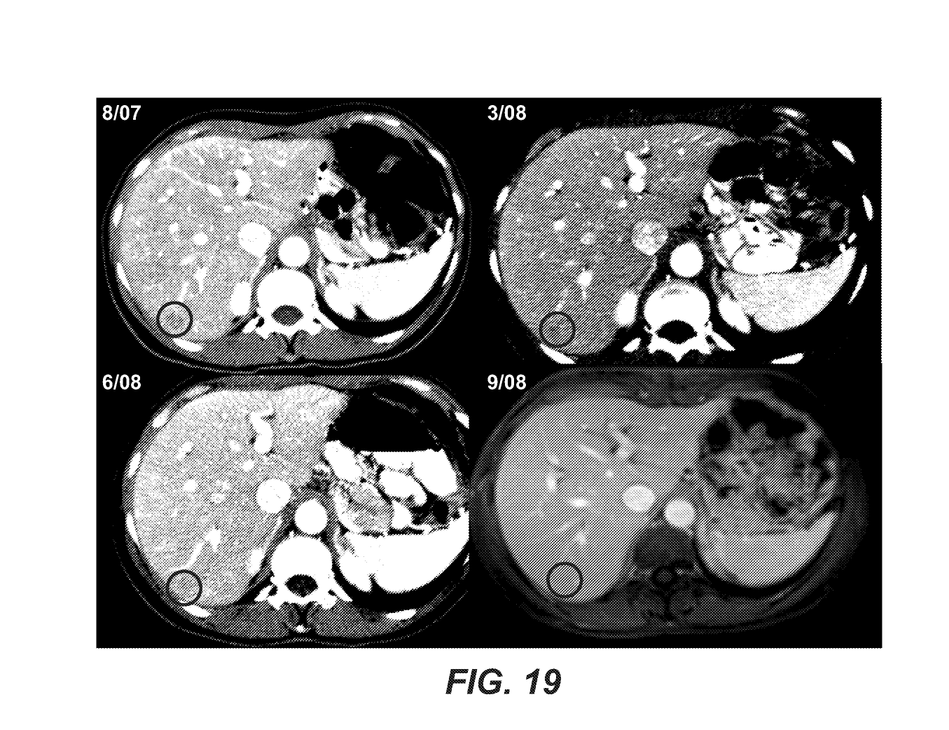

FIG. 19 shows the CT and MRI scans of patient 11009 prior to and after MetMAb treatment. Upper panels (L and R) are prior to MetMAb treatment. Lower panels (L and R) are CT and MRI scans that confirmed complete response. Disappearance of all target lesions was confirmed after more than 4 weeks.

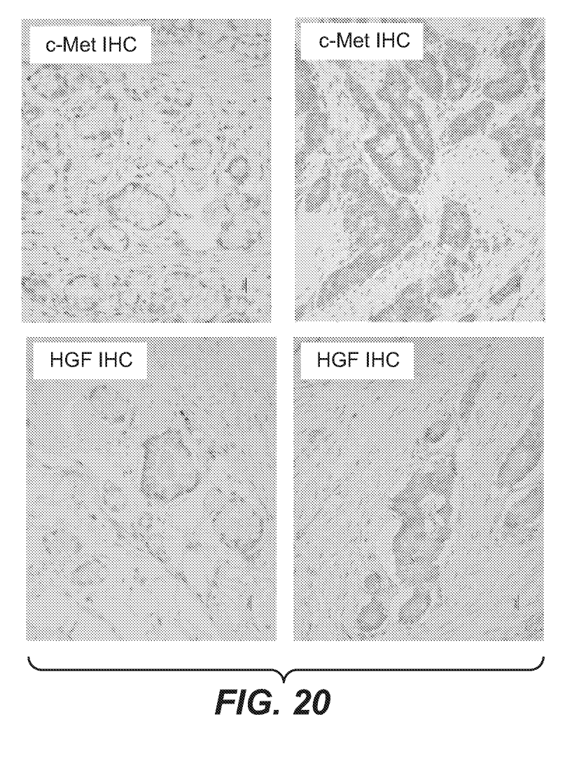

FIG. 20 shows immunohistochemical staining of archival tumor tissue from patient 11009. Immunohistochemical analysis to detect c-met protein was performed, revealing moderate membraneous and cytoplasmic c-Met expression and cytoplasmic and peri-membraneous HGF expression in tumor cells present in the tumor sample.

FISH analysis was performed on an archival tumor sample from patient 11009. FISH analysis revealed a high polysomy of the c-met gene as compared to chromosomal 7 control.

### Example 4

### A Phase II Study to Determine the Safety and Activity of MetMAb, a Monovalent Antagonist Antibody to the Receptor C-Met, Administered Intravenously, in Patients with Non-Small Cell Lung Cancer, in Combination with TARCEVA® (Erlotinib) (OAM4558g)

Lung cancer remains one of the leading causes of cancer death worldwide; it is the second most common cancer in both men and women, and accounts for approximately 15% of all new cancers. In 2008, it is estimated that there will be approximately 215,000 new cases of lung cancer and an estimated 160,000 deaths. Only about 15% of people diagnosed with lung cancer stay alive after 5 years. NSCLC is one of the two major types of lung cancer, accounting for approximately 85% of all lung cancer cases.

This example provides a method of treating NSCLC with a combination of anti-c-met antibody and an EGFR inhibitor, which can result in meaningful clinical benefit, by administering to a subject an effective dose of anti-c-met antagonist antibody and an EGFR inhibitor. For example, in certain embodiments, a subject is administered: (1) MetMAb at 15 mg/kg (e.g., based on subject's weight at Day 1) at day one of a 21 day cycle; and (2) erlotinib, typically administered orally, at a dose of 150 mg, each day of a 21 day cycle.

In pre-clinical animal models, treatment with the combination of MetMAb and erlotinib resulted in highly significant improvements in tumor growth inhibition and tumor progression relative to treatment with MetMAb or erlotinib alone. See co-owned, co-pending US patent publication no. 2009/0226443.

Protocol synopsis. A blind, Phase II, randomized, multicenter trial designed to evaluate preliminary activity and safety of treatment with MetMAb plus erlotinib versus erlotinib plus placebo in NSCLC.

Objectives. The primary objective of this study is to evaluate progression-free survival (PFS) of MetMAb plus Erlotinib, relative to Erlotinib plus placebo, in patients with Met positive tumors (as determined by immunohistochemistry), as well as all patients (i.e., including patients with Met negative tumors).

The secondary objectives of this study are: (a) to determine the overall RECIST response rate and duration of response in patients with c-met positive tumors, as well as overall; (b) to characterize the safety and tolerability of MetMAb plus Erlotinib in patients with NSCLC; and (c) to evaluate minimum concentration (Cmin) and maximum concentration (Cmax) of both MetMAb and erlotinib in patients with NSCLC.

Additional objectives of this study are to (a) to evaluate overall survival, in patients with c-met positive tumors as well as overall; (b) to evaluate the FDG-PET response rate by treatment group and in patients with c-met positive tumors, as well as overall; (c) to evaluate progression-free survival (PFS) in FDG-PET responders versus non-responders, by treatment group and in Met positive tumors, as well as overall; (d) to evaluate the relationship between Response Evaluation Criteria In Solid Tumors (RECIST response at first tumor assessment and PFS; (e) to evaluate the relationship between response and changes in biomarkers (or baseline expression of) related to the HGF/Met and/or EGFR signaling pathways (including, but not limited to IL8 and serum HGF); (f) to evaluate potential mechanisms of resistance in patients who progress on study; and (g) evaluate time to progression in patients with c-met positive tumors as well as overall.

Study design. This study is a Phase II, double-blind, randomized, multicenter trial designed to evaluate the preliminary activity and safety of treatment with MetMAb plus Erlotinib versus Erlotinib plus placebo in second and third-line NSCLC. Approximately 120 patients from approximately 60 sites will be randomized in a 1:1 ratio to one of the two treatment arms: MetMAb plus Erlotinib vs. Erlotinib plus placebo. Randomization is stratified by smoking status (non-smokers and smokers who have quit more than 10 years ago versus current smokers and smokers who have quit less than 10 years ago), performance status and histology (squamous, non-squamous, not otherwise specified). Treatment in each arm is continued until progression of disease, unacceptable toxicity, or any other discontinuation criterion is met. Upon disease progression, patients randomized to the Erlotinib plus placebo arm are given the option to receive MetMAb (in addition to continuing Erlotinib), provided they continue to meet eligibility criteria. Safety data collected from this cross-over is summarized for hypothesis generating purposes.

During the study, data on tumor measurement and survival status are collected for evaluation of PFS, overall survival (OS) and overall response rate (ORR). CT scans are obtained at baseline and for the first four cycles at an approximately every 6 week intervals (i.e., every two three-week cycles of MetMAb/placebo). After four cycles, routine CT scans are performed approximately every 9 weeks (every 3 cycles of MetMAb/placebo). FDG-PET imaging is obtained at baseline and at Day 10-14 of Cycle 1. After 60 patients are randomized and have had their 12 week follow-up, an interim analysis is performed to determine activity overall. Based on the results of this interim analysis, the study may be modified to enrich for a specific NSCLC subtype or some assessments may be discontinued.

In some patients, exploratory serum and plasma samples are collected to determine the effect of MetMAb plus Erlotinib on circulating levels of potential markers of activity, including but not limited to IL-8 and HGF. Correlating these and other markers with clinical outcomes assists in identifying predictive biomarkers, e.g., markers in circulation that may reflect drug activity or response to therapy. Blood for serum and plasma is drawn from consenting patients at pre-specified times and evaluated for levels of these exploratory markers.

Expression of c-met and/or EGFR is determined in a pre-treatment sample of the tumor. C-met and/or EGFR expression is determined by IHC and/or FISH analysis.

Because of the well-established survival benefit of Eastern Asians when treated with EGFR-directed therapies, this study will not allow more than 20% of the evaluable study population to be Eastern Asians.

Outcome measures. The primary outcome measure of this study is progression free survival (PFS) defined by the Response Evaluation Criteria In Solid Tumors (RECIST)) or death from any cause within thirty days of the last treatment.

The secondary outcome measures for this study are as follows:

(a) overall response (OR) (partial response plus complete response) as determined using RECIST in Met positive tumors and overall; and

(b) duration of OR.

Exploratory outcome measures include the following:

(a) FDG-PET response rates, as determined based on the definitions of the European Organization for Research of Cancer (EORTC);

(b) Incidence, nature and severity of adverse events and serious adverse events, and changes in vital signs, physical findings, and clinical laboratory results during and following study drug administration will be monitored; and

(c) Overall survival (time from randomization until death from any cause in patients with c-met positive tumors and overall).

Serum samples will be collected for analysis of MetMAb and erlotinib pharmacokinetics and pharmacodynamics.

Patient selection criteria. Adult patients are eligible to participate in this study if they have inoperable locally advanced or metastatic (Stage IIIb/IV) NSCLC (e.g., as determined by histological studies) and have received at least one, but no more than two prior regimens for Stage IIIb/IV NSCLC disease. In this study, cancer staging will follow the American Joint Committee on Cancer's AJCC Cancer Staging Manual. Patients who receive neo-adjuvant and/or adjuvant therapy for Stage I-IIIa disease prior to their first-line regiment (for Stage IIIb/IV) are eligible for study participation, provided they also receive first-line therapy for Stage IIIb/IV disease. In some embodiments, at least one of the chemotherapy containing regimens (for any stage) must have been platinum-based. Patients must have measurable disease as determined by RECIST. In some embodiments, patients must have at least one measurable lesion on a pre-treatment FDG-PET scan that is also a target lesion on CT according to RECIST. In some embodiments, patients must provide a pre-treatment tumor specimen, and possess at least one measurable lesion on a pre-treatment FDG-PET scan that is also a target lesion on CT according to RESIST.

In some embodiments, excluded subjects are subjects who have had more than two prior treatments for Stage IIIB/IV. In some embodiments, excluded subjects include subjects with more than 30 days of exposure to an investigational or marketed agent that can act by EGFR inhibition, or a known EGFR-related toxicity resulting in dose modifications. EGFR inhibitors include (but are not limited to) gefitinib, erlotinib, and cetuximab. In some embodiments, excluded subjects include subjects who have received chemotherapy, biologic therapy, radiotherapy or investigational drug within 28 days prior to randomization (except that kinase inhibitors may be used within two weeks prior to randomization provided any drug related toxicity has adequately resolved), subjects, or subjects with untreated and/or active (progressing or requiring anticonvulsants or corticosteroids for symptomatic control) CNS metastasis. In some embodiments, subjects with history of brain metastasis may be eligible for study participation, as long as they meet the following criteria: (a) measurable disease outside the CNS, as defined by RECIST; (b) no radiographic evidence of interim progression between the completion of CNS-directed therapy and the screening radiographic study; (c) CNS-directed treatment which may include neurosurgery or stereotactic radiosurgery; (d) the screening of CNS radiographic study is ≧4 weeks since completion of radiotherapy and ≧2 weeks since the discontinuation of corticosteroids and anticonvulsants; (e) radiotherapy and stereotactic radiosurgery must be completed 4 weeks prior to Day 1; and (f) neurosurgery must be completed≧24 weeks prior to Day 1, and brain biopsy must be completed≧12 weeks prior to Day 1.

In some embodiment, excluded subject also includes subjects with history of serious systemic disease, including myocardial infarction within the last 6 months prior to randomization, uncontrolled hypertension (blood pressure>150/100 mmHg on medication), unstable angina, New York Heart Association (NYHA) Grade II or greater congestive heart failure, unstable symptomatic arrhythmia requiring medication (patients with chronic atrial arrhythmia, i.e., atrial fibrillation or paroxysmal supraventricular tachycardia are eligible), or Grade II or greater peripheral vascular disease; uncontrolled diabetes as evidenced by fasting serum glucose level>200 mg/dL; major surgical procedure or significant traumatic injury within 28 days prior to randomization; anticipation of need for a major surgical procedure during the course of the study; local palliative radiotherapy within 7 or 14 days prior to randomization or persistent adverse effects from radiotherapy that have not been resolved to Grade II or less prior to randomization; inability to take oral medication or requirement for IV alimentation or total parenteral nutrition with lipids, or prior surgical procedures affecting gastrointestinal absorption. In some embodiments, excluded subjects include subjects having any of the following abnormal hematologic values (within 2 weeks prior to randomization): ANC<1,500 cells/mL, Platelet count<100,000 cells/mL, Hemoglobin<9.0 g/dL, following RBC transfusion, Other baseline laboratory values (within 2 weeks prior to randomization), Serum bilirubin>1.5×ULN, Serum creatinine>1.5×ULN, Uncontrolled hypercalcemia (>11.5 mg/dL or >1.5 ionized calcium). In some embodiments, excluded subject include subjects having uncontrolled diabetes and subjects having symptomatic hypercalcemia requiring continued use of bisphosphonate therapy.

In some embodiments, excluded subjects include pregnant or breast-feeding women; subjects having other malignancies that have undergone a putative surgical cure (i.e., intraepithelial carcinoma of the cervix uteri, localized prostate cancer post prostatectomy, or basal/squamous cell carcinoma of the skin) within 5 years prior to randomization may be discussed with the medical monitor; or evidence of confusion or disorientation, or history of major psychiatric illness. See also additional exclusions on the label of erlotinib.

Trial drugs. MetMAb is a known recombinant, humanized, monovalent monoclonal antibody directed against c-met. MetMAb is supplied as a sterile liquid in a single-use 15-cc vial. Each vial contains 600 mg of MetMAb in 10 ml at a concentration of 60 mg/ml in 10 mM histidine acetate, 120 nM trehalose, 0.02% polysorbate 20, pH 5.4. MetMAb vials are refrigerated at 2 C-8 C and remain refrigerated until just prior to use. MetMAb is administered intravenously, after dilution in normal saline (0.9%).

Erlotinib (TARCEVA®) is provided as a conventional, immediate-release tablet containing erlotinib as the hydrochloride salt. In addition to the active ingredient, erlotinib, tablets contain lactose (hydrous), microcrystalline cellulose, sodium starch glycolate, sodium lauryl sulfate and magnesium stearate. Tablets containing 25 mg, 100 mg and 150 mg of Erlotinib are available.

Placebo will consist of 250 cc 0.9% NSS (Saline IV solution, 0.9%).

Study treatment. The dose of MetMAb will be 15 mg/kg intravenously on Day 1 of a 3-week cycle. The weight at screening will be used to determine the actual dose of MetMAb. The dose of erlotinib will be 150 mg by mouth each day of a 3-week cycle. Dosage level for erlotinib may be reduced to 100 mg (first reduction) or 50 mg (second reduction) for toxicity likely attributable to erlotinib (e.g., rash, diarrhea).

Results

Administration of (1) MetMAb at 15 mg/kg (e.g., based on subject's weight at Day 1 or at screening) at day one of a 21 day cycle; and (2) erlotinib, typically administered orally, at a dose of 150 mg, each day of a 21 day cycle, to subjects with non-small cell carcinoma extended time to disease progression (TTP) and/or progression-free survival, and survival.

### Example 5

### Treatment of Glioblastoma Using C-Met Antagonist Antibody

This example provides a method of treating glioblastoma with an anti-c-met antibody, which can result in clinically meaningful benefit, by administering to a subject an effective dose of anti-c-met antagonist antibody. For example, in certain embodiments, a subject is administered: MetMAb at 15 mg/kg (e.g., based on subject's weight at Day 1) at day one of a 21 day cycle. In certain embodiments, MetMAb is administered in combination with the standard of care and/or other approved therapies.

### Example 6

### Treatment of Pancreatic Cancer Using C-Met Antagonist Antibody

This example provides a method of treating pancreatic cancer with an anti-c-met antibody, which can result in clinically meaningful benefit, by administering to a subject an effective dose of anti-c-met antagonist antibody. For example, in certain embodiments, a subject is administered: MetMAb at 15 mg/kg (e.g., based on subject's weight at Day 1) at day one of a 21 day cycle. In certain embodiments, MetMAb is administered in combination with the standard of care and/or other approved therapies.

### Example 7

### Treatment of Sarcoma Using C-Met Antagonist Antibody

This example provides a method of treating myosarcoma with an anti-c-met antibody, which can result in clinically meaningful benefit, by administering to a subject an effective dose of anti-c-met antagonist antibody. For example, in certain embodiments, a subject is administered: MetMAb at 15 mg/kg (e.g., based on subject's weight at Day 1) at day one of a 21 day cycle. In certain embodiments, MetMAb is administered in combination with the standard of care and/or other approved therapies.

### Example 8

### Treatment of Renal Cell Carcinoma Using C-Met Antagonist Antibody

This example provides a method of treating renal cell carcinoma with an anti-c-met antibody, which can result in clinically meaningful benefit, by administering to a subject an effective dose of anti-c-met antagonist antibody. For example, in certain embodiments, a subject is administered: MetMAb at 15 mg/kg (e.g., based on subject's weight at Day 1) at day one of a 21 day cycle. In certain embodiments, MetMAb is administered in combination with the standard of care and/or other approved therapies.

### Example 9

### Treatment of Gastric Carcinoma Using C-Met Antagonist Antibody

This example provides a method of treating gastric carcinoma with an anti-c-met antibody, which can result in clinically meaningful benefit, by administering to a subject an effective dose of anti-c-met antagonist antibody. For example, in certain embodiments, a subject is administered: MetMAb at 15 mg/kg (e.g., based on subject's weight at Day 1) at day one of a 21 day cycle. In certain embodiments, MetMAb is administered in combination with the standard of care and/or other approved therapies.

### Example 10

### Treatment of Colorectal Cancer Using C-Met Antagonist Antibody

This example provides a method of treating colorectal cancer with an anti-c-met antibody, which can result in clinically meaningful benefit, by administering to a subject an effective dose of anti-c-met antagonist antibody. For example, in certain embodiments, a subject is administered: MetMAb at 15 mg/kg (e.g., based on subject's weight at Day 1) at day one of a 21 day cycle. In certain embodiments, MetMAb is administered in combination with the standard of care and/or other approved therapies.

### Example 11

### Treatment of Breast Cancer Using C-Met Antagonist Antibody

This example provides a method of treating breast cancer with an anti-c-met antibody, which can result in clinically meaningful benefit, by administering to a subject an effective dose of anti-c-met antagonist antibody. For example, in certain embodiments, a subject is administered: MetMAb at 15 mg/kg (e.g., based on subject's weight at Day 1) at day one of a 21 day cycle. In certain embodiments, MetMAb is administered in combination with the standard of care and/or other approved therapies.

Although the foregoing invention has been described in some detail by way of illustration and example for purposes of clarity of understanding, the descriptions and examples should not be construed as limiting the scope of the invention.

1. A method of treating cancer in a subject, comprising administering to the subject an anti-c-met antibody at a dose of about 15 mg/kg every three weeks.

2. A method of treating cancer in a subject, comprising administering to the subject (a) an anti-c-met antibody at a dose of about 15 mg/kg every three weeks; and (b) an EGFR antagonist.

3. The method of claim 1 or 2, wherein the antibody comprises a single antigen binding arm and comprises a Fc region, wherein the Fc region comprises a first and a second Fc polypeptide, wherein the first and second Fc polypeptides are present in a complex and form a Fc region that increases stability of said antibody fragment compared to a Fab molecule comprising said antigen binding arm.

4. The method of claim 1 or 2, wherein the antibody comprises (a) a first polypeptide comprising a heavy chain variable domain having the sequence:
EVQLVESGGGLVQPGGSLRLSCAASGYTFTSYWLHWVRQAPGKGLEWVGMIDPS NSDTRFNPNFKDRFTISADTSKNTAYLQMNSLRAEDTAVYYCATYRSYVTPLDYWGQGT LVTVSS (SEQ ID NO:10), CH1 sequence, and a first Fc polypeptide; (b) a second polypeptide comprising a light chain variable domain having the sequence:
DIQMTQSPSSLSASVGDRVTITCKSSQSLLYTSSQKNYLAWYQQKPGKAPKLLIYWASTR ESGVPSRFSGSGSGTDFTLTISSLQPEDFATYYCQQYYAYPWTFGQGTKVEIKR (SEQ ID NO:11), and CL1 sequence; and (c) a third polypeptide comprising a second Fc polypeptide, wherein the heavy chain variable domain and the light chain variable domain are present as a complex and form a single antigen binding arm, wherein the first and second Fc polypeptides are present in a complex and form a Fc region that increases stability of said antibody fragment compared to a Fab molecule comprising said antigen binding arm.

5. The method of claim 4, wherein the first polypeptide comprises the Fc sequence depicted in FIG. 1 (SEQ ID NO: 12) and the second polypeptide comprises the Fc sequence depicted in FIG. 2 (SEQ ID NO: 13).

6. The method of claim 4, wherein the first polypeptide comprises the Fc sequence depicted in FIG. 2 (SEQ ID NO: 13) and the second polypeptide comprises the Fc sequence depicted in FIG. 1 (SEQ ID NO: 12).

7. The method of claim 4, wherein the antibody is MetMAb.

8-9. (canceled)

10. The method of claim 7, wherein the EGFR antagonist is N-(3-ethynylphenyl)-6,7-bis(2-methoxyethoxy)-4-quinazolinamine.

11-15. (canceled)

16. The method of claim 10, wherein the cancer is selected from the group consisting of non-small cell lung cancer, renal cell cancer, pancreatic cancer, gastric carcinoma, bladder cancer, esophageal cancer, mesothelioma, melanoma, breast cancer, thyroid cancer, colorectal cancer, head and neck cancer, osteosarcoma, prostate cancer, or glioblastoma.

17. The method of claim 16, wherein the cancer is non-small cell lung cancer.

18. The method of claim 1 or 2, wherein the anti-cmet antibody is MetMAb, the EGFR antagonist is N-(3-ethynylphenyl)-6,7-bis(2-methoxyethoxy)-4-quinazolinamine and the cancer is non-small cell lung cancer, wherein the EGFR antagonist is administered at a dose of 150 mg, each day of a three week cycle.

19. The method of claim 18, further comprising administering a third therapeutic agent to the subject.

20. The method of claim 19, wherein the third therapeutic agent is selected from the group consisting of chemotherapeutic agent, VEGF antagonist, antimetabolite compound, antibody directed against a tumor associated antigen, anti-hormonal compound, cardioprotectant, cytokine, anti-angiogenic agent, tyrosine kinase inhibitor, COX inhibitor, non-steroidal anti-inflammatory drug, farnesyl transferase inhibitor, antibody that binds oncofetal protein CA 125, Raf or ras inhibitor, liposomal doxorubicin, topotecan, taxane, dual tyrosine kinase inhibitor, TLK286, EMD-7200, a medicament that treats nausea, a medicament that prevents or treats skin rash or standard acne therapy, a medicament that treats or prevents diarrhea, a body temperature-reducing medicament, and a hematopoietic growth factor.

21. The method of claim 20, wherein the third therapeutic agent is a VEGF antagonist.

22. The method of claim 21, wherein the VEGF antagonist is bevacizumab.

23. (canceled)

24. The method of claim 18, wherein serum from the subject displays high levels of IL8 expression.

25. A method for evaluation of a patient undergoing treatment for cancer, the method comprising: predicting cancer prognosis of the patient based on a comparison of expression of IL8 in a biological sample from the patient with expression of IL8 in the patient biological sample taken prior to treatment, wherein decreased IL8 expression in the serum of the patient undergoing treatment relative to expression in the pre-treatment sample is prognostic for cancer in the patient.

26. A method for evaluation of a patient having or suspected of having cancer, the method comprising: predicting cancer prognosis of the patient based on a comparison of expression of IL8 in a biological sample from the patient with expression of IL8 in a control sample; wherein IL8 expression in the patient biological sample relative to the control sample is prognostic for cancer in the patient.
